(19) United States
(12) Patent Application Publication
(10) Pub. No.: US 2013/0023033 A1
(43) Pub. Date: Jan. 24, 2013

(54) PHARMACOLOGICALLY INDUCED TRANSGENE ABLATION SYSTEM

(71) Applicants: James M. Wilson, Glen Mills, PA (US); Shu-Jen Chen, North Wales, PA (US); Anna P. Tretiakova, Philadelphia, PA (US)

(72) Inventors: James M. Wilson, Glen Mills, PA (US); Shu-Jen Chen, North Wales, PA (US); Anna P. Tretiakova, Philadelphia, PA (US)

(73) Assignee: The Trustees of the University of Pennsylvania, Philadelphia, PA (US)

(21) Appl. No.: 13/638,015

(22) PCT Filed: Mar. 28, 2011
(86) PCT No.: PCT/US2011/030213

§ 371 Date: Sep. 28, 2012

Related U.S. Application Data
(60) Provisional application No. 61/318,752, filed on Mar. 29, 2010.

(51) Int. Cl. C12N 7/01; C12N 5/10; C12N 1/21; C12N 15/63

(52) U.S. Cl. 435/235.1; 435/320.1; 435/419; 435/252.3; 435/325

(57) ABSTRACT

The present invention relates to gene therapy systems designed for the delivery of a therapeutic product to a subject using replication-defective virus composition(s) engineered with a built-in safety mechanism for ablating the therapeutic gene product, either permanently or temporarily, in response to a pharmacological agent—preferably an oral formulation, e.g., a pill. The invention is based, in part, on the applicants' development of an integrated approach, referred to herein as “PITA” (Pharmacologically Induced Transgene Ablation), for ablating a transgene or negatively regulating transgene expression. In this approach, replication-deficient viruses are used to deliver a transgene encoding a therapeutic product (an RNA or a protein) so that it is expressed in the subject, but can be reversibly or irreversibly turned off by administering the pharmacological agent; e.g., by administration of a small molecule that induces expression of an ablator specific for the transgene or its RNA transcript.

### 1. INTRODUCTION

The present invention relates to gene therapy systems designed for the delivery of a therapeutic product to a subject using replication-defective virus composition(s) engineered with a built-in safety mechanism for ablating the therapeutic gene product, either permanently or temporarily, in response to a pharmacological agent—preferably an oral formulation, e.g., a pill.

### 2. BACKGROUND OF THE INVENTION

Gene therapy involves the introduction of genetic material into host cells with the goal of treating or curing disease. Many diseases are caused by “defective” genes that result in a deficiency in an essential protein. One approach for correcting faulty gene expression is to insert a normal gene (transgene)) into a nonspecific location within the genome to replace a nonfunctional, or “defective,” disease-causing gene. Gene therapy can also be used as a platform for the delivery of a therapeutic protein or RNA to treat various diseases so that the therapeutic product is expressed for a prolonged period of time, eliminating the need for repeat dosing. A carrier molecule called a vector must be used to deliver a transgene to the patient's target cells, the most common vector being a virus that has been genetically altered to carry normal human genes. Viruses have evolved a way of encapsulating and delivering their genes to human cells in a pathogenic manner and thus, virus genomes can be manipulated to insert therapeutic genes.

Stable transgene expression can be achieved following in vivo delivery of vectors based on adenoviruses or adeno-associated viruses (AAVs) into non dividing cells, and also by transplantation of stem cells transduced ex vivo with integrating and non-integrating vectors, such as those based on retroviruses and lentiviruses. AAV vectors are used for gene therapy because, among other reasons, AAV is nonpathogenic, it does not elicit a deleterious immune response, and AAV transgene expression frequently persists for years or the lifetime of the animal model (see Shyam et al., Clin. Microbiol. Rev. 24(4):583-593). AAV is a small, nonenveloped human parvovirus that packages a linear strand of single stranded DNA genome that is 4.7 kb. Productive infection by AAV occurs only in the presence of a helper virus, either adenovirus or herpes virus. In the absence of a helper virus, AAV integrates into a specific point of the host genome (19q 13-qter) at a high frequency, making AAV the only mammalian DNA virus known to be capable of site-specific integration. See, Kotin et at., 1990, PNAS, 87: 2211-2215. However, recombinant AAV, which does not contain any viral genes and only a therapeutic gene, does not integrate into the genome. Instead the recombinant viral genome fuses at its ends via inverted terminal repeats to form circular, episomal farms which are predicted to be the primary cause of the long term gene expression (see Shyam et at., Clin. Microbiol. Rev. 24(4):583-593).

Virtually all pre-clinical and clinical applications of gene therapy have used vectors that express the transgene from a constitutive promoter, which means it is active at a fixed level for as long as the vector genome persists. However, many diseases that are amenable to gene therapy may need to have expression of the transgene regulated. Several systems have been described which that are based on the general principle of placing a gene of interest under the control of a drug-inducible engineered transcription factor in order to positively induce gene expression (Mason et at., 1997, Curr Opin Chem Biol, 1 (2): 210-8; Rossi et at., Curr Opin Biotechnol, 1998. 9(5): p. 451-6). The various systems can be divided into two classes. In the first, a DNA-binding domain that is allosterically regulated by inducers such as tetracyclines, antiprogestins, or ecdysteroids is coupled to a transactivation domain. The addition (or in some cases removal) of the drug leads to DNA binding and hence transcriptional activation. In the second, allosteric control is replaced with the more general mechanism of induced proximity. DNA binding and activation domains are expressed as separate polypeptides that are reconstituted into an active transcription factor by addition of a bivalent small molecule, referred to as a chemical inducer of dimerization or “dimerizer.” While these systems are useful in gene therapy systems that require inducing transgene expression, they have not addressed the need to be able to turn off or permanently ablate transgene expression if it is no longer needed or if toxicity due to long-term drug administration ensues.

### 3. SUMMARY OF THE INVENTION

The present invention relates to gene therapy systems designed for the delivery of a therapeutic product to a subject using replication-defective virus composition(s) engineered with a built-in safety mechanism for ablating the therapeutic gene product, either permanently or temporarily, in response to a pharmacological agent—preferably an oral formulation, e.g., a pill.

The invention is based, in part, on the applicants' development of an integrated approach, referred to herein as “PITA” (Pharmacologically Induced Transgene Ablation), for ablating a transgene or negatively regulating transgene expression. In this approach, replication-deficient viruses are used to deliver a transgene encoding a therapeutic product (an RNA or a protein) so that it is expressed in the subject, but can be reversibly or irreversibly turned off by administering the pharmacological agent.

The invention presents many advantages over systems in which expression of the transgene is positively regulated by a pharmacological agent. In such cases, the recipient must take a pharmaceutic for the duration of the time he/she needs the transgene expressed—a duration that may be very long and may be associated with its own toxicity.

In one aspect, the invention provides a replication-defective virus composition suitable for use in human subjects in which the viral genome has been engineered to contain: (a) a first transcription unit that encodes a therapeutic product in operative association with a promoter that controls transcription, said unit containing at least one ablation recognition site; and (b) a second transcription unit that encodes an ablator specific for the at least one ablation recognition site in operative association with a promoter, wherein transcription and/or ablation activity is controlled by a pharmacological agent, e.g., a dimerizer. For example, one suitable pharmacologic agent may be rapamycin or a rapamycin analog. The virus composition may contain two or more different virus stocks.

In one aspect, the invention provides a replication-defective virus composition suitable for use in human subjects in which the viral genome comprises (a) a first transcription unit that encodes a therapeutic product in operative association with a promoter that controls transcription, said first transcription unit containing an ablation recognition site; and a second transcription unit that encodes an ablator specific for the ablation recognition site in operative association with a promoter, wherein transcription and/or ablation activity is controlled by a pharmacological agent. The first transcription unit can contains more than one ablation recognition site. Where the genome comprises more than one ablation recognition site, said more than one ablation recognition site comprising a first ablation recognition site and a second ablation recognition site which differs from said first ablation recognition site, said virus further comprising a first ablator specific for the first ablation recognition site and a second ablator specific for the second recognition site.

In one embodiment, the transcription, bioactivity and/or the DNA binding specificity of the ablator is controlled by a regulatable system. The regulatable system can be selected from a tet-on/off system, a tetR-KRAB system, a mifepristone (RU486) regulatable system, a tamoxifen-dependent regulatable system, a rapamycin-regulatable system, or an ecdysone-based regulatable system.

In one embodiment, the ablator is selected from the group consisting of an endonuclease, a recombinase, a meganuclease, or a zinc finger endonuclease that binds to the ablation recognition site in the first transcription unit and excises or ablates DNA and an interfering RNA, a ribozyme, or an antisense that ablates the RNA transcript of the first transcription unit, or suppresses translation of the RNA transcript of the first transcription unit. In one specific embodiment, the ablator is Cre and the ablation recognition site is loxP, or the ablator is FLP and the ablation recognition site is FRT.

In an embodiment, the ablator is a chimeric engineered endonuclease, wherein the virus composition comprises (i) a first sequence comprising the DNA binding domain of the endonuclease fused to a binding domain for a first pharmacological agent; and wherein the virus composition further comprises (ii) a second sequence encoding the nuclease cleavage domain of the endonuclease fused to a binding domain for the first pharmacological agent, wherein the first sequences (i) and the second sequence (ii) are each in operative association with at least one promoter which controls expression thereof. The chimeric engineered endonuclease can be contained within a single bicistronic open reading frame in the second transcription unit, said transcription unit further comprising a linker between (i) and (ii). Optionally, the sequence (ii) has an inducible promoter. In another embodiment, the fusion partners/fragments of the chimeric engineered endonuclease are contained within separate open reading frames. In one embodiment, each of the first sequence and the second sequence are under the control of a constitutive promoter and the ablator is bioactivated by the first pharmacological agent.

The coding sequence for the ablator may further comprise a nuclear localization signal located 5′ or 3′ to the ablator coding sequence.

In one embodiment, the DNA binding domain is selected from the group consisting of a zinc finger, helix-turn-helix, a HMG-Box, Stat proteins, B3, helix-loop-helix, winged helix-turn-helix, leucine zipper, a winged helix, POU domains, and a homeodomain.

In still another embodiment, the endonuclease is selected from the group consisting of a type II restriction endonuclease, an intron endonuclease, and serine or tyrosine recombinases. In one specific embodiment, the ablator is a chimeric FokI enzyme.

In yet another embodiment, in a replication-defective virus composition of the invention, the viral genome further comprises a third and a fourth transcription unit, each encoding a dimerizable domain of a transcription factor that regulates an inducible promoter for the ablator, in which: (c) the third transcription unit encodes the DNA binding domain of the transcription factor fused to a binding domain for the pharmacological agent in operative association with a first promoter; and (d) the fourth transcription unit encodes the activation domain of the transcription factor fused to a binding domain for the pharmacological agent in operative association with a second promoter. The first promoter of (c) and the second promoter of (d) are independently selected from a constitutive promoter and an inducible promoter. In another embodiment, the first and second promoters are both constitutive promoters and the pharmacological agent is a dimerizer that dimerizes the domains of the transcription factor. In still a further embodiment, one of the first promoter and the second promoters is an inducible promoter. The third and fourth transcription units can be a bicistronic unit containing an IRES or furin-2A.

In one embodiment, the pharmacological agent is rapamycin or a rapalog.

In one embodiment, the virus is an AAV. Such an AAV may be selected from among, e.g., AAV1, AAV6, AAV7, AAV8, AAV9 and rh10. Still other viruses may be used to generate the DNA constructs and replication-defective viruses of the invention including, e.g., adenovirus, herpes simplex viruses, and the like.

In one embodiment, the therapeutic product is an antibody or antibody fragment that neutralizes HIV infectivity, soluble vascular endothelial growth factor receptor-1 (sFlt-1), Factor VIII, Factor IX, insulin like growth factor (IGF), hepatocyte growth factor (HGF), heme oxygenase-1 (HO-1), or nerve growth factor (NGF).

In one embodiment of the replication-defective virus composition, the first transcription unit and the second transcription unit are on different viral stocks in the composition. Optionally, the first transcription unit and the second transcription unit are in a first viral stock and the a second viral stock comprises a second ablator(s).

In one embodiment, a recombinant DNA construct comprises a first and second transcription unit flanked by packaging signals of a viral genome, in which: (a) a first transcription unit that encodes a therapeutic product in operative association with a promoter that controls transcription, said first transcription unit containing at least one ablation recognition site; and (b) a second transcription unit that encodes an ablator specific for the at least one ablation recognition site in operative association with a promoter that induces transcription in response to a pharmacological agent. The packaging signals flanking the transcription units may be an AAV 5′ inverted terminal repeats (ITR) and a AAV 3′ ITR. Optionally, the AAV ITRs are AAV2, or AAV1, AAV6, AAV7, AAV8, AAV9 or rh10 ITRs. In one embodiment, the first transcription unit is flanked by AAV LTRs, and the second, third and fourth transcription units are flanked by AAV ITRs. Optionally, the transcription units are contained in two or more DNA constructs.

In one embodiment, the therapeutic product is an antibody or antibody fragment that neutralizes HIV infectivity, soluble vascular endothelial growth factor receptor-1 (sFlt-1), Factor VIII, Factor IX, insulin like growth factor (IGF), hepatocyte growth factor (HGF), heme oxygenase-1 (HO-1), or nerve growth factor (NGF).

In one embodiment, the promoter that controls transcription of the therapeutic product is a constitutive promoter, a tissue-specific promoter, a cell-specific promoter, an inducible promoter, or a promoter responsive to physiologic cues.

A method is described for treating age-related macular degeneration in a human subject, comprising administering an effective amount of the replication-defective virus composition as described herein, in which the therapeutic product is a VEGF antagonist.

A method is provided for treating hemophilia A in a human subject, comprising administering an effective amount of the replication-defective virus composition as described herein, in which the therapeutic product is Factor VIII.

A method is provided for treating hemophilia B in a human subject, comprising administering an effective amount of the replication-defective virus composition as described herein, in which the therapeutic product is Factor IX.

A method is provided for treating congestive heart failure in a human subject, comprising administering an effective amount of the replication-defective virus composition as described herein, in which the therapeutic product is insulin like growth factor or hepatocyte growth factor.

A method is provided for treating a central nervous system disorder in a human subject, comprising administering an effective amount of the replication-defective virus composition as described herein, in which the therapeutic product is nerve growth factor.

A method is provided for treating HIV infection in a human subject, comprising administering an effective amount of the replication-defective virus composition as described herein in which the therapeutic product is a neutralizing antibody against HIV.

A replication-defective virus is provided herein for use in controlling delivery of the transgene product. The product may be selected from the group consisting of a VEGF antagonist, Factor IX, Factor VIII, insulin like growth factor, hepatocyte growth factor, nerve growth factor, and a neutralizing antibody against HIV.

A genetically engineered cell is provided which comprises a replication-defective virus or a DNA construct as provided herein. The genetically engineered cell may be selected from a plant, bacterial or non-human mammalian cell.

A method is provided for determining when to administer a pharmacological agent for ablating a therapeutic product to a subject who received the replication-defective virus as provided herein containing a therapeutic product and an ablator, comprising: (a) detecting expression of the therapeutic product in a tissue sample obtained from the patient, and (b) detecting a side effect associated with the presence of the therapeutic product in said subject, wherein detection of a side effect associated with the presence of the therapeutic product in said subject indicates a need to administer the pharmacological agent that induces expression of the ablator.

A method is provided for determining when to administer a pharmacological agent for ablating a therapeutic product to a subject who received the replication-defective virus composition as described herein encoding a therapeutic product and an ablator, comprising: detecting the level of a biochemical marker of toxicity associated with the presence of the therapeutic product in a tissue sample obtained from said subject, wherein the level of said marker reflecting toxicity indicates a need to administer the pharmacological agent that induces expression of the ablator.

These methods may further comprise determining the presence of DNA encoding the therapeutic gene product, its RNA transcript, or its encoded protein in a tissue sample from the subject subsequent to treatment with the pharmacological agent that induces expression of the ablator, wherein the presence of the DNA encoding the therapeutic gene product, its RNA transcript, or its encoded protein indicates a need for a repeat treatment with the pharmacological agent that induces expression of the ablator.

The invention further provides a replication-defective virus as described herein for use in controlling delivery of the transgene product.

In another embodiment, the invention provides a genetically engineered cell, comprising a replication-defective virus or a DNA construct as described herein. Such a cell may be a plant, yeast, fungal, insect, bacterial, non-human mammalian cells, or a human cell.

In yet a further embodiment, the invention provides a method of determining when to administer a pharmacological agent for ablating a therapeutic product to a subject who received the replication-defective virus as described herein encoding a therapeutic product and an ablator, comprising: (a) detecting expression of the therapeutic product in a tissue sample obtained from the patient, and (b) detecting a side effect associated with the presence of the therapeutic product in said subject, wherein detection of a side effect associated with the presence of the therapeutic product in said subject indicates a need to administer the pharmacological agent that induces expression of the ablator. In still a further embodiment, the invention provides a method of determining when to administer a pharmacological agent for ablating a therapeutic product to a subject who received the replication-defective virus composition as described herein encoding a therapeutic product and an ablator, comprising: detecting the level of a biochemical marker of toxicity associated with the presence of the therapeutic product in a tissue sample obtained from said subject, wherein the level of said marker reflecting toxicity indicates a need to administer the pharmacological agent that induces expression of the ablator.

Other aspects and advantages of the invention will be readily apparent from the following Detailed Description of the Invention.

As used herein, the following terms will have the indicated meaning:

“Unit” refers to a transcription unit.

“Transgene unit” refers to a DNA that comprises (1) a DNA sequence that encodes a transgene; (2) an ablation recognition site (ARS) contained within or flanking the transgene; and (3) a promoter sequence that regulates expression of the transgene.

“Ablation recognition site” or “ARS” refers to a DNA sequence that (1) can be recognized by the ablator that ablates or excises the transgene from the transgene unit; or (2) encodes an ablation recognition RNA sequence (ARRS)

“Ablation recognition RNA sequence” or “ARRS” refers to an RNA sequence that is recognized by the ablator that ablates the transcription product of the transgene or translation of its mRNA.

“Ablator” refers to any gene product, e.g., translational or transcriptional product, that specifically recognizes/binds to either (a) the ARS of the transgene unit and cleaves or excises the transgene; or (b) the ARRS of the transcribed transgene unit and cleaves or prevents translation of the mRNA transcript.

“Ablation unit” refers to a DNA that comprises (1) a DNA sequence that encodes an Ablator; and (2) a promoter sequence that controls expression of said Ablator.

“Dimerizable transcription factor (TF) domain unit” refers to (1) a DNA sequence that encodes the DNA binding domain of a TF fused to the dimerizer binding domain (DNA binding domain fusion protein) controlled by a promoter; and (2) a DNA sequence that encodes the activation domain of a TF fused to the dimerizer binding domain (activation domain fusion protein) controlled by a promoter. In one embodiment, each unit of the dimerizable domain is controlled by a constitutive promoter and the unit is utilized for control of the promoter for the ablator. Alternatively, one or more of the promoters may be an inducible promoter.

A “Dimerizable fusion protein unit” refers to (1) a first DNA sequence that encodes a unit, subunit or fragment of a protein or enzyme (e.g., an ablator) fused to a dimerizer binding domain and (2) a second DNA sequence that encodes a unit, subunit or fragment of a protein or enzyme, which when expressed and if required, activated, combine to form a fusion protein. This “Dimerizable fusion protein unit” may be utilized for a variety of purposes, including to activate a promoter for the ablator, to provide DNA specificity, to activate a chimeric ablator by bringing together the binding domain and the catalytic domain, or to produce a desired transgene. These units (1) and (2) may be in a single open reading frame separated by a suitable linker (e.g., an IRES or 2A self-cleaving protein) under the control of single promoter, or may be in separate open reading frames under the control of independent promoters. From the following detailed description, it will be apparent that a variety of combinations of constitutive or inducible promoters may be utilized in the two components of this unit, depending upon the use to which this fusion protein unit is put (e.g., for expression of an ablator). In one embodiment, the dimerizable fusion protein unit contains DNA binding domains which include, e.g., zinc finger motifs, homeo domain motifs, HMG-box domains, STAT proteins, B3, helix-loop-helix, winged helix-turn-helix, leucine zipper, helix-turn-helix, winged helix, POU domains, DNA binding domains of repressors, DNA binding domains of oncogenes and naturally occurring sequence-specific DNA binding proteins that recognize >6 base pairs.

“Dimerizer” refers to a compound or other moiety that can bind heterodimerizable binding domains of the TF domain fusion proteins or dimerizable fusion proteins and induce dimerization or oligomerization of the fusion proteins. Typically, the dimerizer is delivered to a subject as a pharmaceutical composition.

“Side effect” refers to an undesirable secondary effect which occurs in a patient in addition to the desired therapeutic effect of a transgene product that was delivered to a patient via administration of a replication-defective virus composition of the invention.

“Replication-defective virus” or “viral vector” refers to a synthetic or artificial genome containing a gene of interest packaged in replication-deficient virus particles; i.e., particles that can infect target cells but cannot generate progeny virions. The artificial genome of the viral vector does not include genes encoding the enzymes required to replicate (the genome can be engineered to be “gutless”—containing only the transgene of interest flanked by the signals required for amplification and packaging of the artificial genome). Therefore, it is deemed safe for use in gene therapy since replication and infection by progeny virions cannot occur except in the presence of the viral enzyme required for replication.

“Virus stocks” or “stocks of replication-defective virus” refers to viral vectors that package the same artificial/synthetic genome (in other words, a homogeneous or clonal population).

A “chimeric engineered ablator” or a “chimeric enzyme” is provided when a sequence encoding a catalytic domain of an endonuclease ablator fused to a binding domain and a sequence encoding a DNA binding domain of the endonuclease fused to a binding domain are co-expressed. The chimeric engineered enzyme is a dimer, the DNA binding domains may be selected from among, for example, zinc finger and other homeodomain motifs, HMG-box domains, STAT proteins, B3, helix-loop-helix, winged helix-turn-helix, leucine zipper, helix-turn-helix, winged helix, POU domains, DNA binding domains of repressors, DNA binding domains of oncogenes and naturally occurring sequence-specific DNA binding proteins that recognize >6 base pairs. [U.S. Pat. No. 5,436,150, issued Jul. 25, 1995]. When a heterodimer is formed, the binding domains are specific for a pharmacologic agent that induces dimerization in order to provide the desired enzymatic bioactivity, DNA binding specificity, and/or transcription of the ablator. Typically, an enzyme is selected which has dual domains, i.e., a catalytic domain and a DNA binding domain which are readily separable. In one embodiment, a type II restriction endonuclease is selected. In one embodiment, a chimeric endonuclease is designed based on an endonuclease having two functional domains, which are independent of ATP hydrolysis. Useful nucleases include type II S endonucleases such as FokI, or an endonuclease such as Nae I. Another suitable endonuclease may be selected from among intron endonucleases, such as e.g., I-TevI. Still other suitable nucleases include, e.g., integrases (catalyze integration), serine recombinases (catalyze recombination), tyrosine recombinases, invertases (e.g. Gin) (catalyze inversion), resolvases, (e.g., Tn3), and nucleases that catalyze translocation, resolution, insertion, deletion, degradation or exchange. However, other suitable nucleases may be selected.

### 4. BRIEF DESCRIPTION OF DRAWINGS

FIGS. 1A-1D. Comparison of transfection agents for rAAV7 productivity and release to the culture medium. FIGS. 1A-1B: 6 well plates were seeded with HEK 293 cells and transfected with three plasmids (carrying the vector genome, AAV2 rep/AAV7 cap genes, and adenovirus helper functions, respectively) using calcium phosphate (FIG. 1A) or polyethylenimine (PEI) (FIG. 1B) as the transfection reagent. DNase resistant vector genome copies (GC) present in cell lysates and the production culture medium at 72 hours post-transfection were quantified by qPCR. FIGS. 1C and 1D: 10 layer Corning cell stacks containing HEK (293 cells were triple transfected by both calcium phosphate (FIG. 1C) or PEI (FIG. 1D) methods and vector GC in the culture supernatant and cells was determined 120 hours later.

FIG. 2. Productivity and release of different serotypes following PET transfection in the presence or absence of 500 mM salt. 15 cm plates of HEK 293 cells were triple transfected using PEI and DNA mixes containing one of the 5 different AAV capsid genes indicated. 5 days post-transfection, culture medium and cells were harvested either with or without exposure to 0.5 M salt and the DNase resistant vector genome copies (GC) quantified. GC produced per cell are represented with the percentage of vector found in the supernatant indicated above each bar.

FIGS. 3A-3B. Large scale iodixanol gradient-based purification of rAAV7 vector from concentrated production culture supernatants. FIG. 3A: rAAV7 vector from cell stack culture medium was concentrated and separated on iodixanol gradients and fractions harvested from the bottom of the tube (fraction 1). Iodixanol density was monitored at 340 nm and genome copy numbers for each fraction was obtained by qPCR. FIG. 3B: 1×10¹⁰ GC of each fraction was analyzed by SDS-PAGE and proteins visualized using sypro ruby stain. V=validation lot; M=molecular weight marker. The AAV capsid proteins VP1, VP2 and VP3 are indicated. The pure AAV vector peak is indicated by the white box on the SDS-PAGE gel.

FIG. 4. Purity of large scale rAAV production lots. 1×10¹⁰ GC of large scale AAV8 and AAV9 vector preparations were loaded to SDS-PAGE gels and proteins were visualized by sypro ruby staining. All protein bands were quantified and the percent purity of the capsid (VP1, VP2 and VP3 proteins indicated over total protein) was calculated and indicated below the gel. The purity of the large scale lots were compared with a small scale CsCl gradient purified AAV9 vector.

FIGS. 5 A-G. Determination of empty-to-full particle ratios in large scale rAAV8 and rAAV9 production lots. Large scale rAAV8 and rAAV9 vector preparations were negatively stained with uranyl acetate and examined with a transmission electron microscope. FIG. 5A is pilot run 1. FIG. 5B is pilot run 8. FIG. 5C is pilot run 9. FIG. 5D is pilot run 10. FIG. 5E is pilot run 11. FIG. 5F is pilot run 12. Empty particles are distinguished based on the electron-dense center and are indicated by arrows. The ratio of empty-to-full particles and the percentage of empty particles are shown below the images. FIG. 5G is the small scale AAV8 vector prep included in the analysis for comparison.

FIGS. 6A-6G. Relative transduction of rAAV8, rAAV9 and rAAV6 vectors in vitro. FIG. 6A-F: HEK 293 cells were infected in triplicate with rAAV-eGFP vector lots produced by both large and small scale processes at an MOI of 1×10⁴ GC/cell in the presence of adenovirus. GFP transgene expression was photographed at 48 hrs PI. FIG. 6G: eGFP fluorescence intensity was quantified directly from the digital images by determining the product of brightness levels and pixels over background levels.

FIGS. 7A-7G. Liver transduction of rAAV8 and rAAV9 large scale production lots. FIGS. 7A-7F: C57BL/6 mice were injected i.v. with 1×10¹¹ GC rAAV8-eGFP and rAAV9-eGFP vectors produced by both small and large scale processes. FIG. 7A is pilot run 1 for AAV9, FIG. 7B is pilot run 9 for AAV9, and FIG. 7C is CsCl (small scale) for AAV9. FIG. 7D is pilot run 10 for AAV8. FIG. 7E is pilot run 12 for AAV8 and FIG. 7F is CsCl (small scale) for AAV8. eGFP fluorescence was compared in liver sections at 9 days post-injection. FIG. 7G: eGFP fluorescence intensity was quantified directly from the digital images by determining the product of brightness levels and pixels over background levels. Each bar represents the average intensity value of liver samples from two animals.

FIGS. 8A and 8B. PITA DNA construct containing a dimerizable transcription factor domain unit and an ablation unit. FIG. 8A is a map of the following DNA construct, which comprises a dimerizable transcription factor domain unit and an ablation unit: pAAV.CMV.TF.FRB-IRES-1xFKBP.Cre. FIG. 8B is a cartoon of the transcription unit inserted into the plasmid backbone. A description of the various vector domains can be found in Section 8.1 herein.

FIGS. 9A and 9B. PITA DNA construct containing a dimerizable transcription factor domain unit and an ablation unit. FIG. 9A is a map of the following DNA construct, which comprises a dimerizable transcription factor domain unit and an ablation unit: pAAV.CMV.TF.FRB-T2A-2xFKBP.Cre. FIG. 9B is a cartoon of the transcription unit inserted into the plasmid backbone. A description of the various vector domains can be found in Section 8.1 herein.

FIGS. 10A and 10B. PITA DNA construct containing a dimerizable transcription factor domain unit and an ablation unit. FIG. 10A is map of the following DNA construct, which comprises a dimerizable transcription factor domain unit and an ablation unit: pAAV.CMV173.TF.FRB-T2A-3xFKBP.Cre. FIG. 10B is a cartoon of the transcription unit inserted into the plasmid backbone. A description of the various vector domains can be found in Section 8.1 herein.

FIGS. 11A and 11B. PITA DNA construct containing a dimerizable transcription factor domain unit and an ablation unit. FIG. 11A is a map of the following DNA construct, which comprises a dimerizable transcription factor domain unit and an ablation unit: pAAV.CMV.TF.FRB-T2A-2xFKBP.ISce-I. FIG. 11B is a cartoon of the transcription unit inserted into the plasmid backbone. A description of the various vector domains can be found in Section 8.1 herein.

FIGS. 12A and 12B. PITA DNA construct containing a transgene unit. FIG. 12A is a map of the following DNA construct, which comprises a transgene unit: pENN.CMV.PLloxP.Luc.SV40. FIG. 12B is a cartoon of the transcription unit inserted into the plasmid backbone. A description of the various vector domains can be found in Section 8.2 herein.

FIGS. 13A and 13B. PITA DNA construct containing a transgene unit. FIG. 13A is a map of the following DNA construct, which comprises a transgene unit: pENN.CMV.PISceI.UC.SV40. FIG. 13B is a cartoon of the transcription unit inserted into the plasmid backbone. A description of the various vector domains can be found in Section 8.2 herein.

FIG. 14. PITA DNA construct containing a dimerizable transcription factor domain unit and a transgene unit. FIG. 14 is a map of a vector that contains a transgene unit and a dimerizable transcription factor domain unit. A description of the various vector domains can be found in Sections 8.1 and 8.2 herein.

FIGS. 15A-B. In vitro induction of luciferase after rapamycin treatment. FIG. 15A is a bar graph showing relative luciferase activity in cells that were transfected with the indicated DNA constructs (DNA constructs 1 to 6) 48 hours after either being treated or not treated with rapamycin. FIG. 15B is a bar graph showing relative luciferase activity in cells that were transfected with the indicated DNA constructs (DNA constructs 1 to 6) 72 hours after either being treated or not treated with rapamycin.

FIGS. 16A-D. In the in vivo model for a dimerizer-inducible system, four groups of mice received IV injection of AAV vectors containing the following DNA constructs. FIG. 16A is a diagram of a DNA construct encoding GFP-Luciferase under the control of ubiquitous constitutive CMV promoter, which was delivered to Group 1 mice via AAV vectors. FIG. 168 is a diagram of DNA constructs encoding (1) a dimerizable transcription factor domain unit (FRB fused with p65 activation domain and DNA binding domain ZFHD fused with 3 copies of FKBP) driven by the CMV promoter; and (2) AAV vector expressing GFP-Luciferase driven by a promoter induced by the dimerized TF, which were delivered to Group 2 mice via AAV vectors. FIG. 16C is a diagram of a DNA construct encoding GFP-Luciferase under the control of a liver constitutive promoter, TBG, which was delivered to Group 3 mice via AAV vectors. FIG. 16D is a diagram of DNA constructs encoding (1) AAV vector expressing a dimerizable transcription factor domain unit driven by the TBG promoter; and (2) AAV vector expressing GFP-Luciferase driven by a promoter induced by the dimerized TF, which were delivered to Group 4 mice via AAV vectors.

FIGS. 17 A-D. Image of 4 groups of mice that received 3×10¹¹ particles of AAV virus containing various DNA constructs 30 minutes after injection of luciferin, the substrate for luciferase. FIG. 17A shows luciferase expression in various tissues, predominantly in lungs, liver and muscle, in Group 1 mice before (“Pre”) and after (“Post”) rapamycin administration. FIG. 17B shows luciferase expression, predominantly in liver and muscle in Group 2 mice before (“Pre”) and after (“Post”) rapamycin administration. FIG. 17C shows luciferase expression predominantly in liver and muscle after (“Post”) rapamycin administration, and shows that there is no luciferase expression before (“Pre”) rapamycin administration in Group 3 mice. FIG. 17D shows luciferase expression is restricted to the liver (“Post”) rapamycin administration and shows that there is no luciferase expression before (“Pre”) rapamycin administration.

FIGS. 18 A-D. Image of 4 groups of mice that received 1×10¹¹ particles of AAV virus containing various DNA constructs 30 minutes after injection of luciferin, the substrate for luciferase. FIG. 18A shows luciferase expression in various tissues, predominantly in lungs, liver and muscle, in Group 1 mice before (“Pre”) and after (“Post”) rapamycin administration. FIG. 18B shows luciferase expression, predominantly in liver and muscle in Group 2 mice before (“Pre”) and after (“Post”) rapamycin administration. FIG. 18C shows luciferase expression predominantly in liver and muscle after (“Post”) rapamycin administration, and shows that there is no luciferase expression before (“Pre”) rapamycin administration in Group 3 mice. FIG. 18D shows luciferase expression is restricted to the liver (“Post”) rapamycin administration and shows that there is no luciferase expression before (“Pre”) rapamycin administration.

FIGS. 19 A-C. PITA DNA constructs for treating AMD. FIG. 19A shows a DNA construct comprising a transgene unit that encodes a soluble VEGF receptor, sFlt-1. FIG. 19B shows a bicistronic DNA construct comprising Avastin IgG heavy chain (AvastinH) and light chain (AvastinL) regulated by IRES. FIG. 19C shows a bicistronic DNA construct comprising Avastin IgG heavy chain (AvastinH) and light chain (AvastinL) separated by a T2A sequence.

FIGS. 20 A-B. PITA DNA constructs for treating Liver Metabolic Disease. FIG. 20A shows a PITA DNA construct for treating hemophilia A and/or B, containing a transgene unit comprising Factor IX. FIG. 20B shows a DNA construct for delivery of shRNA targeting the IRES of HCV.

FIGS. 21A-B. PITA DNA constructs for treating Heart Disease. FIG. 21A shows a PITA DNA construct for treating congestive heart failure, containing a transgene unit comprising insulin like growth factor (IGFI). FIG. 21B shows a PITA DNA construct for treating congestive heart failure, containing a transgene unit comprising hepatocyte growth factor (HGF).

FIG. 22. PITA DNA construct for a CNS disease. FIG. 22 shows a PITA DNA construct for treating Alzheimer's disease, containing a transgene unit comprising nerve growth factor (NGF).

FIG. 23. PITA System for HIV treatment. FIG. 23 shows a PITA DNA construct containing a transgene unit comprising the heavy and light chains of an HIV antibody and a PITA DNA construct containing an ablation unit and a dimerizable TF domain unit. FIG. 23 also shows that a rapamycin analog (rapalog) can induce expression of the ablator, cre, to ablate the transgene (heavy and light chains of an HIV antibody) from the PITA DNA construct containing a transgene unit.

FIG. 24. Illustration of one embodiment of the PITA system. FIG. 24 shows a transgene unit encoding a therapeutic antibody that is in operative association with a constitutive promoter, an ablation unit encoding an endonuclease that is in operative association with a transcription factor inducible promoter, and a dimerizable TF domain unit, with each transcription factor domain fusion sequence in operative association with a constitutive promoter. Prior to administration of rapamycin or a rapalog, there is baseline expression of the therapeutic antibody and of the two transcription factor domain fusion proteins. Upon rapamycin administration, the dimerized transcription factor induces expression of the endonuclease, which cleaves the endonuclease recognition domain in the transgene unit, thereby ablating transgene expression.

FIGS. 25A-25B are bar charts illustrating that wild-type Fold effective ablated expression of a transgene when a DNA plasmid containing a transgene containing ablation sites for FokI was cotransfected into target cells with a plasmid encoding the Fold enzyme. FIG. 25A, bar 1 represents 50 ng pCMV.Luciferase, bar 2 represents 50 ng pCMV.Luciferase+200 ng pCMV.FokI, bar 3 represents 50 ng pCMV.Luciferase+transfected FokI protein, bar 4 represents transfected FokI protein alone; bar 5 represents untransfected controls. FIG. 25B, bar 1 represents 50 ng pCMV.Luc alone, subsequent bars represent increasing concentrations of a ZFHD-FokI expression plasmid (6.25, 12.5, 25, 50, and 100 ng) cotransfected with pCMV.Luciferase. This study is described in Example 11A.

FIGS. 26A-B are bar charts illustrating that a chimeric engineered enzyme tethered to a non-cognate recognition site on the DNA by the zinc finger homeodomain effectively ablates expression of a transgene. FIG. 26A compares increasing concentrations of an expression plasmid encoding un-tethered Fold (6.25 ng, 12.5 ng, 25 ng, 50 ng and 100 ng) co-transfected with pCMV.luciferase. The first bar provides a positive control of 50 ng pCMV.Luc alone. FIG. 26B compares increasing concentrations of an expression plasmid encoding FokI tethered to DNA via fusion with the zinc finger homeodomain (6.25 ng, 12.5 ng, 25 ng, 50 ng and 100 ng) co-transfected with pCMV.luciferase. The first bar provides a control of 50 ng pCMV.Luc alone. This study is described in Example 11B.

FIGS. 27A-B are bar charts illustrating that the DNA binding specificity of chimeric FokI can be reproducible changed by fusion with various classes of heterologous DNA binding domains and ablation of target transgene can be further improved by the additional of a heterologous nuclear localization signal (NLS). FIG. 27A illustrates the results of co-transfection of pCMV.Luciferase with increasing concentrations of an expression plasmid encoding FokI tethered to DNA via an HTH fusion (6.25, 12.5, 25, 50, and 100 ng). The first bar is a control showing 50 ng pCMV.Luciferase alone. FIG. 27B illustrates the results of co-transfection of pCMV.Luciferase with increasing concentrations of an expression plasmid encoding an HTH Fold fusion, which further has a NLS at its N-terminus (6.25, 115, 25, 50, and 100 ng). The first bar is a control showing 50 ng pCMV.Luciferase alone. This study is described in Example 11C.

### 5. DETAILED DESCRIPTION OF THE INVENTION

In the PITA system, one or more replication-defective viruses are used in a replication-defective virus composition in which the viral genome(s) have been engineered to contain: (a) a first transcription unit that encodes a therapeutic product in operative association with a promoter that controls transcription, said unit containing at least one ablation recognition site; and (b) a second transcription unit that encodes an ablator (or a fragment thereof as part of a fusion protein unit) specific for the ablation recognition site in operative association with a promoter that induces transcription in response to a pharmacological agent. Any pharmacological agent that specifically dimerizes the domains of the selected binding domain can be used. In one embodiment, rapamycin and its analogs referred to as “rapalogs” can be used.

A viral genome containing a first transcription unit may contain two or more of the same ablation recognition site or two or more different ablation recognition sites (i.e., which are specific sites for a different ablator than that which recognizes the other ablation recognition site(s)). Whether the same or different, such two or more ablation recognition sites may be located in tandem to one another, or may be located in a position non-contiguous to the other. Further, the ablation recognition site(s) may be located at any position relative the coding sequence for the transgene, i.e., within the transgene coding sequence, 5′ to the coding sequence (either immediately 5′ or separated by one or more bases, e.g., upstream or downstream of the promoter) or 3′ to the coding sequence (e.g., either immediately 3′ or separated by one or more bases, e.g., upstream of the poly A sequence).

An ablator is any gene product, e.g., translational or transcriptional product, that specifically recognizes/binds to either (a) the ablation recognition site(s) (ARS) of the transgene unit and cleaves or excises the transgene; or (b) the ablation recognition RNA sequence (ARRS) of the transcribed transgene unit and cleaves or inhibits translation of the mRNA transcript. As described herein, an ablator may be selected from the group consisting of: an endonuclease, a recombinase, a meganuclease, or a zinc finger endonuclease that binds to the ablation recognition site in the first transcription unit and excises or ablates DNA and an interfering RNA, a ribozyme, or an antisense that ablates the RNA transcript of the first transcription unit, or suppresses translation of the RNA transcript of the first transcription unit. In one specific embodiment, the ablator is Cre (which has as its ablation recognition site loxP), or the ablator is FLP (which has as its ablation recognition site FRT). In one embodiment, an endonuclease is selected which functions independently of ATP hydrolysis. Examples of such ablators may include a Type II S endonuclease (e.g., FokI), NaeI, and intron endonucleases (such as e.g., 1-TevI), integrases (catalyze integration), serine recombinases (catalyze recombination), tyrosine recombinases, invertases (e.g. Gin) (catalyze inversion), resolvases, (e.g., Tn3), and nucleases that catalyze translocation, resolution, insertion, deletion, degradation or exchange. However, other suitable nucleases may be selected.

For permanent shut down of the therapeutic transgene, the ablator can be an endonuclease that binds to the ablation recognition site(s) in the first transcription unit and ablates or excises the transgene. Where temporary shutdown of the transgene is desired, an ablator should be chosen that binds to the ablation recognition site(s) in the RNA transcript of the therapeutic transgene and ablates the transcript, or inhibits its translation. In this case, interfering RNAs, ribozymes, or antisense systems can be used. The system is particularly desirable if the therapeutic transgene is administered to treat cancer, a variety of genetic disease which will be readily apparent to one of skill in the art, or to mediate host immune response.

Expression of the ablator may be controlled by one or more elements, including, e.g., an inducible promoter and/or by use of a chimeric ablator that utilizes a homodimer or heterodimer fusion protein system, such as are described herein. Where use of a homodimer system is selected, expression of the ablator is controlled by an inducible promoter. Where use of heterodimer system is selected, expression of the ablator is controlled by additional of a pharmacologic agent and optionally, a further inducible promoter for one or both of the fusion proteins which form the heterodimer system. In one embodiment, a homo- and hetero-dimizerable ablator is selected to provide an additional layer for safety to constructs with transcription factor regulators. These systems are described in more detail later in this specification.

Any virus suitable for gene therapy may be used, including but not limited to adeno-associated virus (“AAV”); adenovirus; herpes virus; lentivirus; retrovirus; etc. In preferred embodiments, the replication-defective virus used is an adeno-associated virus (“AAV”). AAV1, AAV6, AAV7, AAV8, AAV9 or rh10 being particularly attractive for use in human subjects. Due to size constraints of the AAV genome for packaging, the transcription units can be engineered and packaged in two or more AAV stocks. Whether packaged in one viral stock which is used as a virus composition according to the invention, or in two or more viral stocks which form a virus composition of the invention, the viral genome used for treatment must collectively contain the first and second transcription units encoding the therapeutic transgene and the ablator; and may further comprise additional transcription units. For example, the first transcription unit can be packaged in one viral stock, and second, third and fourth transcription units packaged in a second viral stock. Alternatively, the second transcription unit can be packaged in one viral stock, and the first, third and fourth transcription units packaged in a second viral stock. While useful for AAV due to size contains in packaging the AAV genome, other viruses may be used to prepare a virus composition according to the invention. In another embodiment, the viral compositions of the invention, where they contain multiple viruses, may contain different replication-defective viruses (e.g., AAV and adenovirus).

In one embodiment, a virus composition according to the invention contains two or more different AAV (or another viral) stock, in such combinations as are described above. For example, a virus composition may contain a first viral stock comprising the therapeutic gene with ablator recognition sites and a first ablator and a second viral stock containing an additional ablator(s). Another viral composition may contain a first virus stock comprising a therapeutic gene and a fragment of an ablator and a second virus stock comprising another fragment of an ablator. Various other combinations of two or more viral stocks in a virus composition of the invention will be apparent from the description of the components of the present system.

In order to conserve space within the viral genome(s), bicistronic transcription units can be engineered. For example, transcription units that can be regulated by the same promoter, e.g., the third and fourth transcription units (and where applicable, the first transcription unit encoding the therapeutic transgene) can be engineered as a bicistronic unit containing an IRES (internal ribosome entry site) or a 2A peptide, which self-cleaves in a post-translational event (e.g., furin-2A), and which allows coexpression of heterologous gene products by a message from a single promoter when the transgene (or an ablator coding sequence) is large, consists of multi-subunits, or two transgenes are co-delivered, recombinant AAV (rAAV) carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study. A single promoter may direct expression of an RNA that contains, in a single open reading frame (ORF), two or three heterologous genes (e.g., the third and fourth transcription units, and where applicable, the first transcription unit encoding the therapeutic transgene) separated from one another by sequences encoding a self-cleavage peptide (e.g., 2A peptide, T2A) or a protease recognition site (e.g., furin). The ORF thus encodes a single polyprotein, which, either during (in the case of T2A) or after translation, is cleaved into the individual proteins. These IRES and polyprotein systems can be used to save AAV packaging space, they can only be used for expression of components that can be driven by the same promoter.

The invention also relates to DNA constructs used to engineer cell lines for the production of the replication-defective virus compositions; methods for producing and manufacturing the replication-defective virus compositions; expression in a variety of cell types and systems, including plants, bacteria, mammalian cells, etc., and methods of treatment using the replication-defective virus compositions for gene transfer, including veterinary treatment (e.g., in livestock and other mammals), and for in vivo or ex vivo therapy, including gene therapy in human subjects.

### 5.1. Transgene Ablation System

The present invention provides a Pharmacologically Induced Transgene Ablation (PITA) System designed for the delivery of a transgene (encoding a therapeutic product—protein or RNA) using replication-defective virus compositions engineered with a built-in safety mechanism for ablating the therapeutic gene product, either permanently or temporarily, in response to a pharmacological agent—preferably an oral formulation, e.g., a pill containing a small molecule that induces expression of the ablator specific for the transgene or its transcription product. However, other routes of delivery for the pharmacologic agent may be selected.

In the PITA system, one or more replication-defective viruses are used in which the viral genome(s) have been engineered to contain a transgene unit (described in Section 5.1.1 herein) and an ablation unit (described in Section 5.1.2 herein). In particular, one or more replication-defective viruses are used in which the viral genome(s) have been engineered to contain (a) a first transcription unit that encodes a therapeutic product in operative association with a promoter that controls transcription, said unit containing at least one ablation recognition site (a transgene unit); and (b) a second transcription unit that encodes an ablator specific for the ablation recognition site in operative association with a promoter that induces transcription in response to a pharmacological agent (an ablation unit).

In one embodiment, the PITA system is designed such that the viral genome(s) of the replication-defective viruses are further engineered to contain a dimerizable domain unit (described in Section 5.1.3). In one embodiment, by delivering a dimerizable TF domain unit, target cells are modified to co-express two fusion proteins: one containing a DNA-binding domain (DBD) of the transcription factor that binds the inducible promoter controlling the ablator and the other containing a transcriptional activation domain (AD) of the transcription factor that activates the inducible promoter controlling the ablator, each fused to dimerizer binding domains (described in Section 5.1.3). Addition of a pharmacological agent, or “dimerizer” (described in Section 5.1.4) that can simultaneously interact with the dimerizer binding domains present in both fusion proteins results in recruitment of the AD fusion protein to the regulated promoter, initiating transcription of the ablator. See, e.g., the Ariad ARGENT® system described in U.S. Pat. No. 5,834,266 and U.S. Pat. No. 7,109,317, each of which is incorporated by reference herein in its entirety. By using dimerizer binding domains that have no affinity for one another in the absence of ligand and an appropriate minimal promoter, transcription is made absolutely dependent on the addition of the dimerizer.

To this end, the viral genome(s) of the replication-defective viruses can be further engineered to contain a third and a fourth transcription unit (a dimerizable TF domain unit), each encoding a dimerizable domain of a transcription factor that regulates the inducible promoter of the ablator in second transcription unit, in which: (c) the third transcription unit encodes the DNA binding domain of the transcription factor fused to a binding domain for the pharmacological agent in operative association with a constitutive promoter; and (d) the fourth transcription unit encodes the activation domain of the transcription factor fused to a binding domain for the pharmacological agent in operative association with a promoter. In one embodiment, each component of the dimerizable TF domain is expressed under constitutive promoter. In another embodiment, at least one component of the dimerizable TF domain unit is expressed under an inducible promoter.

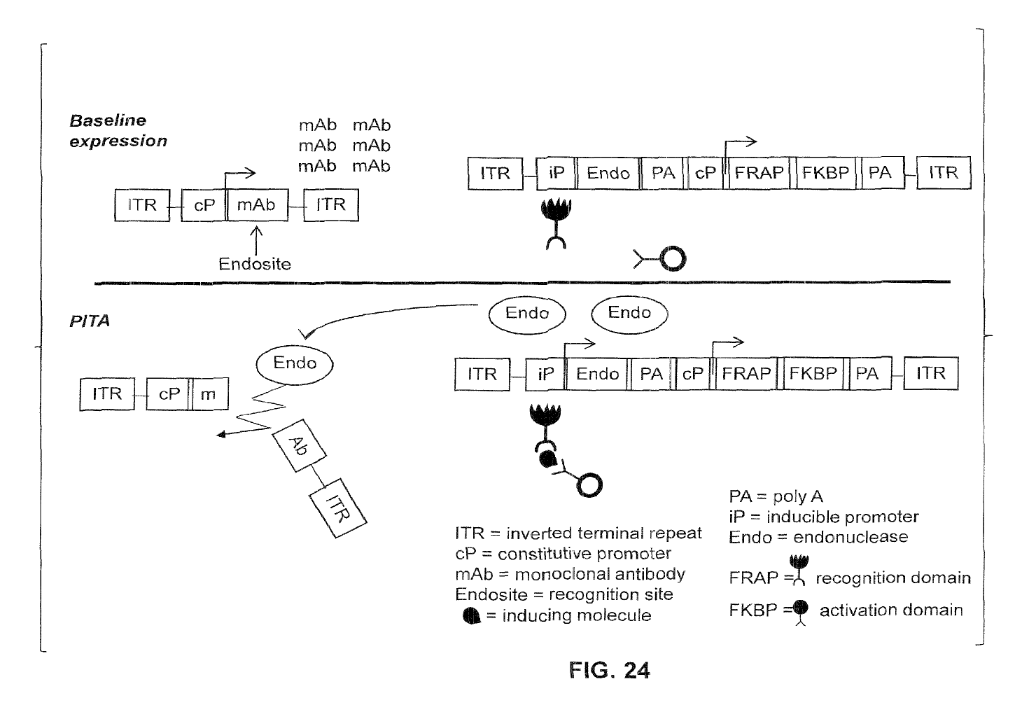

One embodiment of the PITA system is illustrated in FIG. 24, which shows a transgene unit encoding a therapeutic antibody that is in operative association with a constitutive promoter, an ablation unit encoding an endonuclease that is in operative association with a transcription factor inducible promoter, and a dimerizable TF domain unit, with each transcription factor domain fusion sequence in operative association with a constitutive promoter. Prior to administration of rapamycin or a rapalog, there is baseline expression of the therapeutic antibody and of the two transcription factor domain fusion proteins. Upon rapamycin administration, the dimerized transcription factor induces expression of the endonuclease, which cleaves the endonuclease recognition domain in the transgene unit, thereby ablating transgene expression.

In one embodiment, the replication-defective virus used in the PITA system is an adeno-associated virus (“AAV”) (described in Section 5.1.5). AAV1, AAV6, AAV7, AAV8, AAV9 or rh10 are particularly attractive for use in human subjects. Due to size constraints of the AAV genome for packaging, the transcription units can be engineered and packaged in two or more AAV stocks. For example, the first transcription unit can be packaged in one AAV stock, and the second, third and fourth transcription units packaged in a second AAV stock. Alternatively, the second transcription unit can be packaged in one AAV stock, and the first, third and fourth transcription units packaged in a second AAV stock.

5.1.1. Transgene Unit

In the PITA system, one or more replication-defective viruses are used in which the viral genome(s) have been engineered to contain a transgene unit. As used herein, the term “transgene unit” refers to a DNA that comprises: (1) a DNA sequence that encodes a transgene; (2) at least one ablation recognition site (ARS) contained in a location which disrupts transgene expression, including, within or flanking the transgene or its expression control elements (e.g., upstream or downstream of the promoter and/or upstream of the polyA signal); and (3) a promoter sequence that regulates expression of the transgene. The DNA encoding the transgene can be genomic DNA, cDNA, or a cDNA that includes one or more introns which e.g., may enhance expression of the transgene. In systems designed for removal of the transgene, the ARS used is one recognized by the ablator (described in Section 5.1.2) that ablates or excises the transgene, e.g., an endonuclease recognition sequence including but not limited to a recombinase (e.g., the Cre/loxP system, the FLP/FRT system), a meganuclease (e.g., I-SceI system), an artificial restriction enzyme system or another artificial restriction enzyme system, such as the zinc finger nuclease, or a restriction enzyme specific for a restriction site that occurs rarely in the human genome, and the like. To repress expression of the transgene, the ARS can encode an ablation recognition RNA sequence (ARKS), i.e., an RNA sequence recognized by the ablator that ablates the transcription product of the transgene or translation of its mRNA, e.g., a ribozyme recognition sequence, an RNAi recognition sequence, or an antisense recognition sequence.

Examples of transgenes that can be engineered in the transgene units of the present invention includes, but are not limited to a transgene that encodes: an antibody or antibody fragment that neutralizes HIV infectivity, a therapeutic antibody such as VEGF antibody, TNF-α antibody (e.g., infliximab, adalimumab), an EGF-R antibody, basiliximab, cetuximab, infliximab, rituxumab, alemtuzumab-CLL, daclizumab, efalizumab, omalizumab, pavilizumab, trastuzumab, gemtuzumab, adalimumab, or an antibody fragment of any of the foregoing therapeutic antibodies; soluble vascular endothelial growth factor receptor-1 (sFIt-1), soluble TNF-a receptor (e.g., etanercept), Factor VIII, Factor IX, insulin, insulin like growth factor (IGF), hepatocyte growth factor (RGF), heme oxygenase-1 (RO-1), nerve growth factor (NGF), beta-IFN, IL-6, anti-EGFR antibody, interferon (IFN), IFN beta-1 a, anti-CD20 antibody, glucagon-like peptide-1 (GLP-1), anti-cellular adhesion molecule, a4-integrin antibody, glial cell line-derived neurotrophic factor (GDNF), aromatic L-amino acid decarboxylase (ADCC), brain-derived neurotrophic factor (BDNF), ciliary neurotrophic factor (CNTF), galanin, neuropeptide Y (NPY), a TNF antagonist, chemokines from the IL-8 family, BCl2, IL-10, a therapeutic siRNA, a therapeutic u6 protein, endostatin, plasminogen or a fragment thereof, TIMP3, VEGF-A, RIFI alpha, PEDF, or IL-1 receptor antagonist.

The transgene can be under the control of a constitutive promoter, an inducible promoter, a tissue-specific promoter, or a promoter regulated by physiological cues.

Examples of constitutive promoters suitable for controlling expression of the therapeutic products include, but are not limited to human cytomegalovirus (CMV) promoter, the early and late promoters of simian virus 40 (SV40), U6 promoter, metallothionein promoters, EF1a promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86: 10006-10010 (1989>>, the long terminal repeats (LTR) of Moloney Leukemia Virus and other retroviruses, the thymidine kinase promoter of Herpes Simplex Virus and other constitutive promoters known to those of skill in the art.

Inducible promoters suitable for controlling expression of the therapeutic product include promoters responsive to exogenous agents (e.g., pharmacological agents) or to physiological cues. These response elements include, but are not limited to a hypoxia response element (HRE) that binds HIF-1α and β, tetracycline response element (such as described by Gossen & Bujard (1992, Proc. Natl. Acad. Sci. USA 89:5547-551); an ecdysone-inducible response element (No D et al., 1996, Proc. Natl. Acad. Sci. USA. 93:3346-3351) a metal-ion response element such as described by Mayo et al. (1982, Cell 29:99-108); Brinster et al. (1982, Nature 296:39-42) and Searle et al. (1985, Mol. Cell. Biol. 5:1480-1489); a heat shock response element such as described by Nouer et al. (in: Heat Shock Response, ed. Nouer, L., CRC, Boca Raton, Fla., ppI67-220, 1991); or a hormone response element such as described by Lee et al. (1981, Nature 294:228-232); Hynes et al. (Proc. Natl. Acad. Sci. USA 78:2038-2042, 1981); Klock et al. (Nature 329:734-736, 1987); and Israel and Kaufman (1989, Nucl. Acids Res. 17:2589-2604) and other inducible promoters known in the art. Preferably the response element is an ecdysone-inducible response element, more preferably the response element is a tetracycline response element.

Examples of tissue-specific promoters suitable for use in the present invention include, but are not limited to those listed in Table 1 and other tissue-specific promoters known in the art.

TABLE 1
Tissue-specific promoters

| Tissue | Promoter |
|---|---|
| Liver | TBG, A1AT |
| Heart | Troponin T (TnT) |
| Lung | CC10, SPC, FoxJ1 |
| Central Nervous | Synapsin, Tyrosine Hydroxylase, |
| System/Brain | CaMKII (Ca2+/calmodulin- |
| | dependent protein kinase) |
| Pancreas | Insulin, Elastase-I |
| Adipocyte | Ap2, Adiponectin |
| Muscle | Desmin, MHC |
| Endothelial cells | Endothelin-I (ET-I), Flt-I |
| Retina | VMD |

For example, and not by way of limitation, the replication-defective virus compositions of the invention can be used to deliver a VEGF antagonist for treating accelerated macular degeneration in a human subject; Factor VIII for treating hemophilia A in a human subject; Factor IX for treating hemophilia B in a human subject; insulin like growth factor (IGF) or hepatocyte growth factor (HGF) for treating congestive heart failure in a human subject; nerve growth factor (NGF) for treating a central nervous system disorder in a human subject; or a neutralizing antibody against HIV for treating HIV infection in a human subject.

Still other useful therapeutic products include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-25 (including, e.g., IL-2, IL-4, IL-12 and IL-18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors. Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)).

5.1.2. Ablation Unit

The viral genome(s) of one or more replication-defective viruses used in the PITA system are engineered to further contain an ablation unit or coding sequences for an ablator, as defined here.

For permanent shut down of transgene expression, the ablator can be an endonuclease, including but not limited to a recombinase, a meganuclease, a zinc finger endonuclease or any restriction enzyme with a restriction site that rarely occurs in the human genome, that binds to the ARS of the transgene unit and ablates or excises the transgene. Examples of such ablators include, but are not limited to the Cre/loxP system (Groth et al., 2000, Proc. Natl. Acad. Sci. USA 97, 5995-6000); the FLP/FRT system (Sorrell et al., 2005, Biotechnol. Adv. 23, 431-469); meganucleases such as I-SceI which recognizes a specific asymmetric 18 bp element (T AGGGAT AACAGGGT AAT (SEQ ID NO: 25)), a rare sequence in the mammalian genome, and creates double strand breaks (Jasin, M., 1996, Trends Genet., 12, 224-228); and artificial restriction enzymes (e.g., a zinc finger nucleases generated by fusing a zinc finger DNA-binding domain to a DNA-cleavage domain that can be engineered to target ARS sequences unique to the mammalian genome (Miller et al., 2008, Proc. Natl. Acad. Sci. USA, 105: 5809-5814)). In one embodiment, the ablator is a chimeric enzyme, which may be based on a homodimer or a heterodimer fusion protein.

Where temporary shutdown of the transgene is desired, an ablator should be chosen that binds to the ARRS of the RNA transcript of the transgene unit and ablates the transcript, or inhibits its translation. Examples of such ablators include, but are not limited to interfering RNAs (RNAi), ribozymes such as riboswitch (Bayer et al., 2005, Nat Biotechnol. 23(3):337-43), or antisense oligonucleotides that recognize an ARRS. RNAi, ribozymes, and antisense oligonucleotides that recognize an ARRS can be designed and constructed using any method known to those of skill in the art. This system is particularly desirable if the therapeutic transgene is administered to treat cancer or to mediate host immune response.

In one embodiment, expression of the ablator must be controlled by an inducible promoter that provides tight control over the transcription of the ablator gene e.g., a pharmacological agent, or transcription factors activated by a pharmacological agent or in alternative embodiments, physiological cues. Promoter systems that are non-leaky and that can be tightly controlled are preferred. Inducible promoters suitable for controlling expression of the ablator are e.g., response elements including but not limited to a tetracycline (tet) response element (such as described by Gossen & Bujard (1992, Proc. Natl. Acad. Sci. USA 89:5547-551); an ecdysone-inducible response element (No D et al., 1996, Proc. Natl. Acad. Sci. USA. 93:3346-3351) a metal-ion response element such as described by Mayo et al. (1982, Cell. 29:99-108); Brinster et al. (1982, Nature 296:39-42) and Searle et al. (1985, Mol. Cell. Biol. 5: 1480-1489); a heat shock response element such as described by Nouer et al. (in: Heat Shock Response, ed. Nouer, L., CRC, Boca Raton, Fla., pp 167-220, 1991); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the ablator can be controlled, for example, by the Tet-on/off system (Gossen et al., 1995, Science 268:1766-9; Gossen et al., 1992, Proc. Natl. Acad. Set. USA., 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA., 91(17):8180-4; Schillinger et al., 2005, Proc. Natl. Acad. Sci. USA. 102(39):13789-94); the humanized tamoxifen-dep regulatable system (Roscilli et al., 2002, Mol. Ther. 6(5):653-63); and the ecdysone-dep regulatable system (Rheoswitch; Karns et al., 2001, BMC Biotechnol. 1: 11; Palli et al., 2003, Eur J Biochem. 270(6):1308-15) to name but a few.

A chimeric enzyme may be controlled by a constitutive or an inducible promoter. In one embodiment, the system utilizes a chimeric endonuclease, wherein the nuclease has at least two domains, i.e., a catalytic domain and a sequence specific DNA binding domain, each of which are expressed under separately controlled promoters and which are operatively linked. When the two domains are expressed at the same time, the products of the two domains form a chimeric endonuclease. Typically, separate transcription units containing each of domains linked to a DNA binding domain are provided. Such DNA binding domains include, for example, zinc finger motifs, homeo domain motifs, HMG-box domains, STAT proteins, B3, helix-loop-helix, winged helix-turn-helix, leucine zipper, helix-turn-helix, winged helix, POU domains, DNA binding domains of repressors, DNA binding domains of oncogenes and naturally occurring sequence-specific DNA binding proteins that recognize >6 base pairs. [U.S. Pat. No. 5,436,150, issued Jul. 25, 1995].

In one embodiment, the expression of the ablator is under the control of an inducible promoter that is regulated by the dimerizable transcription factor domains described in Section 5.1.3. An example of such an inducible promoter includes, but is not limited to a GAL4 binding site minimum promoter, which is responsive to a GAL4 transcription factor. A GAL4 DNA binding domain or transactivation domain can also be fused to a steroid receptor, such as the ecdysone receptor (EcR). Still other suitable inducible promoters, such as are described herein, may be selected.

5.1.3. Dimerizable Transcription Factor Domain Unit

In one embodiment, the PITA system is designed such that the viral genome(s) of the replication-defective viruses are further engineered to contain a dimerizable units which are heterodimer fusion proteins. These units may be a dimerizable TF unit as defined herein or another dimerizable fusion protein unit (e.g., part of a chimeric enzyme). In such an instance, a dimerizer is used (see Section 5.1.4), which binds to the dimerizer binding domains and dimerizes (reversibly cross-links) the DNA binding domain fusion protein and the activation domain fusion protein, forming a bifunctional transcription factor. See, e.g., the Ariad ARGENT″ system, which is described in U.S. Publication No. 2002/0173474, U.S. Publication No. 200910100535, U.S. Pat. No. 5,834,266, U.S. Pat. No. 7,109,317, U.S. Pat. No. 7,485,441, U.S. Pat. No. 5,830,462, U.S. Pat. No. 5,869,337, U.S. Pat. No. 5,871,753, U.S. Pat. No. 6,011,018, U.S. Pat. No. 6,043,082, U.S. Pat. No. 6,046,047, U.S. Pat. No. 6,063,625, U.S. Pat. No. 6,140,120, U.S. Pat. No. 6,165,787, U.S. Pat. No. 6,972,193, U.S. Pat. No. 6,326,166, U.S. Pat. No. 7,008,780, U.S. Pat. No. 6,133,456, U.S. Pat. No. 6,150,527, U.S. Pat. No. 6,506,379, U.S. Pat. No. 6,258,823, U.S. Pat. No. 6,693,189, U.S. Pat. No. 6,127,521, U.S. Pat. No. 6,150,137, U.S. Pat. No. 6,464,974, U.S. Pat. No. 6,509,152, U.S. Pat. No. 6,015,709, U.S. Pat. No. 6,117,680, U.S. Pat. No. 6,479,653, U.S. Pat. No. 6,187,757, U.S. Pat. No. 6,649,595, U.S. Pat. No. 6,984,635, U.S. Pat. No. 7,067,526, U.S. Pat. No. 7,196,192, U.S. Pat. No. 6,476,200, U.S. Pat. No. 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety.

In one embodiment, by delivering a dimerizable unit, target cells are modified to co-express two fusion proteins that are dimerized by the pharmacologic agent used: one containing a DNA-binding domain (DBD) of the transcription factor that binds the inducible promoter controlling the ablator and the other containing a transcriptional activation domain (AD) of the transcription factor that activates the inducible promoter controlling the ablator, each fused to dimerizer binding domains. Expression of the two fusion proteins may be constitutive, or as an added safety feature, inducible. Where an inducible promoter is selected for expression of one of the fusion proteins, the promoter may regulatable, but different from any other inducible or regulatable promoters in the viral composition. Addition of a pharmacological agent, or “dimerizer” (described in Section 5.1.4) that can simultaneously interact with the dimerizer binding domains present in both fusion proteins results in recruitment of the AD fusion protein to the regulated promoter, initiating transcription of the ablator. By using dimerizer binding domains that have no affinity for one another in the absence of ligand and an appropriate minimal promoter, transcription is made absolutely dependent on the addition of the dimerizer. Suitably, a replication-defective virus composition of the invention may contain more than one dimerizable domain. The various replication-defective viruses in a composition may be of different stock, which provide different transcription units (e.g., a fusion protein to form a dimerable unit in situ) and/or additional ablators.

Fusion proteins containing one or more transcription factor domains are disclosed in WO 94/18317, PCT/US94/08008, Spencer et al, supra and Blau et al. (PNAS 1997 94:3076) which are incorporated by reference herein in their entireties. The design and use of such fusion proteins for ligand-mediated gene-knock out and for ligand-mediated blockade of gene expression or inhibition of gene product function are disclosed in PCT/US95/10591. Novel DNA binding domains and DNA sequences to which they bind which are useful in embodiments involving regulated transcription of a target gene are disclosed, e.g., in Pomeranz et al, 1995, Science 267:93 96. Those references provide substantial information, guidance and examples relating to the design, construction and use of DNA constructs encoding analogous fusion proteins, target gene constructs, and other aspects which may also be useful to the practitioner of the subject invention.

Preferably the DNA binding domain, and a fusion protein containing it, binds to its recognized DNA sequence with sufficient selectivity so that binding to the selected DNA sequence can be detected (directly or indirectly as measured in vitro) despite the presence of other, often numerous other, DNA sequences. Preferably, binding of the fusion protein comprising the DNA-binding domain to the selected DNA sequence is at least two, more preferably three and even more preferably more than four orders of magnitude greater than binding to anyone alternative DNA sequence, as measured by binding studies in vitro or by measuring relative rates or levels of transcription of genes associated with the selected DNA sequence as compared to any alternative DNA sequences. The dimerizable transcription factor (TF) domain units of the invention can encode DNA binding domains and activation domains of any transcription factor known in the art. Examples of such transcription factors include but are not limited to GAL4, ZFHD1, VP16, and NF-KB (p65).

The dimerizer binding domain encoded by a dimerizable unit of the invention can be any dimerizer binding domain described in U.S. Publication No. 2002/0173474, U.S. Publication No. 200910100535, U.S. Pat. No. 5,834,266, U.S. Pat. No. 7,109,317, U.S. Pat. No. 7,485,441, U.S. Pat. No. 5,830,462, U.S. Pat. No. 5,869,337, U.S. Pat. No. 5,871,753, U.S. Pat. No. 6,011,018, U.S. Pat. No. 6,043,082, U.S. Pat. No. 6,046,047, U.S. Pat. No. 6,063,625, U.S. Pat. No. 6,140,120, U.S. Pat. No. 6,165,787, U.S. Pat. No. 6,972,193, U.S. Pat. No. 6,326,166, U.S. Pat. No. 7,008,780, U.S. Pat. No. 6,133,456, U.S. Pat. No. 6,150,527, U.S. Pat. No. 6,506,379, U.S. Pat. No. 6,258,823, U.S. Pat. No. 6,693,189, U.S. Pat. No. 6,127,521, U.S. Pat. No. 6,150,137, U.S. Pat. No. 6,464,974, U.S. Pat. No. 6,509,152, U.S. Pat. No. 6,015,709, U.S. Pat. No. 6,117,680, U.S. Pat. No. 6,479,653, U.S. Pat. No. 6,187,757, U.S. Pat. No. 6,649,595, U.S. Pat. No. 6,984,635, U.S. Pat. No. 7,067,526, U.S. Pat. No. 7,196,192, U.S. Pat. No. 6,476,200, U.S. Pat. No. 6,492,106, WO 94118347, WO 96/20951, WO 96/06097, WO 97/31898, WO 96/41865, WO 98/02441, WO 95/33052, WO 99/10508, WO 99110510, WO 99/36553, WO 99/41258, WO 01114387, ARGENT™ Regulated Transcription Retrovirus Kit, Version 2.0 (Sep. 9, 2002), and ARGENT™ Regulated Transcription Plasmid Kit, Version 2.0 (Sep. 9, 2002), each of which is incorporated herein by reference in its entirety.

A dimerizer binding domain that can be used in the PITA system is the immunophilin FKBP (FK506-binding protein). FKBP is an abundant 12 kDa cytoplasmic protein that acts as the intracellular receptor for the immunosuppressive drugs FK506 and rapamycin. Regulated transcription can be achieved by fusing multiple copies of FKBP to a DNA binding domain of a transcription factor and an activation domain of a transcription factor, followed by the addition of FK1012 (a homodimer of FK506; Ho, S. N., et al., 1996, Nature, 382(6594): 822-6); or simpler synthetic analogs such as AP1510 (Amara, J. F., et al., 1997, Proc. Natl. Acad. Sci. USA, 94(20): 10618-23). The potency of these systems can be improved by using synthetic dimerizers, such as AP1889, with designed ‘bumps’ that minimize interactions with endogenous FKBP (Pollock et al., 1999, Methods Enzymol, 1999.306: p. 263-81). Improved approaches based on heterodimerization, exploiting the discovery that FK506 and rapamycin naturally function by bringing together FKBP with a second target protein. This allows the natural products themselves, or analogs thereof, to be used directly as dimerizers to control gene expression.

The structure of FKBP-FK506 complexed to calcineurin phosphatase (Griffith et al., Cell, 82:507 522, 1995) has been reported. Calcineurin A (residues 12 394) was shown to be effective as a dimerizer binding domain using a three hybrid system in yeast using three FKBPs fused to Gal4 and residues 12 to 394 of murine calcineurin A fused C-terminally to the Gal4 activation domain (Ho, 1996 Nature. 382:822 826). Addition of FK506 activated transcription of a reporter gene in these cells. A “minimal” calcineurin domain termed a CAB, which is a smaller, more manipulatable domain can be used as a dimerizer binding domain.

The DNA binding domain fusion protein and activation domain fusion protein encoded by the dimerizable fusion protein units of the invention may contain one or more copies of one or more different dimerizer binding domains. The dimerizer binding domains may be N-terminal, C-terminal, or interspersed with respect to the DNA binding domain and activation domain. Embodiments involving multiple copies of a dimerizer binding domains usually have 2, 3 or 4 such copies. The various domains of the fusion proteins are optionally separated by linking peptide regions which may be derived from one of the adjacent domains or may be heterologous.

As used herein, the term “variants” in the context of variants of dimerizer binding domains refers to dimerizer binding domains that contain deletions, insertions, substitutions, or other modifications relative to native dimerizer binding domains, but that retain their specificity to bind to dimerizers. The variants of dimerizer binding domains preferably have deletions, insertions, substitutions, and/or other modifications of not more than 10, 9, 8, 7, 6, 5, 4, 3, 2, or 1 amino acid residues. In a specific embodiment, the variant of a dimerizer binding domain has the native sequence of a dimerizer binding domain as specified above, except that 1 to 5 amino acids are added or deleted from the carboxy and or the amino end of the dimerizer binding domains (where the added amino acids are the flanking amino acid(s) present in the native dimerizer binding domains).

In order to conserve space within the viral genome(s), bicistronic transcription units can be engineered. For example, the third and fourth transcription units can be engineered as a bicistronic unit containing an IRES (internal ribosome entry site), which allows coexpression of heterologous gene products by a message from a single promoter. Alternatively, a single promoter may direct expression of an RNA that contains, in a single open reading frame (ORF), two or three heterologous genes (e.g., the third and fourth transcription units) separated from one another by sequences encoding a self-cleavage peptide (e.g., T2A) or a protease recognition site (e.g., furin). The ORF thus encodes a single polyprotein, which, either during (in the case of T2A) or after translation, is cleaved into the individual proteins. It should be noted, however, that although these IRES and polyprotein systems can be used to save AAV packaging space, they can only be used for expression of components that can be driven by the same promoter.

As illustrated in the examples below, various components of the invention may include:

ITR: inverted terminal repeats (ITR) of AAV serotype 2 (168 bp). In one embodiment, the AAV2 ITRs are selected to generate a pseudotyped AAV, i.e., an AAV having a capsid from a different AAV than that the AAV from which the ITRs are derived.

CMV: full cytomegalovirus (CMV) promoter; including enhancer. CMV: minimal CMV promoter, not including enhancer. In one embodiment, the human CMV promoter and/or enhancer are selected.

FRB-TA fusion: fusion of dimerizer binding domain and an activation domain of a transcription factor. The FRB fragment corresponds to amino acids 2021-2113 of FRAP (FKBP rapamycin-associated protein, also known as mTOR [mammalian target of rapamycin]), a phosphoinositide 3-kinase homolog that controls cell growth and division. The FRAP sequence incorporates the single point-mutation Thr2098Leu (FRAP_{L}) to allow use of certain non-immunosuppressive rapamycin analogs (rapalogs). FRAP binds to rapamycin (or its analogs) and FKBP and is fused to a portion of human NF-KB p65 (190 amino acids) as transcription activator.

ZFHD-FKBP fusion: fusion of a DNA binding domain and 1 copy of a Dimerizer binding domain, 2 copies of drug binding domain (2xFKBP, or 3 (3xFKBP) copies of drug binding domain. Immunophilin FKBP (FK506-binding protein) is an abundant 12 kDa cytoplasmic protein that acts as the intracellular receptor for the immunosuppressive drugs FK506 and rapamycin. ZFHD is DNA binding domains composed of a zinc finger pair and a homeodomain. In another alternative, various other copy numbers of a selected drug binding domain may be selected. Such fusion proteins may contain N-terminal nuclear localization sequence from human c-Myc at the 5′ and/or 3′ end.

Z8I: contains 8 copies of the binding site for ZFHD (Z8) followed by minimal promoter from the human interleukin-2 (IL-2) gene (SEQ ID NO: 32). Variants of this may be used, e.g., which contain from 1 to about 20 copies of the binding site for ZFHD followed by a promoter, e.g., the minimal promoter from IL-2 or another selected promoter.

Cre: Cre recombinase. Cre is a type I topoisomerase isolated from bacteriophage P1. Cre mediates site specific recombination in DNA between two loxP sites leading to deletion or gene conversion (1029 bp, SEQ ID NO: 33).

I-SceI: a member of intron endonuclease or homing endonuclease which is a large class of meganuclease (708 bp, SEQ ID NO: 34). They are encoded by mobile genetic elements such as introns found in bacteria and plants. I-SceI is a yeast endonuclease involved in an intron homing process. I-SceI recognizes a specific asymmetric 18 bp element, a rare sequence in mammalian genome, and creates double strand breaks. See, Jasin, M. (1996) Trends Genet., 12, 224-228.

hGH poly A: minimal poly adenylation signal from human GH (SEQ ID NO: 35).

IRES: internal ribosome entry site sequence from ECMV (encephalomyocarditis virus) (SEQ ID NO: 36).

5.1.4. Dimerizers and Pharmacologic Agents

As used herein, the term “dimerizer” is a compound that can bind to dimerizer binding domains of the TF domain fusion proteins (described in Section 5.1.3) and induce dimerization of the fusion proteins. Any pharmacological agent that dimerizes the domains of the transcription factor, as assayed in vitro can be used. Preferably, rapamycin and its analogs referred to as “rapalogs” can be used. Any of the dimerizers described in following can be used: U.S. Publication No. 2002/0173474, U.S. Publication No. 2009/0100535, U.S. Pat. No. 5,834,266, U.S. Pat. No. 7,109,317, U.S. Pat. No. 7,485,441, U.S. Pat. No. 5,830,462, U.S. Pat. No. 5,869,337, U.S. Pat. No. 5,871,753, U.S. Pat. No. 6,011,018, U.S. Pat. No. 6,043,082, U.S. Pat. No. 6,046,047, U.S. Pat. No. 6,063,625, U.S. Pat. No. 6,140,120, U.S. Pat. No. 6,165,787, U.S. Pat. No. 6,972,193, U.S. Pat. No. 6,326,166, U.S. Pat. No. 7,008,780, U.S. Pat. No. 6,133,456, U.S. Pat. No. 6,150,527, U.S. Pat. No. 6,506,379, U.S. Pat. No. 6,258,823, U.S. Pat. No. 6,693,189, U.S. Pat. No. 6,127,521, U.S. Pat. No. 6,150,137, U.S. Pat. No. 6,464,974, U.S. Pat. No. 6,509,152, U.S. Pat. No. 6,015,709, U.S. Pat. No. 6,117,680, U.S. Pat. No. 6,479,653, U.S. Pat. No. 6,187,757, U.S. Pat. No. 6,649,595, U.S. Pat. No. 6,984,635, U.S. Pat. No. 7,067,526, U.S. Pat. No. 7,196,192, U.S. Pat. No. 6,476,200, U.S. Pat. No. 6,492,106, WO 94118347, WO 96/20951, WO 96/06097, WO 97/31898, WO 96/41865, WO 98/02441, WO 95/33052, WO 99/10508, WO 99/10510, WO 99/36553, WO 99/41258, WO 01114387, ARGENT™ Regulated Transcription Retrovirus Kit, Version 2.0 (9109/02), and ARGENT™ Regulated Transcription Plasmid Kit, Version 2.0 (Sep. 9, 2002), each of which is incorporated herein by reference in its entirety.

Examples of dimerizers that can be used in the present invention include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP.

Other dimerizers capable of binding to dimerizer binding domains or to other endogenous constituents may be readily identified using a variety of approaches, including phage display and other biological approaches for identifying peptidyl binding compounds; synthetic diversity or combinatorial approaches (see e.g. Gordon et al, 1994, J Med Chem 37(9):1233-1251 and 37(10):1385-1401); and DeWitt et al, 1993, PNAS USA 90:6909-6913) and conventional screening or synthetic programs. Dimerizers capable of binding to dimerizer binding domains of interest may be identified by various methods of affinity purification or by direct or competitive binding assays, including assays involving the binding of the protein to compounds immobilized on solid supports such as pins, beads, chips, etc.). See e.g. Gordon et al., supra.

Generally speaking, the dimerizer is capable of binding to two (or more) protein molecules, in either order or simultaneously, preferably with a Kd value below about 10⁻⁶ more preferably below about 10⁻⁷, even more preferably below about 10⁻⁸, and in some embodiments below about 10⁻⁹ M. The dimerizer preferably is a non-protein and has a molecular weight of less than about 5 kDa. The proteins so oligomerized may be the same or different.

Various dimerizers are hydrophobic or can be made so by appropriate modification with lipophilic groups. Particularly, dimerizers containing linking moieties can be modified to enhance lipophilicity by including one or more aliphatic side chains of from about 12 to 24 carbon atoms in the linker moiety.

5.1.5. Generating Replication-Defective Virus Compositions

Any virus suitable for gene transfer (e.g. gene therapy) may be used for packaging the transcription units into one or more stocks of replication-defective virus, including but not limited to adeno-associated virus (“AAV”); adenovirus; alphavirus; herpesvirus; retrovirus (e.g., lentivirus); vaccinia virus; etc. Methods well known in the art for packaging foreign genes into replication-defective viruses can be used to prepare the replication-defective viruses containing the therapeutic transgene unit, the ablation unit, and optionally (but preferably) the dimerizable transcription factor domain unit. See, for example, Gray & Samulski, 2008, “Optimizing gene delivery vectors for the treatment of heart disease,” Expert Opin. Biol. Ther. 8:911-922; Murphy & High, 2008, “Gene therapy for haemophilia,” Br. J. Haematology 140:479-487; Hu, 2008, “Baculoviral vectors for gene delivery: A review,” Current Gene Therapy 8:54-65; Gomez et al., 2008, “The poxvirus vectors MV A and NYV AC as gene delivery systems for vaccination against infectious diseases and cancer,” Current Gene Therapy 8:97-120.

In preferred embodiments, the replication-deficient virus compositions for therapeutic use are generated using an AAV. Methods for generating and isolating AAVs suitable for gene therapy are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety.

Adeno-associated virus (genus Dependovirus, family Parvoviridae) is a small (approximately 20-26 nm), non-enveloped single-stranded (ss) DNA virus that infects humans and other primates. Adeno-associated virus is not currently known to cause disease. Adeno-associated virus can infect both dividing and non-dividing cells. In the absence of functional helper virus (for example, adenovirus or herpesvirus) AAV is replication-defective. Adeno-associated viruses form episomal concatamers in the host cell nucleus. In non-dividing cells, these concatamers remain intact for the life of the host cell. In dividing cells, AAV DNA is lost through cell division, since the episomal DNA is not replicated along with the host cell DNA. However, AAV DNA may also integrate at low levels into the host genome.

The AAV genome is built of a ssDNA, either positive- or negative-sense, which is about 4.7 kilobases long. The genome of AAV as it occurs in nature comprises inverted terminal repeats (ITRs) at both ends of the DNA strand, and two open reading frames (ORFs): rep and cap. The former is composed of four overlapping genes encoding the Rep proteins that are required for the AAV life cycle, and the latter contains overlapping sequences that encode the capsid proteins (Cap): VP1, VP2, and VP3, which interact to form a capsid of an icosahedral symmetry.

The ITRs are 145 bases each, and form a hairpin that contributes to so-called “self-priming” that allows primase-independent synthesis of the second DNA strand. The ITRs also appear to be required for AAV DNA integration into the host cell genome (e.g., into the 19th chromosome in humans) and rescue from it, as well as for efficient encapsidation of the AAV DNA and assembly of AAV particles.

For packaging a transgene into virions, the ITRs are the only AAV components required in cis in the same construct as the transgene. The cap and rep genes can be supplied in trans. Accordingly, DNA constructs can be designed so that the AAV ITRs flank one or more of the transcription units (i.e., the transgene unit, the ablator unit, and the dimerizable transcription factor unit), thus defining the region to be amplified and packaged—the only design constraint being the upper limit of the size of the DNA to be packaged (approximately 4.5 kb). Adeno-associated virus engineering and design choices that can be used to save space are described below.

### Methods for Generating the Replication-Defective Virus Compositions

Many methods have been established for the efficient production of recombinant AAVs (rAAVs) that package a transgene—these can be used or adapted to generate the replication-defective virus compositions of the invention. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In a third system, a stable cell line that supplies the transgene flanked by ITRs and rep/cap is used. One method for minimizing the possibility of generating replication competent AAV (rcAAV) using these systems is by eliminating regions of homology between regions flanking the rep/cap cassette and the ITRs that flank the transgene. However, in each of these systems, AAV virions are produced in response to infection with helper adenovirus or herpes-virus, requiring the separation of the rAAVs from contaminating virus.

More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Grieger & Samulski, 2005; and Btining et al., 2008; Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See also the paragraphs below, which describe methods for scaling up AAV production using these systems and variants thereof.

Due to size constraints of AAV for packaging (tolerating a transgene of approximately 4.5 kb), the transcription unites) (i.e., the transgene unit, the ablator unit, and the dimerizable transcription factor unit) described may need to be engineered and packaged into two or more replication-deficient AAV stocks. This may be preferable, because there is evidence that exceeding the packaging capacity may lead to the generation of a greater number of “empty” AAV particles.

Alternatively, the available space for packaging may be conserved by combining more than one transcription unit into a single construct, thus reducing the amount of required regulatory sequence space. For example, a single promoter may direct expression of a single RNA that encodes two or three or more genes of interest, and translation of the downstream genes are driven by IRES sequences. In another example, a single promoter may direct expression of an RNA that contains, in a single open reading frame (ORF), two or three or more genes of interest separated from one another by sequences encoding a self-cleavage peptide (e.g., T2A) or a protease recognition site (e.g., furin). The ORF thus encodes a single polyprotein, which, either during (in the case of T2A) or after translation, is cleaved into the individual proteins (such as, e.g., transgene and dimerizable transcription factor). It should be noted, however, that although these IRES and polyprotein systems can be used to save AAV packaging space, they can only be used for expression of components that can be driven by the same promoter.

In another alternative, the transgene capacity of AAV can be increased by providing AAV ITRs of two genomes that can anneal to form head to tail concatamers. Generally, upon entry of the AAV into the host cell, the single-stranded DNA containing the transgene is converted by host cell DNA polymerase complexes into double-stranded DNA, after which the ITRs aid in concatamer formation in the nucleus. As an alternative, the AAV may be engineered to be a self-complementary (sc) AAV, which enables the virus to bypass the step of second-strand synthesis upon entry into a target cell, providing an scAAV virus with faster and, potentially, higher (e.g., up to 100-fold) transgene expression. For example, the AAV may be engineered to have a genome comprising two connected single-stranded DNAs that encode, respectively, a transgene unit and its complement, which can snap together following delivery into a target cell, yielding a double-stranded DNA encoding the transgene unit of interest. Self-complementary AAVs are described in, e.g., U.S. Pat. Nos. 6,596,535; 7,125,717; and 7,456,683, each of which is incorporated herein by reference in its entirety.

The transcription units(s) in the replication-deficient rAAVs may be packaged with any AAV capsid protein (Cap) described herein, known in the art, or to be discovered. Caps from serotypes AAV1, AAV6, AAV7, AAV8, AAV9 or rh10 are particularly preferred for generating rAAVs for use in human subjects. In a preferred embodiment, an rAAV Cap is based on serotype AAV8. In another embodiment, an rAAV Cap is based on Caps from two or three or more AAV serotypes. For example, in one embodiment, an rAAV Cap is based on AAV6 and AAV9.

Cap proteins have been reported to have effects on host tropism, cell, tissue, or organ specificity, receptor usage, infection efficiency, and immunogenicity of AAV viruses. See, e.g., Grieger & Samulski, 2005; Buning et al., 2008; and the references cited below in this sub-section; all of which are incorporated herein by reference in their entirety. Accordingly, an AAV Cap for use in an rAAV may be selected based on consideration of, for example, the subject to be treated (e.g., human or non-human, the subject's immunological state, the subject's suitability for long or short-term treatment, etc.) or a particular therapeutic application (e.g., treatment of a particular disease or disorder, or delivery to particular cells, tissues, or organs).

In some embodiments, an rAAV Cap is selected for its ability to efficiently transduce a particular cell, tissue, or organ, for example, to which a particular therapy is targeted. In some embodiments, an rAAV Cap is selected for its ability to cross a tight endothelial cell barrier, for example, the blood-brain barrier, the blood-eye barrier, the blood-testes barrier, the blood-ovary barrier, the endothelial cell barrier surrounding the heart, or the blood-placenta barrier.

Tissue specificity of adeno-associated viruses (AAV) serotypes is determined by the serotype of the capsid, and viral vector based on different AAV capsids may generated taking into consideration their ability to infect different tissues. AAV2 presents a natural tropism towards skeletal muscles, neurons of the central nervous system, vascular smooth muscle cells. AAV1 has been described as being more efficient than AAV2 in transducing muscle, arthritic joints, pancreatic islets, heart, vascular endothelium, central nervous system (CNS) and liver cells, whereas AAV3 appears to be well suited for the transduction of cochlear inner hair cells, AAV4 for brain, AAV5 for CNS, lung, eye, arthritic joints and liver cells, AAV6 for muscle, heart and airway epithelium, AAV7 for muscle, AAV8 for muscle, pancreas, heart and liver, and AAV9 for heart. See, e.g., Buning et at., 2008. Any serotype of AAV known in the art, e.g., serotypes AAV1, AAV2, AAV3A, AAV3B, AAV4, AAV5, AAV6, AAV7 [see, WO 2003/042397], AAV8 [see, e.g., U.S. Pat. No. 7,790,449; U.S. Pat. No. 7,282,199], AAV9 [see, WO 2005/033321], AAV10, AAV11, AAV12, rh10, modified AAV [see, e.g., WO 2006/110689], or yet to be discovered, or a recombinant AAV based thereon, may be used as a source for the rAAV capsid.

Various naturally occurring and recombinant AAVs, their encoding nucleic acids, AAV Cap and Rep proteins and their sequences, as well as methods for isolating or generating, propagating, and purifying such AAVs, and in particular, their capsids, suitable for use in producing rAAVs are described in Gao et al., 2004, “Clades of adeno-associated viruses are widely disseminated in human tissues,” J. Virol. 78:6381-6388; U.S. Pat. Nos. 7,319,002; 7,056,502; 7,282,199; 7,198,951; 7,235,393; 6,156,303; and 7,220,577; U.S. Patent Application Publication Nos. US 2003-0138772; US 2004-0052764; US 2007-0036760; US 2008-0075737; and US 2008-0075740; and International Patent Application Publication Nos. WO 20031014367; WO 20011083692; WO 2003/042397 (AAV7 and various simian AAV); WO 2003/052052; WO 2005/033321; WO 20061110689; WO 2008/027084; and WO 2007/127264; each of which is incorporated herein by reference in its entirety.

In some embodiments, an AAV Cap for use in the rAAV can be generated by mutagenesis (i.e., by insertions, deletions, or substitutions) of one of the aforementioned AAV Caps or its encoding nucleic acid. In some embodiments, the AAV Cap is at least 70% identical, 75% identical, 80% identical, 85% identical, 90% identical, 95% identical, 98% identical, or 99% or more identical to one or more of the aforementioned AAV Caps.

In some embodiments, the AAV Cap is chimeric, comprising domains from two or three or four or more of the aforementioned AAV Caps. In some embodiments, the AAV Cap is a mosaic of Vp1, Vp2, and Vp3 monomers from two or three different AAVs or recombinant AAVs. In some embodiments, an rAAV composition comprises more than one of the aforementioned Caps.

In some embodiments, an AAV Cap for use in an rAAV composition is engineered to contain a heterologous sequence or other modification. For example, a peptide or protein sequence that confers selective targeting or immune evasion may be engineered into a Cap protein. Alternatively or in addition, the Cap may be chemically modified so that the surface of the rAAV is polyethylene glycolated (PEGylated), which may facilitate immune evasion. The Cap protein may also be mutagenized, e.g., to remove its natural receptor binding, or to mask an immunogenic epitope.

### Methods for Scalable Manufacture of AAV

Methods for the scalable (e.g., for production at commercial scale) manufacture of AAV, which may be adapted in order to generate rAAV compositions that are suitably homogeneous and free of contaminants for use in clinical applications, are also known in the art, and are summarized briefly below.

Adeno-associated viruses can be manufactured at scale using a mammalian cell line-based approach, such as the approach using stable producer cell lines described in Thome et al., 2009, “Manufacturing recombinant adeno-associated viral vectors from producer cell clones,” Human Gene Therapy 20:707-714, which is incorporated herein by reference in its entirety. In the approach described by Thorpe and colleagues, producer cell lines stably containing all the components needed to generate an rAAV—the transgene construct (transgene flanked by ITRs) and AAV rep and cap genes—are engineered, which are induced to make virus by infection with a helper virus, such as a live adenovirus type 5 (Ad5) (methods of scalable production of which are also well known in the art). Producer cell lines are stably transfected with construct(s) containing (i) a packaging cassette (rep and cap genes of the desired serotype and regulatory elements required for their expression), (ii) the transgene flanked by ITRs, (iii) a selection marker for mammalian cells, and (iv) components necessary for plasmid propagation in bacteria. Stable producer cell lines are obtained by transfecting the packaging construct(s), selecting drug-resistant cells, and replica-plating to ensure production of the recombinant AAV in the presence of helper virus, which are then screened for performance and quality. Once appropriate clones are chosen, growth of the cell lines is scaled up, the cells are infected with the adenovirus helper, and resulting rAAVs are harvested from the cells.

In an alternative to the methods described in Thorpe et al., a packaging cell line is stably transfected with the AAV rep and cap genes., and the transgene construct is introduced separately when production of the rAAV is desired. Although Thorpe and colleagues use HeLa cells for the producer cell line, any cell line (e.g., Vera, A549, HEK 293) that is susceptible to infection with helper virus, able to maintain stably integrated copies of the rep gene and, preferably, able to grow well in suspension for expansion and production in a bioreactor may be used in accordance with the methods described in Thorpe et al.

In the foregoing methods, rAAVs are produced using adenovirus as a helper virus. In a modification of these methods, rAAVs can be generated using producer cells stably transfected with one or more constructs containing adenovirus helper functions, avoiding the requirement to infect the cells with adenovirus. In a variation, one or more of the adenovirus helper functions are contained within the same construct as the rep and cap genes. In these methods, expression of the adenovirus helper functions may be placed under transcriptional or post-transcriptional control to avoid adenovirus-associated cytotoxicity.

In an alternative to producing stable cell lines, AAVs may also be produced at scale using transient transfection methods, such as described by Wright, 2009, “Transient transfection methods for clinical adeno-associated viral vector production,” Human Gene Therapy 20:698-706, which is incorporated herein by reference in its entirety. Wright's approach involves transfection of cells with constructs that contain (i) the transgene of interest flanked by ITRs; (ii) the AAV rep and cap genes; and (iii) helper virus (e.g., adenovirus) genes required to support genome replication and packaging (or alternatively, a helper virus, as described in Thorpe et al.), Alternatively, the adenovirus helper functions may be contained within the same construct as the rep and cap genes. Thus, rAAVs are produced without having to ensure stable transfection of the transgene and rep/cap constructs. This provides a flexible and quick method for generating AAVs, and is thus ideal for pre-clinical and early-phase clinical development. Recombinant AAVs can be generated by transiently transfecting mammalian cell lines with the constructs using transient transfection methods known in the art. For example, transfection methods most suited for large-scale production include DNA co-precipitation with calcium phosphate, the use of poly-cations such as polyethylenimine (PE), and cationic lipids.

The effectiveness of adenovirus as a helper has also been exploited to develop alternative methods for large-scale recombinant AAV production, for example using hybrid viruses based on adenovirus and AAV (an “Ad-AAV hybrid”). This production method has the advantage that it does not require transfection—all that is required for rAAV production is infection of the rep/cap packaging cells by adenoviruses. In this process, a stable rep/cap cell line is infected with a helper adenovirus possessing functional E 1 genes and, subsequently, a recombinant Ad-AAV hybrid virus in which the AAV transgene plus ITRs sequence is inserted into the adenovirus E1 region. Methods for generating Ad-AAV hybrids and their use in recombinant AAV production are described in Zhang et al., 2009, which is incorporated by reference herein in its entirety.

In another variation, rAAVs can be generated using hybrid viruses based on AAV and herpes simplex virus type 1 (HSV) (an “HSV/AAV hybrid”), such as described in Clement et al., 2009, “Large-scale adeno-associated viral vector production using a herpesvirus-based system enables manufacturing for clinical studies,” Human Gene Therapy 20:796-806, which is incorporated herein by reference in its entirety. This method expands on the possibility of using HSV as a helper virus for AAV production (well known in the art, and also reviewed in Clement et al.). Briefly, HSV/AAV hybrids comprise an AAV transgene construct within an HSV backbone. These hybrids can be used to infect producer cells that supply the rep/cap and herpesvirus helper functions, or can be used in co-infections with recombinant HSVs that supply the helper functions, resulting in generation of rAAVs encapsidating the transgene of interest.

In another method, rAAV compositions may produced at scale using recombinant baculovirus-mediated expression of AAV components in insect cells, for example, as described in Virag et al., 2009, “Producing recombinant adeno-associated virus in foster cells: Overcoming production limitations using a baculovirus-insect cell expression strategy,” Human Gene Therapy 20:807-817, which is incorporated herein by reference in its entirety. In this system, the well-known baculovirus expression vector (BEV) system is adapted to produce recombinant AAVs. For example, the system described by Virag et al. comprises the infection of Sf9 insect cells with two (or three) different BEVs that provide (i) AAV rep and cap (either in one or two BEVs) and (ii) the transgene construct. Alternatively, the Sf9 cells can be stably engineered to express rep and cap, allowing production of recombinant AAVs following infection with only a single BEV containing the transgene construct. In order to ensure stoichiometric production of the Rep and Cap proteins, the latter of which is required for efficient packaging, the BEVs can be engineered to include features that enable pre- and post-transcriptional regulation of gene expression. The Sf9 cells then package the transgene construct into AAV capsids, and the resulting rAAV can be harvested from the culture supernatant or by lysing the cells.

Each of the foregoing methods permit the scalable production of rAAV compositions. The manufacturing process for an rAAV composition suitable for commercial use (including use in the clinic) must also comprise steps for removal of contaminating cells; removing and inactivating helper virus (and any other contaminating virus, such as endogenous retrovirus-like particles); removing and inactivating any rcAAV; minimizing production of, quantitating, and removing empty (transgene-less) AAV particles (e.g., by centrifugation); purifying the rAAV (e.g., by filtration or chromatography based on size and/or affinity); and testing the rAAV composition for purity and safety. These methods are also provided in the references cited in the foregoing paragraphs and are incorporated herein for this purpose.

One disadvantage of the foregoing methods of scalable rAAV production is that much of the rAAV is obtained by lysing the producer cells, which requires significant effort to not only obtain the virus but also to isolate it from cellular contaminants. To minimize these requirements, scalable methods of rAAV production that do not entail cell lysis may be used, such as provided in International Patent Application Publication No. WO 2007/127264, the contents of which is incorporated by reference herein in its entirety. In the example of Section 6 infra, a new scalable method obtaining rAAV from cell culture supernatants is provided, which may also be adapted for the preparation of rAAV composition for use in accordance with the methods described herein.

In still another embodiment, the invention provides human or non-human cells which contain one or more of the DNA constructs and/or virus compositions of the invention. Such cells may be genetically engineered and may include, e.g., plant, bacterial, non-human mammalian or mammalian cells. Selection of the cell types is not a limitation of the invention.

### 5.2. Compositions

The present invention provides replication-defective virus compositions suitable for use in therapy (in vivo or ex vivo) in which the genome of the virus (or the collective genomes of two or more replication-defective virus stocks used in combination) comprise the therapeutic transgene unit and the ablator unit defined in Section 3.1, and described supra; and may further comprise dimerizable fusion protein or TF domain units(s) (referred to for purposes of convenience as dimerizable unit(s)). Any virus suitable for gene therapy may be used in the compositions of the invention, including but not limited to adeno-associated virus (“AAV”), adenovirus, herpes simplex virus, lentivirus, or a retrovirus. In a preferred embodiment, the compositions are replication-defective AAVs, which are described in more detail in Section 5.2.1 herein.

The compositions of the invention comprise a replication-defective virus(es) suitable for therapy (in vivo or ex vivo) in which the genome of the virus(es) comprises a transgene unit, an ablation unit, and/or a dimerizable unit. In one embodiment, a composition of the invention comprises a virus suitable for gene therapy in which the genome of the virus comprises a transgene unit. In another embodiment, a composition of the invention comprises a virus suitable for gene therapy in which the genome of the virus comprises an ablation unit. In another embodiment, a composition of the invention comprises a virus suitable for gene therapy in which the genome of the virus comprises a dimerizable unit. In another embodiment, a composition of the invention comprises a virus suitable for gene therapy in which the genome of the virus comprises a transgene unit and an ablation unit. In another embodiment, a composition of the invention comprises a virus suitable for gene therapy in which the genome of the virus comprises a transgene unit and a dimerizable unit. In another embodiment, a composition of the invention comprises a virus suitable for gene therapy in which the genome of the virus comprises an ablation unit and a dimerizable unit. In another embodiment, a composition of the invention comprises viruses suitable for gene therapy in which the genome of the virus comprises a transgene unit, an ablation unit and a dimerizable unit.

The invention also provides compositions comprising recombinant DNA constructs that comprise one or more transcriptional units described herein. Compositions comprising recombinant DNA constructs are described in more detail in Section 5.2.2.

5.2.1. Replication-Defective Virus Compositions for Gene Therapy

The invention provides compositions comprising a replication-defective virus stock(s) and formulations of the replication-defective virus(es) in a physiologically acceptable carrier. These formulations can be used for gene transfer and/or gene therapy. The viral genome of the compositions comprises: (a) a first transcription unit that encodes a therapeutic product in operative association with a promoter that controls transcription, said unit containing at least one ablation recognition site (transgene unit); and (b) a second transcription unit that encodes an ablator specific for the ablation recognition site, or a fragment thereof, in operative association with a promoter. In one embodiment, the viral genome of the replication-defective virus. The ablator is as defined elsewhere in this specification.

### AAV Stocks

In a preferred embodiment, the replication-defective virus of a composition of the invention is an AAV, preferably AAV1, AAV6, AAV6.2, AAV7, AAV8, AAV9 or rh10. In one embodiment, the AAV of the composition is AAV8. Due to the packaging constraints of AAV (approximately 4.5 kb) in most cases, for ease of manufacture, the transgene unit, the ablation unit, and the dimerizable unit will be divided between two or more viral vectors and packaged in a separate AAV stock. In one embodiment, the replication-defective virus composition comprises the first transcription unit (a transgene unit) packaged in one AAV stock, and the second (an ablator unit), third and fourth transcription units (dimerizable TF domain unit) packaged in a second AAV stock. In another embodiment, the replication-defective virus composition comprises the second transcription unit (an ablator unit) packaged in one AAV stock, and the first (a transgene unit), third and fourth transcription units (dimerizable TF domain unit) packaged in a second AAV stock. In another embodiment, all four units can be packaged in one AAV stock, but this imposes limits on the size of the DNAs that can be packaged. For example, when using Cre as the ablator and FRB/FKB as the dimerizable TF domains (as shown in the examples, infra), in order to package all four units into one AAV stock, the size of the DNA encoding the therapeutic transgene should be less than about 900 base pairs in length; this would accommodate DNAs encoding cytokines, RNAi therapeutics, and the like.

Due to size constraints of the AAV genome for packaging, the transcription units can be engineered and packaged in two or more AAV stocks. Whether packaged in one viral stock which is used as a virus composition according to the invention, or in two or more viral stocks which form a virus composition of the invention, the viral genome used for treatment must collectively contain the first and second transcription units encoding the therapeutic transgene and the ablator; and may further comprise additional transcription units (e.g., the third and fourth transcription units encoding the dimerizable TF domains). For example, the first transcription unit can be packaged in one viral stock, and second, third and fourth transcription units packaged in a second viral stock. Alternatively, the second transcription unit can be packaged in one viral stock, and the first, third and fourth transcription units packaged in a second viral stock. While useful for AAV due to size contains in packaging the AAV genome, other viruses may be used to prepare a virus composition according to the invention. In another embodiment, the viral compositions of the invention, where they contain multiple viruses, may contain different replication-defective viruses (e.g., AAV and adenovirus).

In one embodiment, a virus composition according to the invention contains two or more different AAV (or another viral) stock, in such combinations as are described above. For example, a virus composition may contain a first viral stock comprising the therapeutic gene with ablator recognition sites and a first ablator and a second viral stock containing an additional ablator(s). Another viral composition may contain a first virus stock comprising a therapeutic gene and a fragment of an ablator and a second virus stock comprising another fragment of an ablator. Various other combinations of two or more viral stocks in a virus composition of the invention will be apparent from the description of the components of the present system.

### Viral Formulations

Compositions of the invention may be formulated for delivery to animals for veterinary purposes (e.g., livestock (cattle, pigs, etc), and other non-human mammalian subjects, as well as to human subjects. The replication-defective viruses can be formulated with a physiologically acceptable carrier for use in gene transfer and gene therapy applications. Because the viruses are replication-defective, the dosage of the formulation cannot be measured or calculated as a PFU (plaque forming unit). Instead, quantification of the genome copies (“GC”) may be used as the measure of the dose contained in the formulation.

Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal):

Also, the replication-defective virus compositions can be formulated in dosage units to contain an amount of replication-defective virus that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

The replication-defective viruses can be formulated in a conventional manner using one or more physiologically acceptable carriers or excipients. The replication-defective viruses may be formulated for parenteral administration by injection, e.g., by bolus injection or continuous infusion. Formulations for injection may be presented in unit dosage form, e.g., in ampoules or in multi-dose containers, with an added preservative. The replication-defective virus compositions may take such forms as suspensions, solutions or emulsions in oily or aqueous vehicles, and may contain formulatory agents such as suspending, stabilizing and/or dispersing agents. Liquid preparations of the replication-defective virus formulations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts. Alternatively, the compositions may be in powder form for constitution with a suitable vehicle, e.g., sterile pyrogen-free water, before use.

Also encompassed is the use of adjuvants in combination with or in admixture with the replication-defective viruses of the invention. Adjuvants contemplated include but are not limited to mineral salt adjuvants or mineral salt gel adjuvants, particulate adjuvants, microparticulate adjuvants, mucosal adjuvants, and immunostimulatory adjuvants. Adjuvants can be administered to a subject as a mixture with replication-defective viruses of the invention, or used in combination with the replication-defective viruses of the invention.

5.2.2. Recombinant DNA Construct Compositions for Production of Replication-Defective Viral Vectors Useful for Therapeutic Purposes

The invention provides recombinant DNA construct compositions comprising a transgene unit, an ablation unit, and/or one or two dimerizable domain units flanked by viral signals that define the region to be amplified and packaged into replication-defective viral particles. These DNA constructs can be used to generate the replication-defective virus compositions and stocks.

In one embodiment, the recombinant DNA construct comprises a transgene unit flanked by packaging signals of a viral genome. In another embodiment, a composition of the invention comprises a recombinant DNA construct comprising an ablation unit flanked by packaging signals of a viral genome. In another embodiment, the recombinant DNA construct comprises a dimerizable unit flanked by packaging signals of a viral genome. In another embodiment, the recombinant DNA construct comprises a transgene unit and an ablation unit flanked by packaging signals of a viral genome. In another embodiment, the recombinant DNA construct comprises a transgene unit and a dimerizable unit flanked by packaging signals of a viral genome. In another embodiment, the recombinant DNA construct comprises an ablation unit and a dimerizable unit flanked by packaging signals of a viral genome. In another embodiment, the recombinant DNA construct comprises a transgene unit, an ablation unit and a dimerizable unit flanked by packaging signals of a viral genome.

The first transcription unit encodes a therapeutic product in operative association with a promoter that controls transcription, said unit containing at least one ablation recognition site (transgene unit); and (b) the second transcription unit that encodes an ablator specific for the ablation recognition site, or a fragment thereof fused to a binding domain, in operative association with a promoter that induces transcription in response to a pharmacological agent (ablation unit). In another embodiment, the recombinant DNA construct comprises a dimerizable TF domain unit flanked by packaging signals of a viral genome.

In a preferred embodiment, the recombinant DNA construct composition further comprises a dimerizable unit nested within the viral packaging signals. In one embodiment, each unit encodes a dimerizable domain of a transcription factor that regulates the inducible promoter of the second transcription unit, in which (c) a third transcription unit encodes the DNA binding domain of the transcription factor fused to a binding domain for the pharmacological agent in operative association with a constitutive promoter; and (d) a fourth transcription unit encodes the activation domain of the transcription factor fused to a binding domain for the pharmacological agent in operative association with a constitutive promoter. In another embodiment, at least one of (c) or (d) is expressed under an inducible promoter. In a specific embodiment, the pharmacological agent that induces transcription of the promoter that is in operative association with the second unit of the recombinant DNA construct composition is a dimerizer that dimerizes the domains of the transcription factor as measured in vitro. In yet another specific embodiment, the pharmacological agent that induces transcription of the promoter that is in operative association with the second unit of the recombinant DNA construct composition is rapamycin. In still a further embodiment, the recombinant DNA construct comprises a dimerizable fusion protein unit. For example, the dimerizable fusion protein unit may be encode (a) a binding domain of an enzyme fused to a binding domain and (b) a catalytic domain of the enzyme fused to a binding domain, where the binding domains are either DNA binding domains or the binding domains for a dimerizer.

In order to conserve space within the viral genome(s), bicistronic transcription units can be engineered. For example, the third and fourth transcription units can be engineered as a bicistronic unit containing an IRES (internal ribosome entry site), which allows coexpression of heterologous gene products by a message from a single promoter. Alternatively, a single promoter may direct expression of an RNA that contains, in a single open reading frame (ORF), two or three heterologous genes (e.g., the third and fourth transcription units) separated from one another by sequences encoding a self-cleavage peptide (e.g., T2A) or a protease recognition site (e.g., furin). The ORF thus encodes a single polyprotein, which, either during (in the case of T2A) or after translation, is cleaved into the individual proteins. It should be noted, however, that although these IRES and polyprotein systems can be used to save AAV packaging space, they can only be used for expression of components that can be driven by the same promoter.

In a specific embodiment, a recombinant DNA construct composition that comprises a dimerizable unit comprises an IRES. In another specific embodiment, a recombinant DNA construct composition that comprises a third and fourth transcription unit (a dimerizable TF domain unit) comprises and IRES. In another specific embodiment, a recombinant DNA construct composition that comprises a transgene unit comprises an IRES. In another specific embodiment, a recombinant DNA construct composition that comprises an ablation unit comprises an IRES. In another specific embodiment, a recombinant DNA construct composition that comprises a dimerizable unit comprises an IRES.

In a specific embodiment, a recombinant DNA construct composition that comprises a third and a fourth transcription unit (a dimerizable TF domain unit) comprises T2A sequence. In another specific embodiment, a recombinant DNA construct composition that comprises a transgene unit comprises T2A sequence. In another specific embodiment, a recombinant DNA construct composition that comprises an ablation unit comprises T2A sequence. In another specific embodiment, a recombinant DNA construct composition that comprises a dimerizable TF domain unit comprises T2A sequence.

In an embodiment, the ablator that is encoded by the second transcription unit of the recombinant DNA construct composition is an endonuclease, a recombinase, a meganuclease, or an artificial zinc finger endonuclease that binds to the ablation recognition site in the first transcription unit and excises or ablates DNA. In a specific embodiment, the ablator is ere and the ablation recognition site is LoxP, or the ablator is FLP and the ablation recognition site is FRT. In another embodiment, the ablator that is encoded by the second transcription unit of the recombinant DNA construct composition is an interfering RNA, a ribozyme, or an antisense that ablates the RNA transcript of the first transcription unit, or suppresses translation of the RNA transcript of the first transcription unit. In a specific embodiment, transcription of the ablator is controlled by a tet-on/off system, a tetR-KRAB system, a mifepristone (RU486) regulatable system, a tamoxifen-dep regulatable system, or an ecdysone-dep regulatable system.

The recombinant DNA construct composition contains packaging signals flanking the transcription units desired to be amplified and packaged in replication-defective virus vectors. In a specific embodiment, the packaging signals are AAV ITRs. Where a pseudotyped AAV is to be produced, the ITRs are selected from a source which differs from the AAV source of the capsid. For example, AAV2 ITRs may be selected for use with an AAV1, AAV8, or AAV9 capsid, and so on. In another specific embodiment, the AAV ITRs may be from the same source as the capsid, e.g., AAV1, AAV6, AAV7, AAV8, AAV9, rh10 ITRs, etc. In another specific embodiment, a recombinant DNA construct composition comprises a first transcription unit (transgene unit) flanked by AAV ITRs, and the second (ablation unit), and optional third and fourth transcription units (a dimerizable TF domain unit), and/or a dimerizable fusion protein unit(s), flanked by AAV ITRs. In yet another specific embodiment, a recombinant DNA construct composition comprises a second transcription unit (ablation unit) flanked by AAV ITRs, and the first (transgene unit), third and fourth transcription units (a dimerizable TF domain unit) are flanked by AAV ITRs. In a preferred embodiment, the transcription units of a PIT A system are contained in two or more recombinant DNA compositions.

In a specific embodiment, recombinant DNA construct contains a transgene unit that encodes anyone or more of the following therapeutic products: an antibody or antibody fragment that neutralizes HIV infectivity, soluble vascular endothelial growth factor receptor-1 (sFlt-I), Factor VIII, Factor IX, insulin like growth factor (IGF), hepatocyte growth factor (HGF), heme oxygenase-1 (HO-1), or nerve growth factor (NGF). In a specific embodiment, recombinant DNA construct contains a transgene unit that comprises anyone of the following promoters that controls transcription of the therapeutic gene: a constitutive promoter, a tissue-specific promoter, a cell-specific promoter, an inducible promoter, or a promoter responsive to physiologic cues.

The DNA constructs can be used in any of the methods described in Section 5.1.5 to generate replication-defective virus stocks.

5.2.3. Pharmaceutical Compositions and Formulations of Dimerizers

The present invention provides pharmaceutical compositions comprising the dimerizers of the invention, described in Section 5.1.4. In a preferred embodiment, the pharmaceutical compositions comprise a pharmaceutically acceptable carrier or excipient. Optionally, these pharmaceutical compositions are adapted for veterinary purposes, e.g., for delivery to a non-human mammal (e.g., livestock), such as are described herein.

The pharmaceutical compositions of the invention can be administered to a subject at therapeutically effective doses to ablate or excise the transgene of a transgene unit of the invention or to ablate the transcript of the transgene, or inhibit its translation. A therapeutically effective dose refers to an amount of the pharmaceutical composition sufficient to result in amelioration of symptoms caused by expression of the transgene, e.g., toxicity, or to result in at least 60%, 65%, 70%, 75%, 80%, 85%, 90%, 95%, or 100% inhibition of expression of the transgene.

In an embodiment, an amount of pharmaceutical composition comprising a dimerizer of the invention is administered that is in the range of about 0.1-5 micrograms (μg)/kilogram (kg). To this end, a pharmaceutical composition comprising a dimerizer of the invention is formulated in doses in the range of about 7 mg to about 350 mg to treat to treat an average subject of 70 kg in body weight. The amount of pharmaceutical composition comprising a dimerizer of the invention administered is: 0.1, 0.2, 0.3, 0.4, 0.5, 0.6, 0.7, 0.8, 0.9, 1.0, 1.5, 2.0, 2.5, 3.0, 3.5, 4.0, 4.5 or 5.0 mg/kg. The dose of a dimerizer in a formulation is 7, 8, 9, 10, 20, 25, 30, 35, 40, 45, 50, 55, 60, 65, 70, 75, 80, 85 90, 95, 100, 125, 150, 175, 200, 225, 250, 275, 300, 325, 350, 375, 400, 400, 425, 450, 475, 500, 525, 550, 575, 600, 625, 650, 675, 700, 725, or 750 mg (to treat to treat an average subject of 70 kg in body weight). These doses are preferably administered orally. These doses can be given once or repeatedly, such as daily, every other day, weekly, biweekly, or monthly. Preferably, the pharmaceutical compositions are given once weekly for a period of about 4-6 weeks. In some embodiments, a pharmaceutical composition comprising a dimerizer is administered to a subject in one dose, or in two doses, or in three doses, or in four doses, or in five doses, or in six doses or more. The interval between dosages may be determined based the practitioner's determination that there is a need for inhibition of expression of the transgene, for example, in order to ameliorate symptoms caused by expression of the transgene, e.g., toxicity. For example, in some embodiments when the need for transgene ablation is acute, daily dosages of a pharmaceutical composition comprising a dimerizer may be administered. In other embodiments, e.g., when the need for transgene ablation is less acute, or is not acute, weekly dosages of a pharmaceutical composition comprising a dimerizer may be administered.

Pharmaceutical compositions for use in accordance with the present invention may be formulated in conventional manner using one or more physiologically acceptable carriers or excipients. Thus, the dimerizers and their physiologically acceptable salts and solvates may be formulated for administration by inhalation or insufflation (either through the mouth or the nose) oral, buccal, parenteral, rectal, or transdermal administration. Noninvasive methods of administration are also contemplated.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the dimerizers.

For buccal administration the compositions may take the form of tablets or lozenges formulated in conventional manner.

For administration by inhalation, the dimerizers for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the dimerizers and a suitable powder base such as lactose or starch.

The dimerizers may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The dimerizers may also be formulated in rectal compositions such as suppositories or retention enemas, e.g., containing conventional suppository bases such as cocoa butter or other glycerides.

In addition to the formulations described previously, the dimerizers may also be formulated as a depot preparation. Such long acting formulations may be administered by implantation (for example subcutaneously or intramuscularly) or by intramuscular injection. Thus, for example, the dimerizers may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Also encompassed is the use of adjuvants in combination with or in admixture with the dimerizers of the invention. Adjuvants contemplated include but are not limited to mineral salt adjuvants or mineral salt gel adjuvants, particulate adjuvants, microparticulate adjuvants, mucosal adjuvants, and immunostimulatory adjuvants. Adjuvants can be administered to a subject as a mixture with dimerizers of the invention, or used in combination with the dimerizers of the invention.

### 5.3. Treatment of Diseases and Disorders

The invention provides methods for treating any disease or disorder that is amenable to gene therapy. In one embodiment, “treatment” or “treating” refers to an amelioration of a disease or disorder, or at least one discernible symptom thereof. In another embodiment, “treatment” or “treating” refers to an amelioration of at least one measurable physical parameter associated with a disease or disorder, not necessarily discernible by the subject. In yet another embodiment, “treatment” or “treating” refers to inhibiting the progression of a disease or disorder, either physically, e.g., stabilization of a discernible symptom, physiologically, e.g., stabilization of a physical parameter, or both. Other conditions, including cancer, immune disorders, and veterinary conditions, may also be treated.

5.3.1. Target Diseases

Types of diseases and disorders that can be treated by methods of the present invention include, but are not limited to age-related macular degeneration; diabetic retinopathy; infectious diseases e.g., HIV pandemic flu, category 1 and 2 agents of biowarfare, or any new emerging viral infection; autoimmune diseases; cancer; multiple myeloma; diabetes; systemic lupus erythematosus (SLE); hepatitis C; multiple sclerosis; Alzheimer's disease; parkinson's disease; amyotrophic lateral sclerosis (ALS), huntington's disease; epilepsy; chronic obstructive pulmonary disease (COPD); joint inflammation, arthritis; myocardial infarction (MI); congestive heart failure (CHF); hemophilia A; or hemophilia B.

Infectious diseases that can be treated or prevented by the methods of the present invention are caused by infectious agents including, but not limited to, viruses, bacteria, fungi, protozoa, helminths, and parasites. The invention is not limited to treating or preventing infectious diseases caused by intracellular pathogens. Many medically relevant microorganisms have been described extensively in the literature, e.g., see C. G. A Thomas, Medical Microbiology, Bailliere Tindall, Great Britain 1983, the entire contents of which are hereby incorporated herein by reference.

Bacterial infections or diseases that can be treated or prevented by the methods of the present invention are caused by bacteria including, but not limited to, bacteria that have an intracellular stage in its life cycle, such as mycobacteria (e.g., Mycobacteria tuberculosis, M. bovis, M. avium, M. leprae, or M. africanum), rickettsia, mycoplasma, chlamydia, and legionella. Other examples of bacterial infections contemplated include but are not limited to infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g. Actinomyces and Clostridium species), Gram positive and negative coccal bacteria, Enterococcus species, Streptococcus species, Pneumococcus species, Staphylococcus species, Neisseria species. Specific examples of infectious bacteria include but are not limited to: Helicobacter pyloris, Borelia burgdorferi, Legionella pneumophilia, Mycobacteria tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Infectious virus of both human and non-human vertebrates, include retroviruses, RNA viruses and DNA viruses. Examples of virus that have been found in humans include but are not limited to: Retroviridae (e.g. human immunodeficiency viruses, such as HIV-1 (also referred to as HTL V-III, LA V or HTLV-III/LA V, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae, (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Parasitic diseases that can be treated or prevented by the methods of the present invention including, but not limited to, amebiasis, malaria, leishmania, coccidia, giardiasis, cryptosporidiosis, toxoplasmosis, and trypanosomiasis. Also encompassed are infections by various worms, such as but not limited to ascariasis, ancylostomiasis, trichuriasis, strongyloidiasis, toxoccariasis, trichinosis, onchocerciasis, filaria, and dirofilariasis. Also encompassed are infections by various flukes, such as but not limited to schistosomiasis, paragonimiasis, and clonorchiasis. Parasites that cause these diseases can be classified based on whether they are intracellular or extracellular. An “intracellular parasite” as used herein is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp.

Types of cancers that can be treated or prevented by the methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

5.3.2. Dosage and Mode of Administration of Viral Vectors

The replication-defective virus compositions of the invention can be administered to a human subject by any method or regimen known in the art. For example, the replication-defective virus compositions of the invention can be administered to a human subject by any method described in the following patents and patent applications that relate to methods of using AAV vectors in various therapeutic applications: U.S. Pat. Nos. 7,282,199; 7,198,951; U.S. Patent Application Publication Nos. US 2008-0075737; US 2008-0075740; International Patent Application Publication Nos. WO 2003/024502; WO 2004/108922; WO 20051033321, each of which is incorporated by reference in its entirety.

In an embodiment, the replication-defective virus compositions of the invention are delivered systemically via the liver by injection of a mesenteric tributary of portal vein. In another embodiment, the replication-defective virus compositions of the invention are delivered systemically via muscle by intramuscular injection in to e.g., the quadriceps or bicep muscles. In another embodiment, the replication-defective virus compositions of the invention are delivered to the basal forebrain region of the brain containing the nucleus basalis of Meynert (NBM) by bilateral, stereotactic injection. In another embodiment, the replication-defective virus compositions of the invention are delivered to the eNS by bilateral intraputaminal and/or intranigral injection. In another embodiment, the replication-defective virus compositions of the invention are delivered to the joints by intraarticular injection. In another embodiment, the replication-defective virus compositions of the invention are delivered to the heart by intracoronary infusion. In another embodiment, the replication-defective virus compositions of the invention are delivered to the retina by injection into the subretinal space.

In another embodiment, an amount of replication-defective virus composition is administered at an effective dose that is in the range of about 1.0×10⁸ genome copies (GC)/kilogram (kg) to about 1.0×GC/kg, and preferably 1.0×10¹¹ GC/kg to 1.0×10¹³

GC/kg to a human patient. Preferably, the amount of replication-defective virus composition administered is 1.0×10⁸ GC/kg, 5.0×10⁸ GC/kg, 1.0×10⁹ GC/kg, 5.0×10⁹ GC/kg, 1.0×10¹⁰ GC/kg, 5.0×10¹⁰ GC/kg, 1.0×10¹¹ GC/kg, 5.0×10¹¹ GC/kg, or 1.0×10¹² GC/kg, 5.0×10¹² GC/kg, 1.0×10¹³ GC/kg, 5.0×10¹³ GC/kg, 1.0×10¹⁴ GC/kg

These doses can be given once or repeatedly, such as daily, every other day, weekly, biweekly, or monthly, or until adequate transgene expression is detected in the patient. In an embodiment, replication-defective virus compositions are given once weekly for a period of about 4-6 weeks, and the mode or site of administration is preferably varied with each administration. Repeated injection is most likely required for complete ablation of transgene expression. The same site may be repeated after a gap of one or more injections. Also, split injections may be given. Thus, for example, half the dose may be given in one site and the other half at another site on the same day.

When packaged in two or more viral stocks, the replication-defective virus compositions can be administered simultaneously or sequentially. When two or more viral stocks are delivered sequentially, the later delivered viral stocks can be delivered one, two, three, or four days after the administration of the first viral stock. Preferably, when two viral stocks are delivered sequentially, the second delivered viral stock is delivered one or two days after delivery of the first viral stock.

Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal).

In one embodiment, the replication-defective virus compositions of the invention are delivered systemically via the liver by injection of a mesenteric tributary of portal vein at a dose of about 3.0×10¹² GC/kg. In another embodiment, the replication-defective virus compositions of the invention are delivered systemically via muscle by up to twenty intramuscular injections in to either the quadriceps or bicep muscles at a dose of about 5.0×10¹² GC/kg. In another embodiment, the replication-defective virus compositions of the invention are delivered to the basal forebrain region of the brain containing the nucleus basalis of Meynert (NBM) by bilateral, stereotactic injection at a dose of about 5.0×10¹¹ GC/kg. In another embodiment, the replication-defective virus compositions of the invention are delivered to the CNS by bilateral intraputaminal and/or intranigral injection at a dose in the range of about 1.0×10¹¹ GC/kg to about 5.0×10¹¹ GC/kg. In another embodiment, the replication-defective virus compositions of the invention are delivered to the joints by intra-articular injection at a dose of about 1.0×1011 GC/mL of joint volume for the treatment of inflammatory arthritis. In another embodiment, the replication-defective virus compositions of the invention are delivered to the heart by intracoronary infusion injection at a dose in the range of about 1.4×10¹¹ GC/kg to about 3.0×10¹² GC/kg. In another embodiment, the replication-defective virus compositions of the invention are delivered to the retina by injection into the subretinal space at a dose of about 1.5×10¹⁰ GC/kg.

Table 2 shows examples of transgenes that can be delivered via a particular tissue/organ by the PITA system of the invention to treat a particular disease.

TABLE 2
Treatment of Diseases

| Disease | Examples of transgenes | Target Tissue |
|---|---|---|
| Age relation macular | s-FIt-1, an anti-VEGF | Retina |
| degeneration | antibody such as | |
| | bevacizumab (Avastin), | |
| | ranibizumab (Lucentis), or a | |
| | domain antibody (dAB) | |
| HIV | a neutralizing antibody | Muscle and/or liver |
| | against HIV | |
| Cancer | Antiangiogenic agents (s- | Muscle and/or liver |
| | Fit-I, an anti-VEGF | |
| | antibody such as | |
| | bevacizumab (Avastin), | |
| | ranibizumab (Lucentis), or a | |
| | domain antibody (dAB); | |
| | cytokines that enhance | |
| | tumor immune responses, | |
| | anti-EGFR, IFN | |
| Autoimmune diseases, e.g., | Antibodies that interfere | Muscle and/or liver |
| arthritis, systemic lupus with | responses e.g., β-IFN; | |
| T cell activation; | adhesion molecule a4- | |
| erythematosus, psoriasis, | integrin antibody | |
| cytokines that bias immune | | |
| multiple sclerosis (MS) | | |
| Multiple myeloma | anti-CD20 antibody | Muscle and/or liver |
| Diabetes | GLP-1, IL-6 | Muscle and/or liver |
| Hepatitis C | β-IFN, shRNA targeting | Muscle and/or liver |
| | IRES | |
| Alzheimer's disease | NGF | Central nervous system |
| | | (CNS) |
| Amyotrophic lateral sclerosis | IGF-1 | CNS |
| (ALS) | | |
| Huntington's disease | NGF, BDNF AND CNTF, | CNS |
| | shRNA targeting mutant | |
| | Huntington | |
| Epilepsy | galanin, neuropeptide Y | CNS |
| | (NPY), glial cell line derived | |
| | neurotrophic factor | |
| | (GDNF) | |
| COPD | chemokines from IL 8 | Lung |
| | family, TNF antagonist | |
| Inflammatory arthritis | TNF antagonist, IL-1, | Joint |
| | anti-CD 20, IL-6, IL-1r | |
| | antagonist | |
| Myocardial infarction | Heme oxygenase-1 | Heart |
| Congestive heart failure | insulin like growth factor | Heart |
| | (IGF), hepatocyte growth | |
| | factor (HGF) | |
| Parkinson's Disease | GDNF, aromatic L-amino | CNS |
| | acid decarboxylase (ADCC), | |
| | NGF | |

In one embodiment a method for treating age-related macular degeneration in a human subject comprises administering an effective amount of a replication-defective virus composition, in which the therapeutic product is a VEGF antagonist.

In another embodiment, a method for treating hemophilia A in a human subject, comprises administering an effective amount of a replication-defective virus composition, in which the therapeutic product is Factor VIII or its variants, such as the light chain and heavy chain of the heterodimer and the B-deleted domain; U.S. Pat. No. 6,200,560 and U.S. Pat. No. 6,221,349). The Factor VIII gene codes for 2351 amino acids and the protein has six domains, designated from the amino to the terminal carboxy terminus as A1-A2-B-A3-C1-C2 [Wood et al, Nature, 312:330 (1984); Vehar et al., Nature 312:337 (1984); and Toole et al, Nature, 342:337 (1984)]. Human Factor VIII is processed within the cell to yield a heterodimer primarily comprising a heavy chain containing the A1, A2 and B domains and a light chain containing the A3, C1 and C2 domains. Both the single chain polypeptide and the heterodimer circulate in the plasma as inactive precursors, until activated by thrombin cleavage between the A2 and B domains, which releases the B domain and results in a heavy chain consisting of the A1 and A2 domains. The B domain is deleted in the activated procoagulant form of the protein. Additionally, in the native protein, two polypeptide chains (“a” and “b”), flanking the B domain, are bound to a divalent calcium cation. In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560]. Examples of naturally occurring and recombinant forms of Factor VII can be found in the patent and scientific literature including, U.S. Pat. No. 5,563,045, U.S. Pat. No. 5,451,521, U.S. Pat. No. 5,422,260, U.S. Pat. No. 5,004,803, U.S. Pat. No. 4,757,006, U.S. Pat. No. 5,661,008, U.S. Pat. No. 5,789,203, U.S. Pat. No. 5,681,746, U.S. Pat. No. 5,595,886, U.S. Pat. No. 5,045,455, U.S. Pat. No. 5,668,108, U.S. Pat. No. 5,633,150, U.S. Pat. No. 5,693,499, U.S. Pat. No. 5,587,310, U.S. Pat. No. 5,171,844, U.S. Pat. No. 5,149,637, U.S. Pat. No. 5,112,950, U.S. Pat. No. 4,886,876; International Patent Publication Nos. WO 94/11503, WO 87/07144, WO 92/16557, WO 91/09122, WO 97/03195, WO 96/21035, and WO 91/07490; European Patent Application Nos. EP 0 672 138, EP 0 270 618, EP 0 182 448, EP 0 162 067, EP 0 786 474, EP 0 533 862, EP 0 506 757, EP 0 874 057, EP 0 795 021, EP 0 670 332, EP 0 500 734, EP 0 232 112, and EP 0 160 457; Sanberg et al., XXth Int. Congress of the World Fed. Of Hemophilia (1992), and Lind et al., Eur. J. Biochem., 232:19 (1995).

In another embodiment, a method for treating hemophilia B in a human subject, comprises administering an effective amount of a replication-defective virus composition of, in which the therapeutic product is Factor IX.

In another embodiment, a method for treating congestive heart failure in a human subject, comprises administering an effective amount of a replication-defective virus composition, in which the therapeutic product is insulin like growth factor or hepatocyte growth factor.

In another embodiment, a method for treating a central nervous system disorder in a human subject, comprises administering an effective amount of a replication-defective virus composition, in which the therapeutic product is nerve growth factor.

### 5.4. Monitoring Transgene Expression and Undesired Side Effects

5.4.1. Monitoring Transgene Expression

After administration of the replication-defective virus compositions of the invention, transgene expression can be monitored by any method known to one skilled in the art. The expression of the administered transgenes can be readily detected, e.g., by quantifying the protein and/or RNA encoded by said transgene. Many methods standard in the art can be thus employed, including, but not limited to, immunoassays to detect and/or visualize protein expression (e.g., western blot, immunoprecipitation followed by sodium dodecyl sulfate polyacrylamide gel electrophoresis (SDS-PAGE), immunocytochemistry, immunohistochemical staining on sections etc) and/or hybridization assays to detect gene expression by detecting and/or visualizing respectively mRNA encoding a gene (e.g., northern assays, dot blots, in situ hybridization, etc.). The viral genome and RNA derived from the transgene can also be detected by Quantitative-PCR (Q-PCR). Such assays are routine and well known in the art. Immunoprecipitation protocols generally comprise lysing a population of cells in a lysis buffer such as RIP A buffer (1% NP-40 or Triton x-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G Sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32p or 1251) diluted in blocking buffer, washing the membrane in wash buffer, and detecting the presence of the antigen. One of skill in the art would be knowledgeable as to the parameters that can be modified to increase the signal detected and to reduce the background noise.

ELISAs generally comprise preparing antigen, coating the well of a 96 well microtiter plate with the antigen, adding the antibody of interest conjugated to a detectable agent such as an enzymatic substrate (e.g., horseradish peroxidase or alkaline phosphatase) to the well and incubating for a period of time, and detecting the presence of the antigen. In ELISAs the antibody of interest does not have to be conjugated to a detectable agent; instead, a second antibody (which recognizes the antibody of interest) conjugated to a detectable compound may be added to the well. Further, instead of coating the well with the antigen, the antibody may be coated to the well. In this case, a second antibody conjugated to a detectable agent may be added following the addition of the antigen of interest to the coated well. One of skill in the art would be knowledgeable as to the parameters that can be modified to increase the signal detected as well as other variations of ELISAs known in the art.

A phenotypic or physiological readout can also be used to assess expression of a transgene. For example, the ability of a transgene product to ameliorate the severity of a disease or a symptom associated therewith can be assessed. Moreover, a positron emission tomography (PET) scan and a neutralizing antibody assay can be performed.

Moreover, the activity a transgene product can be assessed utilizing techniques well-known to one of skill in the art. For example, the activity of a transgene product can be determined by detecting induction of a cellular second messenger (e.g., intracellular Ca2+, diacylglycerol, 1P3, etc.), detecting the phosphorylation of a protein, detecting the activation of a transcription factor, or detecting a cellular response, for example, cellular differentiation, or cell proliferation or apoptosis via a cell based assay. The alteration in levels of a cellular second messenger or phosphorylation of a protein can be determined by, e.g., immunoassays well-known to one of skill in the art and described herein. The activation or inhibition of a transcription factor can be detected by, e.g., electromobility shift assays, and a cellular response such as cellular proliferation can be detected by, e.g., trypan blue cell counts, ³H-thymidine incorporation, and flow cytometry.

5.4.2. Monitoring Undesirable Side Effects/Toxicity

After administration of a replication-defective virus composition of the invention to a patient, undesired side effects and/or toxicity can be monitored by any method known to one skilled in the art for determination of whether to administer to the patient a pharmaceutical composition comprising a dimerizer (described in Section 5.2.3) in order to ablate or excise a transgene or to ablate the transcript of the transgene, or inhibit its translation.

The invention provides for methods of determining when to administer a pharmacological agent for ablating the therapeutic product to a subject who received a replication-defective virus composition encoding a therapeutic product and an ablator, comprising: (a) detecting expression of the therapeutic product in a tissue sample obtained from the patient, and (b) detecting a side effect associated with the presence of the therapeutic product in said subject, wherein detection of a side effect associated with the presence of the therapeutic product in said subject indicates a need to administer the pharmacological agent that induces expression of the ablator.

The invention also provides methods for determining when to administer a pharmacological agent for ablating the therapeutic product to a subject who received a replication-defective virus composition encoding a therapeutic product and an ablator, comprising: detecting the level of a biochemical marker of toxicity associated with the presence of the therapeutic product in a tissue sample obtained from said subject, wherein the level of said marker reflecting toxicity indicates a need to administer the pharmacological agent that induces expression of the ablator. Biochemical markers of toxicity are known in the art, and include clinical pathology serum measures such as, but not limited to, markers for abnormal kidney function (e.g., elevated blood urea nitrogen (BUN) and creatinine for renal toxicity); increased erythrocyte sedimentation rate as a marker for generalized inflammation; low white blood count, platelets, or red blood cells as a marker for bone marrow toxicity; etc. Liver function tests (lft) can be performed to detect abnormalities associated with liver toxicity. Examples of such lfts include tests for albumin, alanine transaminase, aspartate transaminase, alkaline phosphatase, bilirubin, and gamma glutamyl transpeptidase.

The invention further comprises methods for determining the presence of DNA encoding the therapeutic gene product, its RNA transcript, or its encoded protein in a tissue sample from the subject subsequent to treatment with the pharmacological agent that induces expression of the ablator, wherein the presence of the DNA encoding the therapeutic gene product, its RNA transcript, or its encoded protein indicates a need for a repeat treatment with the pharmacological agent that induces expression of the ablator.

One undesired side effect that can be monitored in a patient that has received a replication-defective virus composition of the invention is an antibody response to a secreted transgene product. Such an antibody response to a secreted transgene product occurs when an antibody binds the secreted transgene product or to self antigens that share epitopes with the transgene product. When the transgene product is an antibody, the response is referred to as an “anti-idiotype” response. When soluble antigens combine with antibodies in the vascular compartment, they may form circulating immune complexes that are trapped nonspecifically in the vascular beds of various organs, causing so-called immune complex diseases, such as serum sickness, vasculitis, nephritis systemic lupus erythematosus with vasculitis or glomerulonephritis.

In another, more generalized undesirable immune reaction to the secreted transgene product, an antibody response to the transgene product results in a cross reacting immune response to one or more self antigens, causing almost any kind of autoimmunity. Autoimmunity is the failure of an the immune system to recognize its own constituent parts as self, which allows an immune response against its own cells and tissues, giving rise to an autoimmune disease. Autoimmunity to the transgene product of the invention can give rise to any autoimmune disease including, but not limited to, Ankylosing Spondylitis, Crohns Disease, Idiopathic inflammatory bowel disease, Dermatomyositis, Diabetes mellitus type-1, Goodpasture's syndrome, Graves' disease, Guillain-Barre syndrome (GBS), Anti-ganglioside, Hashimoto's disease, Idiopathic thrombocytopenic purpura, Lupus erythematosus, Mixed Connective Tissue Disease, Myasthenia gravis, Narcolepsy, Pemphigus vulgaris, Pernicious anaemia, Psoriasis, Psoriatic Arthritis, Polymyositis, Primary biliary cirrhosis, Rheumatoid arthritis, Sjogren's syndrome, Temporal arteritis (also known as “giant cell arteritis”), Ulcerative Colitis (one of two types of idiopathic inflammatory bowel disease “IBD”), Vasculitis, and Wegener's granulomatosis.

Immune complex disease and autoimmunity can be detected and/or monitored in patients that have been treated with replication-defective virus compositions of the invention by any method known in the art. For example, a method that can be performed to measure immune complex disease and/or autoimmunity is an immune complex test, the purpose of which is to demonstrate circulating immune complexes in the blood, to estimate the severity of immune complex disease and/or autoimmune disease, and to monitor response after administration of the dimerizer. An immune complex test can be performed by any method known to one of skill in the art. In particular, an immune complex test can be performed using anyone or more of the methods described in U.S. Pat. No. 4,141,965, U.S. Pat. No. 4,210,622, U.S. Pat. No. 4,210,622, U.S. Pat. No. 4,331,649, U.S. Pat. No. 4,544,640, U.S. Pat. No. 4,753,893, and U.S. Pat. No. 5,888,834, each of which is incorporated herein by reference in its entirety.

Detection of symptoms caused by or associated with anyone of the following autoimmune diseases using methods known in the art is yet another way of detecting autoimmunity or immune complex disease caused by a secreted transgene product that was encoded by a replication-defective virus composition administered to a human subject: Ankylosing Spondylitis, Crohns Disease, Idiopathic inflammatory bowel disease, Dermatomyositis, Diabetes mellitus type-I, Goodpasture's syndrome, Graves' disease, Guillain-Barre syndrome (GBS), Anti-ganglioside, Hashimoto's disease, Idiopathic thrombocytopenic purpura, Lupus erythematosus, Mixed Connective Tissue Disease, Myasthenia gravis, Narcolepsy, Pemphigus vulgaris, Pernicious anaemia, Psoriasis, Psoriatic Arthritis, Polymyositis, Primary biliary cirrhosis, Rheumatoid arthritis, Sjogren's syndrome, Temporal arteritis (also known as “giant cell arteritis”), Ulcerative Colitis (one of two types of idiopathic inflammatory bowel disease “IBD”), Vasculitis, and Wegener's granulomatosis.

A common disease that arises out of autoimmunity and immune complex disease is vasculitis, which is an inflammation of the blood vessels. Vasculitis causes changes in the walls of blood vessels, including thickening, weakening, narrowing and scarring. Common tests and procedures that can be used to diagnose vasculitis include, but are not limited to blood tests, such as erythrocyte sedimentation rate, C-reactive protein test, complete blood cell count and anti-neutrophil cytoplasmic antibodies test; urine tests, which may show increased amounts of protein; imaging tests such as X-ray, ultrasound, computerized tomography (CT) and magnetic resonance imaging (MRI) to determine whether larger arteries, such as the aorta and its branches, are affected; X-rays of blood vessels (angiograms); and performing a biopsy of part of a blood vessel. General signs and symptoms of vasculitis that can be observed in patients treated by the methods of the invention include, but are not limited to, fever, fatigue, weight loss, muscle and joint pain, loss of appetite, and nerve problems, such as numbness or weakness.

When administration of a replication-defective virus composition of the invention results in local transgene expression, localized toxicities can be detected and/or monitored for a determination of whether to administer to the patient a pharmaceutical composition comprising a dimerizer (described in Section 5.2.3) in order to ablate or excise a transgene or to ablate the transcript of the transgene, or inhibit its translation. For example, when administering to the retina a replication-defective virus composition that comprises a transgene unit encoding a VEGF inhibitor for treatment of age-related macular degeneration, it is believed that VEGF may be neuroprotective in the retina, and inhibiting it could worsen eye-sight due to drop out of ganglion cells. Thus, after administration of such a replication-defective virus composition, eye-sight can be regularly monitored and ganglion cell drop out can be detected by any method known the art, e.g., noninvasive imaging of retina. Moreover, VEGF inhibition may also depleted necessary micro vasculature in the retina, which can be monitored using fluorescien angiography or any other method known in the art.

In general, side effects that can be detected/monitored in a patient after administration of a replication-defective virus of the invention for a determination of whether to administer a pharmaceutical composition comprising a dimerizer (described in Section 5.2.3) to the patient, include, but are not limited to bleeding of the intestine or any organ, deafness, loss of eye-sight, kidney failure, dementia, depression, diabetes, diarrhea, vomiting, erectile dysfunction, fever, glaucoma, hair loss, headache, hypertension, heart palpitations, insomnia, lactic acidosis, liver damage, melasma, thrombosis, priapism rhabdomyolysis, seizures, drowsiness, increase in appetite, decrease in appetite, dizziness, stroke, heart failure, or heart attack. Any method commonly used in the art for detecting the foregoing symptoms or any other side effects can be employed.

Ablator Therapy; Once it has been determined that a transgene product that was delivered to a patient by a method of the invention has caused undesirable side effects in a patient, a pharmaceutical composition comprising a dimerizer can be administered to a patient using any of the regimens, modes of administrations, or doses described in Section 5.2.3 herein.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

### 6. EXAMPLE 1

### Manufacturing of Recombinant AAV Vectors at Scale

This example describes a high yielding, recombinant AAV production process based upon poly-ethylenimine (PEI)-mediated transfection of mammalian cells and iodixanol gradient centrifugation of concentrated culture supernatant. AAV vectors produced with the new process demonstrate equivalent or better transduction both in vitro and in vivo when compared to small scale, cesium chloride (CsCl) gradient-purified vectors. In addition, the iodixanol gradient purification process described effectively separates functional vector particles from empty capsids, a desirable property for reducing toxicity and unwanted immune responses during pre-clinical studies.

### 6.1. Introduction

In recent years the use of recombinant adeno-associated viral (rAAV) vectors for clinical gene therapy applications has become widespread and is largely due to the demonstration of long-term transgene expression from rAAV vectors in animal models with little associated toxicity and good overall safety profiles in both pre-clinical and clinical trials (Snyder and Flotte 2002; Moss et al. 2004; Warrington and Herzog 2006; Maguire et al. 2008; Mueller and Flotte 2008; Brantly et al. 2009). Most early AAV gene therapy studies were performed with serotype 2 (“AAV2”) vectors, but vector systems based on other AAV serotypes with more efficient gene delivery and different tissue specificity are currently in human trials and their use will likely increase (Brantly et al. 2009; Neinhuis 2009).

A major requirement for development and eventual marketing of a gene therapy drug is the ability to produce the gene delivery vector at a sufficient scale. In the past this requirement has been a barrier to the successful application of rAAV vectors but more recently several innovative production systems have been developed which are compatible with large scale production for clinical application. These new systems use adenovirus, herpesvirus and baculovirus hybrids to deliver the rAAV genome and trans-acting helper functions to producer cells and have been recently reviewed (Clement et al. 2009; Virag et al. 2009; Zhang et al. 2009). The ease of introduction of the required genetic elements to the producer cell line through rAAV hybrid virus infection permits efficient rAAV vector production and importantly, up-scaling of the process to bioreactors. These systems are particularly suited to final clinical candidate vectors, but because of the need to make hybrid viruses for each vector, they are less suited to early development and pre-clinical studies where several combinations of transgene and vector serotype may need to be evaluated.

While much pre-clinical rAAV-based gene therapy work has been performed in mice, results obtained in larger animals are often considered more predictive of actual clinical outcomes. Large animal studies require higher rAAV vector doses and to satisfy these demands, a versatile production system which can rapidly produce a variety of test vectors at scale without the need for time-consuming production of intermediates is required. Transient transfection by calcium phosphate co-precipitation of plasmid DNAs containing the AAV vector genome, the AAV capsid gene and the trans-acting helper genes into HEK 293 cells (a process known as “triple transfection”), has long been the standard method to produce rAAV in the research laboratory (Grimm et al. 1998; Matsushita et al. 1998; Salvetti et al. 1998; Xiao et al. 1998). Transfection-based methods remain the most versatile of all rAAV production techniques and permit simultaneous manufacture of different rAAV vectors. However, triple transfection has generally not been considered ideal for large scale rAAV production due to a lack of compatibility with suspension culture systems. Recently, however, some promising results using poly-ethylenimine (PEI) as a transfection reagent have demonstrated the production of rAAV2 vectors in mammalian cell suspension culture with unpurified yields of 1-3×10¹³ vector particles per liter, which are comparable to yields from attached mode (cells grown as monolayer on culture dish) transfection systems (Durocher et al. 2007; Hildinger et al. 2007). The advantages of PEI-based transfection are that it can also be performed in serum-free medium without the need for the media exchanges which are typically required with conventional calcium phosphate-mediated transfection (Durocher et al. 2007). These features translate into lower cost and the elimination of concerns surrounding animal-derived serum such as the presence of prions and other adventitious agents.

A further impediment to the scale-up of rAAV vector production occurs during downstream processing of the vector. At small scale, the most prevalent method used for rAAV vector purification involves multiple rounds of overnight cesium chloride (CsCl) gradient centrifugation (Zolotukhin et al. 1999). This purification method can be performed easily with standard laboratory equipment, is generally high-yielding and when performed carefully gives vector of reasonable purity. The drawbacks of this technique, however, are first, that prolonged exposure to CsCl has been reported to compromise the potency of rAAV vectors (Zolotukhin et al. 1999; Auricchio et al. 2001; Brument et al. 2002) and second, that the gradients have a limited loading capacity for cell lysate which can in turn limit rAAV purification scale-up. An alternative gradient medium, iodixanol, has also been used to purify rAAV vectors (Hermens et al, 1999; Zolotukhin et al. 1999). This isotonic medium was developed originally as a contrast agent for use during coronary angiography and the low associated toxicity and relative inertness are advantages over CsCl from both safety and vector potency points of view (Zolotukhin et al. 1999). However iodixanol shares the same drawback as CsCl in that the loading capacity for rAAV production culture cell lysate and thus the scalability of rAAV purification are limited. To overcome these gradient-specific constraints, researchers have gravitated towards ion exchange chromatography and, more recently, affinity purification using single-domain heavy chain antibody fragments to purify AAV at scale (Auricchio et al. 2001; Brument et al. 2002; Kaludov et al. 2002; Zolotukhin et al. 2002; Davidoff et al. 2004; Smith et al. 2009). These techniques enhance AAV yields, scalability and purity. However, there remain vector related impurities such as empty capsids, which are not generally separated from fully functional vector particles using chromatography-based techniques. While some progress has been made using AAV2 vectors to develop ion exchange-based resolution of empty and full vector particles (Qu et al. 2007; Okada et al. 2009), CsCl gradient centrifugation remains the best-characterized method for removing empty particles from rAAV vector preparations.

Recently it was observed that, in contrast to AAV2, most other AAV serotypes are primarily released into the media of calcium phosphate-transfected production cultures and not retained in the cell lysate (Vandenberghe et al. 2010). Since this distribution occurs in the absence of cell lysis, it was reasoned that the production culture media would represent a relatively pure source of rAAV vector and that the lower level of cellular contaminants may improve the loading capacity and resolution of purification gradients.

Described in this example is a scaled rAAV production method suitable for large animal studies, which is based upon PEI transfection and supernatant harvest. The method is high yielding, versatile for the production of vectors with different serotypes and transgenes, and simple enough that it may be performed in most laboratories with a minimum of specialized techniques and equipment. In addition, this example demonstrates the use of iodixanol gradients for the separation of genome-containing vectors from empty particles.

### 6.2. Materials and Methods

6.2.1. Cell Culture

Late passage HEK293 cell cultures were maintained on 15 cm plates in DMEM (Mediatech Inc, Manassas, V A) with the addition 10% fetal bovine serum (FBS; Hyclone laboratories Inc, South Logan, Utah). The cells were passaged twice weekly to maintain them in exponential growth phase. For small scale transfections, 1×10⁶ HEK 293 cells were seeded per well of 6 well plates and 1.5×10⁷ cells were seeded into 15 cm dishes. For large scale production, HEK 293 cells from sixteen confluent 15 cm plates were split into two 10 layer cell stacks (Corning Inc., Corning, N.Y.) containing one liter of DMEM/10% FBS four days prior to transfection. The day before transfection, the two cell stacks were trypsinized and the cells resuspended in 200 mL of medium. Cell clumps were allowed to settle before plating 6.3×10⁸ cells into each of six cell stacks. The cells were allowed attach for 24 hours prior to transfection. Confluency of the cell stacks was monitored using a Diaphot inverted microscope (Nikon Corp.) from which the phase contrast hardware had been removed in order to accommodate the cell stack on the microscope stage.

6.2.2. Plasmids

The plasmids used for all transfections were as follows:
1) cis plasmid pENNAAVCMVeGFP.RBG (also referred to as “AAV cis”), which contains an eGFP expression cassette flanked by AAV2 ITRs;
2) trans plasmids pAAV2/1, pAAV2/6. pAAV217, pAAV2/8 and pAAV2/9 (also referred to as “AAV trans”), which contain the AAV2 rep gene and capsid protein genes from AAV1, 6, 7, 8 and respectively; and
3) adenovirus helper plasmid pAdΔF6.
15 to 50 mg lots of >90% supercoiled plasmid were obtained (Puresyn Inc., Malvern, Pa.) and used for all transfections.

6.2.3. Calcium Phosphate Transfection

Small scale calcium phosphate transfections were performed by triple transfection of AAV cis, AAV trans and adenovirus helper plasmids as previously described (Gao et al. 2002). Briefly, the medium on 85-90% confluent HEK 293 monolayers in 6 well plates was changed to DMEM/10% FBS two hours prior to transfection. Plasmids in the ratio of 2:1:1 (1.73 μg adenovirus helper/0.86 mg cis/0.86 μg trans per well) were calcium phosphate-precipitated and added dropwise to plates. Transfections were incubated at 37° C. for 24 hours, at which point the medium was changed again to DMEM/10% FBS. The cultures were further incubated to 72 hours post infection before harvesting the cells and medium separately. For large scale transfection of cell stacks, the plasmid ratio was kept constant but all reagent amounts were increased by a factor of 630. The transfection mix was added directly to 1 L DMEM/10% FBS and this mixture was used to replace the medium in the cell stack. The medium was changed at 24 hours post-transfection. Cells and medium were harvested after 72 hours or 120 hours post-transfection either directly or after further incubation for 2 hours in the presence of 500 mM NaCl. In cases where vector present in the cells was to be quantified, the cells were released by trypsinization and lysates formed by 3 freeze/thaw cycles.

6.2.4. Small Scale Vector Preparation

Forty 15 cm plates were transfected by the calcium phosphate method and cell lysates prepared 72 hours post-transfection with 3 successive cycles of freeze/thaw (−80° C./37° C.). Cell lysates were purified with two rounds of cesium chloride (CsCl) centrifugation and pure gradient fractions were concentrated and desalted using ultra 15 centrifugal concentrator devices (Amicon; Millipore Corp., Bedford Mass.).

6.2.5. Small Scale Polyethylenimine Transfection

For polyethylenimine (PEI)-based triple transfections of HEK 293 cells in six well plates, the same plasmid amounts were used as described for calcium phosphate transfections. PEI-max (Polysciences Inc., Warrington, Pa.) was dissolved at 1 mg/mL in water and the pH adjusted to 7.1. 2 μg of PEI were used per μg of DNA transfected. PEI and DNA were each added to 100 μL of serum-free DMEM and the two solutions combined and mixed by vortexing. After 15 minutes of incubation at room temperature the mixture was added to 1.2 mL serum free medium and used to replace the medium in the well. No further media change was carried out. For 15 cm plates, the plasmid ratio was kept constant but the amount of plasmid and other reagents used were increased by a factor of 15.

6.2.6. Large Scale Polyethylenimine Transfection

Large scale PEI-based transfections were performed in 10 layer cell stacks containing 75% confluent monolayers of HEK 293 cells. Plasmids in the ratio of 2:1:1 (1092 μg adenovirus helper/546 μg cis/546 μg trans per cell stack) were used. The PEI-max: DNA ratio was maintained at 2:1 (weight/weight). For each cell stack, the plasmid mix and PEI were each added to a separate tube containing serum-free DMEM (54 mL total volume). The tubes were mixed by vortexing and incubated for 15 minutes at room temperature after which the mixture was added to 1 liter of serum-free DMEM containing antibiotics. The culture medium in the stack was decanted, replaced by the DMEM/PEI/DNA mix and the stack incubated in a standard 5% CO₂, 37° C. incubator. At 72 hours post-transfection, 500 mL of fresh serum free-DMEM was added and the incubation continued to 120 hours post-transfection. At this point, Bensonaze (EMD Chemicals, Gibbstown, N.J.) was added to the culture supernatant to 25 units/mL final concentration and the stack re-incubated for 2 hours. NaCl was added to 500 mM and the incubation resumed for an additional 2 hours before harvest of the culture medium (at this point the culture medium was called the “downstream feedstock”). In cases where cell associated vector was to be quantified, the cells were released by trypsinization and lysates were formed by three sequential freeze/thaw cycles (−80° C./37° C.).

6.2.7. Downstream Processing

10 liters of feedstock culture medium from six cell stacks was clarified through a 0.5 μm Profile II depth filter (Pall Corp., Fort Washington, N.Y.) into a 10 liter allegro media bag (Pall Corp., Fort Washington, N.Y.). The clarified feedstock was then concentrated by tangential flow filtration using a Novaset-LS LVH holder with customized ¼″ ID tubing and ports (TangenX Technology Corp., Shrewsbury, Mass.) and a 0.1 m² Sius-LS single use TFF screen channel cassette with a 100 kDa MWCO HyStream membrane (TangenX Technology Corp., Shrewsbury, Mass.). A 125-fold concentration to 85 mL was performed according to the manufacturer's recommendations with a transmembrane pressure of 10-12 psi maintained throughout the procedure. The TFF filter was discarded after each run and the system sanitized with 0.2 N NaOH between runs. The concentrated feedstock was reclarified by centrifugation at 10,500×g and 15° C. for 20 minutes and the supernatant carefully removed to a new tube. Six iodixanol step gradients were formed according to the method of Zoltukinin et al. (Zolotukhin et al. 1999) with some modifications as follows: Increasingly dense iodixanol (Optiprep; Sigma Chemical Co., St Louis, Mo.) solutions in PBS containing 10 mM magnesium chloride and 25 mM potassium were successively underlayed in 40 mL quick seal centrifuge tubes (Beckman Instruments Inc., Palo Alto, Calif.). The steps of the gradient were 4 mL of 15%, 9 mL of 25%, 9 mL of 40% and 5 mL of 54% iodixanol. 14 mL of the clarified feedstock was then overlayed onto the gradient and the tube was sealed. The tubes were centrifuged for 70 minutes at 350,000×g in a 70 Ti rotor (Beckman Instruments Inc., Palo Alto, Calif.) at 18° C. and the gradients fractionated through an 18 gauge needle inserted horizontally approximately 1 cm from the bottom of the tube. Fractions were diluted 20-fold with water into a UV transparent 96 well plate (Corning Inc., Corning, N.Y.) and the absorbance measured at 340 nm. A spike in OD₃₄₀ readings indicated the presence of the major contaminating protein band and all fractions below this spike were collected and pooled. Pooled fractions from all six gradients were combined, diafiltered against 10 volumes of the final formulation buffer (PBS/35 mM NaCl) and concentrated 4-fold to approximately 10 mL by tangential flow filtration according to the manufacturer's instructions using a 0.01 m² single use Sius TFF cassette with a 100 kDa MWCO Hystream screen channel membrane (TangenX Technology Corp., Shrewsbury, Mass.) and a Centramate LV cassette holder (Pall Corp., Fort Washington, N.Y.). A transmembrane pressure of 10 was maintained throughout the process. The holdup volume of the apparatus was kept low using minimal lengths of platinum cured silicone tubing (1.66 mm inner diameter, Masterflex; Cole Palmer Instrument Co., Vernon Hills, Ill.). In addition, all wetable parts were pre-treated for 2 hours with 0.1% Pluronic F68 (Invitrogen Corp., Carlsbad, Calif.) in order to minimize binding of the vector to surfaces. The TFF filter was discarded after each run and the system sanitized with 0.2 N NaOH between runs. Glycerol was added to the diafiltered, concentrated product to 5% final and the preparation was aliquoted and stored at −80° C.

6.2.8. Vector Characterization

DNase I-resistant vector genomes were titered by TaqMan PCR amplification (Applied Biosystems Inc., Foster City, Calif.), using primers and probes directed against the polyadenylation signal encoded in the transgene cassette. The purity of gradient fractions and final vector lots were evaluated by SDS polyacrylamide gel electrophoresis (SDSPAGE) and the DNA visualized using SYPRO ruby stain (Invitrogen Corp., Carlsbad, Calif.) and UV excitation. Purity relative to non-vector impurities visible on stained gels was determined using Genetools software (Syngene, Frederick, Md.). Empty particle content of vector preparations was assessed by negative staining and electron microscopy. Copper grids (400-mesh coated with a formvar/thin carbon film; Electron Microscopy Sciences, Hatfield, P A) were pre-treated with 1% Alcian Blue (Electron Microscopy Sciences, Hatfield, Pa.) and loaded with 5 μl of vector preparation. The grids were then washed, stained with 1% uranyl acetate (Electron Microscopy Sciences, Hatfield, Pa.) and viewed using a Philips CM100 transmission electron microscope.

Empty-to-full particle ratios were determined by direct counting of the electron micrographs.

6.2.9. Relative Vector Potency Assessment

Early passage HEK 293 cells were plated to 80% confluency in 96 well plates and infected with AAV vector at an MOI of 10,000 in the presence of wild type adenovirus type 5 (MOI: 400). 48 hours post-infection, GFP fluorescent images were captured digitally and the fluorescent intensity quantified as described previously (Wang et al. 2010) using ImageJ software (Rasband, 19997-2006, National Institutes of health, Bethesda, Md., http://rsb.info.nih.gov/ij/). For in vivo analysis of transduction, C57BL6 mice were injected i.v. with 1×10¹¹ genome copies of AAV vector. The animals were necropsied 9 days post-injection, the livers sectioned and imaged for GFP fluorescence as described previously (Wang et al. 2010) and fluorescent intensity quantified using ImageJ software.

### 6.3. Results of Comparison of Transfection Reagents for r AAV Production

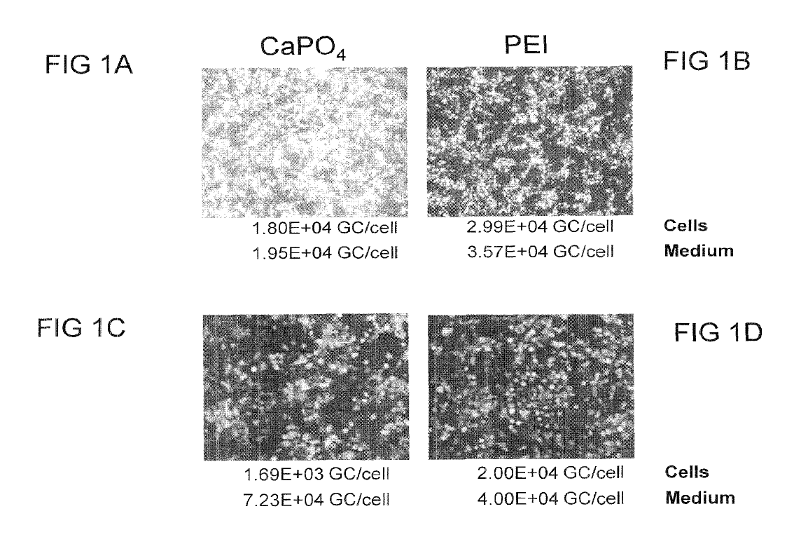

A standard upstream method for producing rAAV vectors at small scale (total yield: about 1-2×10¹³ genome copies (GC)) is based upon calcium phosphate-mediated triple transfection of HEK 293 cells in forty 15 cm tissue culture plates. While this method reproducibly yields vectors of various AAV serotypes with good titers in both the cell pellet and the culture medium (Vandenberghe et al. 2010), it is technically cumbersome, requires the presence of animal serum and involves two media changes. For scaled rAAV production, it was reasoned that a less complicated, more robust transfection agent such as polyethylenimine (PEI) may be advantageous. The production of rAAV7 vector carrying an eGFP expression cassette (rAAV7-eGFP) following either calcium phosphate or PEI-mediated triple transfection, was quantified by qPCR of DNase-resistant vector genomes in both cells and media of six-well plate HEK293 production cultures (FIGS. 1A-1D). With either transfection method, rAAV7-eGFP production was found to partition equally between the cells and culture media at similar levels, despite stronger expression of the eGFP transgene in the calcium phosphate-transfected cells. These results indicate that transgene expression levels in the production culture are not predictive of rAAV production yields and that rAAV7-eGFP is released to the culture medium at similar levels irrespective of the transfection technique.

6.3.1.1. Effect of Serotype and Salt Addition on rAAV Release to the Culture Medium

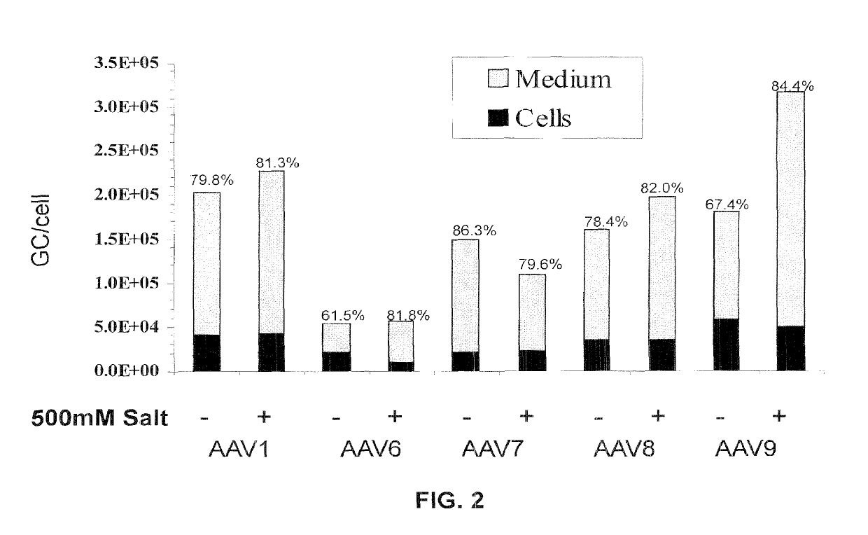

Having established the release of rAAV7-eGFP to the culture media following PEI triple transfection, an immediate goal was to demonstrate similar release with other AAV serotypes. In addition, a goal was to see if the 45% of detectable vector that remained associated with the cells (FIGS. 1A-D) could be moved into the culture medium. By postponing the harvest until 120 hours post-PEI transfection, as opposed to the standard 72 hours, the total vector in the culture medium was found to be doubled (data not shown). Adopting this strategy, 15 cm plates of HEK 293 cells were triple-transfected using PEI. Trans plasmids encoding 5 different AAV serotype capsid genes were included in the various transfection mixes and, following a 120 hour incubation, the culture medium and cells were harvested either immediately or 2 hours after addition of 500 mM NaCl. The encapsidated AAV genomes in the cell lysates and culture media were then quantified by qPCR (FIG. 2). Each of the five AAV serotypes tested was released to the supernatant after five days of incubation without salt addition at levels between 61.5% and 86.3% of the total GC yield. This result confirmed the observation during early development runs that increased incubation time post-transfection leads to higher titers of AAV vector in the culture medium. Incubation of production cultures with salt has been demonstrated to cause release of AAV2 to the supernatant, presumably through a mechanism mediated by cellular stress (Atkinson et al. 2005). The high salt incubation performed here led to a further approximately 20% GC release of AAV6 and AAV9 vectors to the culture medium, but elicited very little change with the other serotypes.

6.3.1.2. Effect of Scale-Up on rAAV7 Vector Yields

A goal of this study was to develop a scaled AAV production system that could be performed in most laboratories using standard equipment to support large animal preclinical studies. Hence, Corning 10 layer cell stacks were chosen to scale-up the PEI-based transfection, since this type of tissue culture vessel can be accommodated by standard laboratory incubators. Initially, a single 10-layer cell stack was seeded with 6.3×10⁸ HEK 293 cells such that the monolayers would be 75% confluent the next day. In order to assess the confluency of the bottom HEK293 monolayer prior to transfection, a standard laboratory microscope was adapted by removing the phase contrast hardware such that the cell stacks could be accommodated. One cell stack was triple transfected with the relevant plasmids to produce AAV7-eGFP vector using either calcium phosphate or PEI (see Materials and Methods) and then incubated to 120 hours post-infection prior to quantification of DNase-resistant vector genomes in both cells and media. Per cell yields from the PET transfected cell stack were similar to those obtained previously in six well and 15 cm plates (FIG. 1A-D, FIG. 2). The overall yield from the culture medium in this experiment was 2.2×10¹³ GC per cell stack. The calcium phosphate transfected stack produced significantly lower vector yields per cell than observed previously in plates and this effect may result from a lack of diffusion of CO₂ into the central areas of the cell stack. Based upon the 10 layer cell stack transfection results, PEI was chosen as the transfection reagent for further development of the scaled procedure.

6.3.1.3. Downstream Processing of the rAAV7-eGFP Production Culture Media

A goal of developing the scaled production process was to maintain flexibility such that any AAV vector could be purified by a generic method. Separation of vector from contaminants based on density and size are purification methods that can be applied to multiple vector serotypes. Hence, the rAAV7 vector in the culture medium was concentrated by Tangential flow filtration (TFF) to volumes small enough to permit purification over iodixanol density gradients. Pre-clarification of the production culture medium through a 0.5 μm depth filter was done to remove cellular debris and detached cells and to prevent clogging of the TFF membrane. A 130-fold concentration was then achieved using a disposable, 100 kDa cut-off screen channel TFF membrane while maintaining a transmembrane pressure of 10-12 psi throughout the process. The disposability of the membrane avoided the need to de-foul and sanitize between runs and therefore added to the reproducibility of the process. The production culture medium was treated with nuclease (Benzonase) to degrade contaminating plasmid and cellular DNA, and 500 mM salt was added prior to concentration to minimize aggregation of the vector to both itself (Wright et al. 2005) and to contaminating proteins during processing. These two treatments were subsequently determined to increase recoveries from the iodixanol gradient (data not shown). During development of the downstream process and performance of full scale pilot runs, no significant loss of vector was observed at any point due to the concentration process (see Table 3 below).

TABLE 3
In-process and final yields (GC) of AAV vector pilot production runs

| | | TFFI/Iodixanol Gradient | | | | TFF1I/Buffer Exchange | | |
|---|---|---|---|---|---|---|---|---|
| | | | TFF I | Fraction | % | Final | % of Iodixanol | Process |
| Pilot # | Serotype Transgene | Feedstock | Retentate | Pool | Feedstock | Product | Pool | Yield |
| 1 | AAV9 EGFP | 6.41E+14 | 3.58E+14 | 2.24E+14 | 42.15% | 1.82E+14 | 81.34% | 28.46% |
| 8 | AAV9 EGFP | 1.03E+15 | 2.26E+14* | 1.05E+14 | 10.20% | 6.66E+13 | 63.57% | 6.48% |
| 9 | AAV9 EGFP | 3.12E+14 | 3.76E+14 | 1.63E+14 | 52.33% | 8.38E+13 | 51.37% | 26.88% |
| 10 | AAV8 EGFP | 9.83E+14 | 1.22E+15 | 4.32E+14 | 43.97% | 2.66E+14 | 61.50% | 27.04% |
| 11 | AAV8 EGFP | 9.24E+14 | 1.01E+15 | 3.26E+14 | 35.29% | 2.00E+14 | 61.27% | 21.62% |
| 12 | AAV8 EGFP | 1.51E+15 | 1.57E+15 | 6.06E+14 | 40.19% | 3.67E+14 | 60.58% | 24.35% |
| 5 | AAV6 EGFP | 3.35E+13 | 5.69E+13 | 6.37E+12 | 22.92% | 2.48E+12 | 38.84% | 7.39% |
| 7 | AAV6 EGFP | 1.01E+14 | 1.32E+14 | 1.57E+13 | 15.58% | 4.58E+12 | 29.12% | 4.54% |

*Loss due to mechanical failure

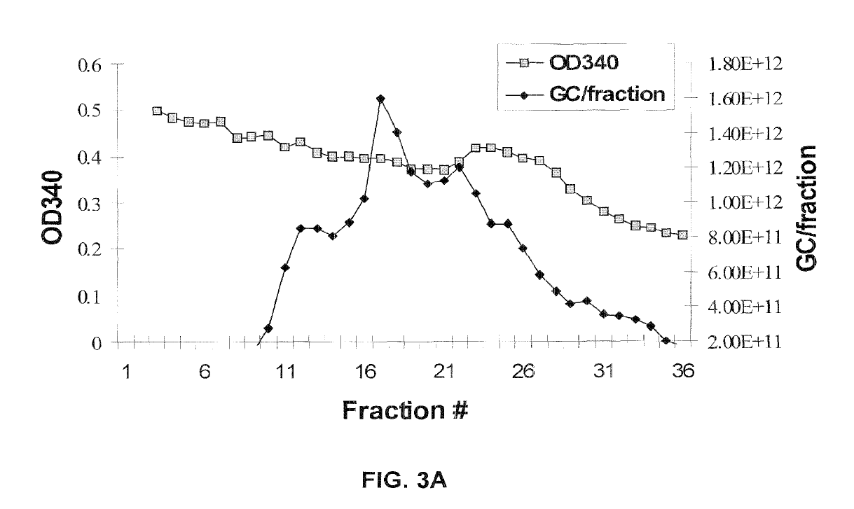

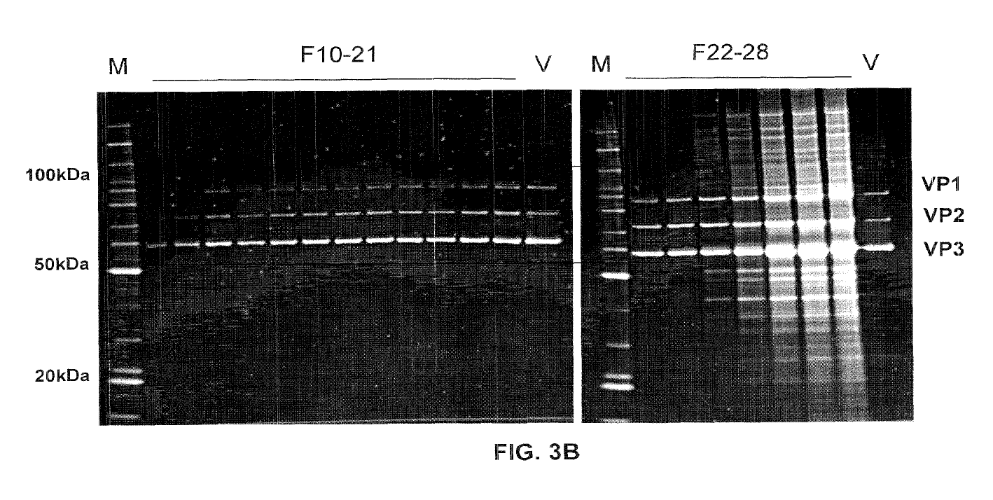

Iodixanol gradient purification of AAV vectors has been fully described (Zolotukhin et al. 1999) and the step gradient used here is adapted from this work. The volumes of the gradient layers were modified in order to achieve better resolution of vector from contaminants (see Materials and Methods). Fourteen milliliters of TFF retentate containing concentrated AAV7-eGFP vector from the production culture medium of one cell stack were loaded onto a 27 mL iodixanol step gradient and centrifuged for 1 hour at 350,000×g. The gradient was then fractionated from the bottom of the tube and the fractions (275 μL) analyzed for vector content, iodixanol concentration and vector purity using qPCR, optical density at 340 nm (Schroder et al. 1997) and SDS-PAGE, respectively. Representative profiles of one such gradient are shown in FIG. 3. A linear gradient of iodixanol concentration indicated by the decreasing OD340 readings was observed up until fraction 22. After this point, the readings increased (FIG. 3A) and corresponded to a spike in contaminating protein visualized by SDS-PAGE (FIG. 3B) and by the naked eye in the form of a thin band present in the gradient. The OD₃₄₀ spike was likely due to overlapping absorbance of protein and iodixanol at this wavelength and this phenomenon provided an accurate and reproducible method of detecting the emergence of the contaminating protein band.

The peak of vector genomes was observed towards the bottom third of the gradient between fractions 12 and 22 at an OD₃₄₀-extrapolated iodixanol concentration range of 1.31 g/mL to 1.23 g/mL (FIG. 3A), just below the start of the contaminating cellular protein band (fractions 23 to 28). This peak coincided with those fractions containing pure vector particles as judged by the presence of AAV capsids proteins without contaminating cellular protein (FIG. 3B). Approximately 50% of the vector genomes consistently co-migrated with the contaminating protein and could not be resolved despite attempts to do so using different iodixanol concentrations, spin times, salt concentrations and detergents (data not shown). Despite loading equal genome copies of each fraction (10¹⁰ GC) on the SDS-PAGE gels, fractions 26, 27 and 28 contained elevated levels of the capsid proteins VP1, 2 and 3 (FIG. 3B). This result suggested the presence in these fractions of either empty capsids or capsid assembly intermediates with no associated or packaged genome. It is concluded that the iodixanol gradient is capable of separating full and empty rAAV particles, a result that previously had not been formally demonstrated.

6.3.1.4. Large Scale Pilot Production Run Recoveries and Yields

The development work described above demonstrates that pure rAAV7 vector could be produced at high titer from a single cell stack using a combination of PEI transfection and iodixanol gradient purification. In order to characterize the production process and demonstrate reproducibility and applicability to other AAV serotypes, full scale pilot production runs were initiated, each using six cell stacks. The goal for each run was to produce in excess of 10¹⁴ GC of final purified vector; the final process employed is summarized in Table 4 below and fully detailed in Materials and Methods.

TABLE 4
Summary of the large scale vector production process. Major
process steps and corresponding timeline are shown.

| Day | |
|---|---|
| 1 | Seed 6 cell stacks HEK 293 cells |

↓

| | |
|---|---|
| 2 | PEI based triple transfection (cis, trans, helper) |
| | Incubate serum-free, 37° C., 5 days |

↓

| | |
|---|---|
| 6 | Benzonase treat supernatant |
| | Adjust salt to 500 mM (101 final) |
| | Clarify |

↓

| | |
|---|---|
| 6 | TFF 1 (100 kDa MWCO) |
| | Concentrate 125-fold |

↓

| | |
|---|---|
| 7 | Clarify |
| | Load to 6x iodixanol step gradients |
| | 1 hr, 350k × g |

↓

| | |
|---|---|
| 7 | Harvest fractions and monitor OD₃₄₀ |
| | Pool pure window |

↓

| | |
|---|---|
| 7 | TFF 2 (100 kDa MWCO) |
| | Buffer exchange to final formulation |
| | Concentrate (1-4 × 10¹³/mL) |

Three runs each of rAAV8-eGFP and rAAV9-eGFP were performed along with two runs of rAAV6-eGFP. In-process samples were taken at various stages to assess vector loss throughout as follows: 1) feedstock samples were taken following treatment of the culture medium with benzonase/0.5 M salt and clarification; 2) retentate samples were taken following TFF concentration; 3) iodixanol gradient fraction samples were taken after gradient harvest and fraction pooling; and 4) final product samples were taken after buffer exchange and final concentration by a second TFF procedure. The recoveries of encapsidated vector genome copies at each of these stages for the various runs are listed in Table 3.

The mean recovery of rAAV8 and rAAV9 vector in the feedstock was 9.0×10¹⁴ GC, whereas for rAAV6 vectors the mean recovery was 6.7×10¹³ GC. Similar low yields of rAAV6 vectors were seen in transfections during development (FIG. 2) and are also consistently observed in a standard small scale AAV production process.

A 125-fold concentration of the feedstock from 10 L to 85 mL (Table 3: TFFI retentate) resulted in 110 loss of vector except for one instance where the loss was due to a mechanical failure. In several cases, there was an apparent increase in yield upon concentration, but this was attributed to error introduced by extra dilution of the retentate, which was necessary to overcome inhibition of the qPCR reaction. As was the case during development runs, there was loss of the vector during pilot iodixanol purification runs with recoveries between 35% and 50% of feedstock for AAV8 and AAV9 vectors. More loss was seen with AAV6 vectors during purification (80-85% of feedstock). Final concentration and buffer exchange led to further losses, although this was most pronounced with AAV6 vectors, possibly because of the lower titer of the starting material and therefore a larger fraction of vector absorbed to the surfaces of the TFF apparatus. Excluding the run where mechanical loss occurred, the average overall process yield for AAV8 and AAV9 vectors was 2.2×10¹⁴ GC (approximately 26% of feedstock).

6.3.1.5. Characterization of Large Scale Production Lots

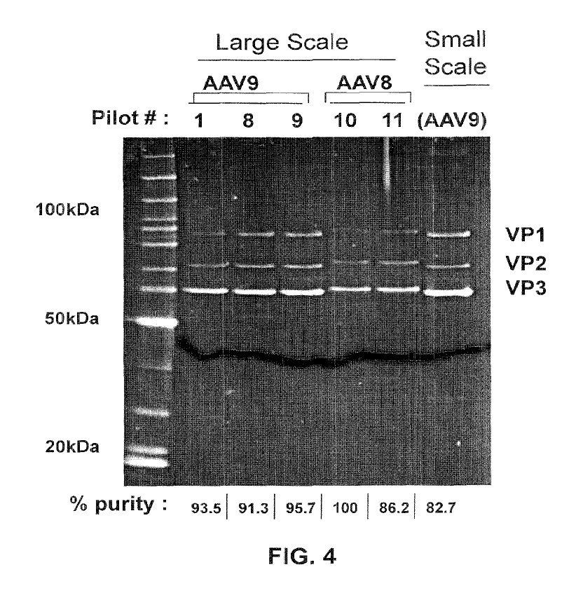

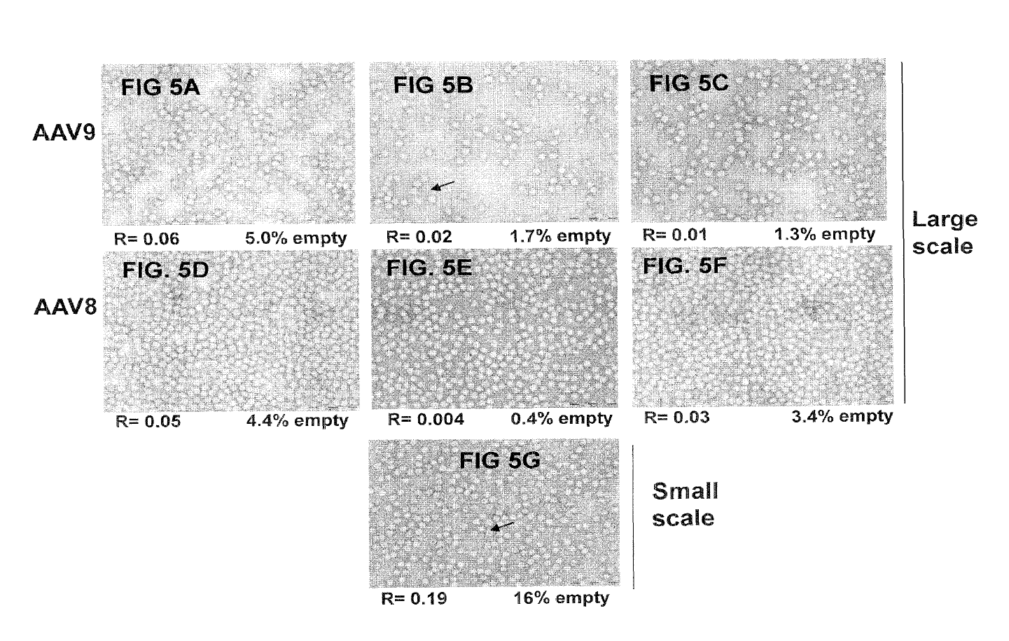

The vector lots produced in the pilot runs were characterized for capsid protein purity by SDS-PAGE analysis and for empty particle content by electron microscopy. Only a few minor bands in addition to the AAV capsid proteins VP1, 2 and 3 were visualized by SDS-PAGE analysis in each of the rAAV8 and rAAV9 large scale production lots, and the estimated purity exceeded 90% in all but a single case (FIG. 4). These results compared favorably with a standard small scale process, in which vector purities exceeding 85% are routinely achieved. Estimates of empty particle content of the large scale production lots were determined by direct observation of negatively stained vector particles on electron micrographs (FIGS. 5A-G). Empty particles are distinguished on these images by an electron-dense central region of the capsid in comparison to full particles, which exclude the negative stain. The empty particle content of the pilot production lots ranged from 0.4% to 5%. In unpurified preparations, the empty-to-full ratio can be as high as 30:1 (Sommer et al, 2003), and hence these results support the conclusion that iodixanol gradients are able to separate empty and full rAAV particles.

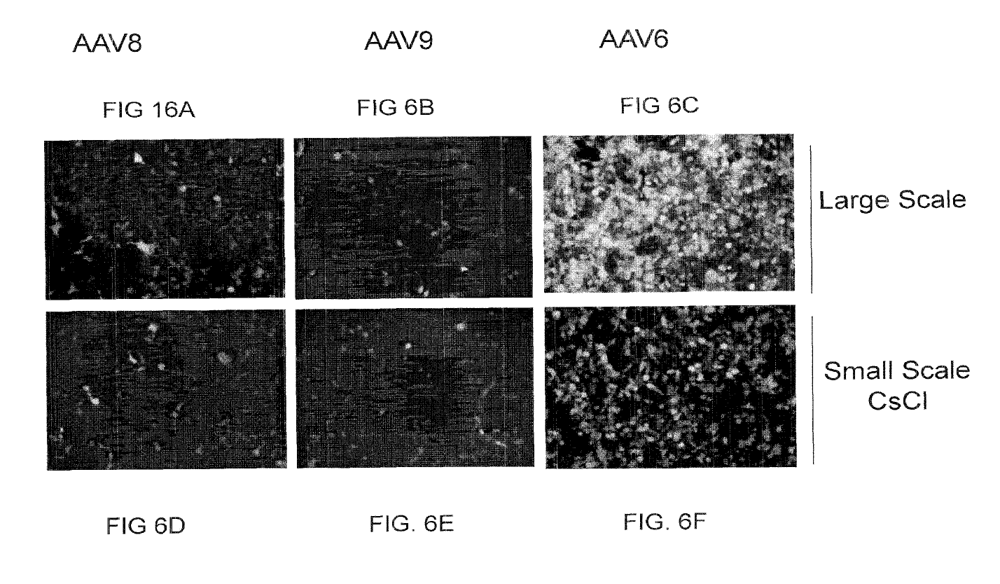

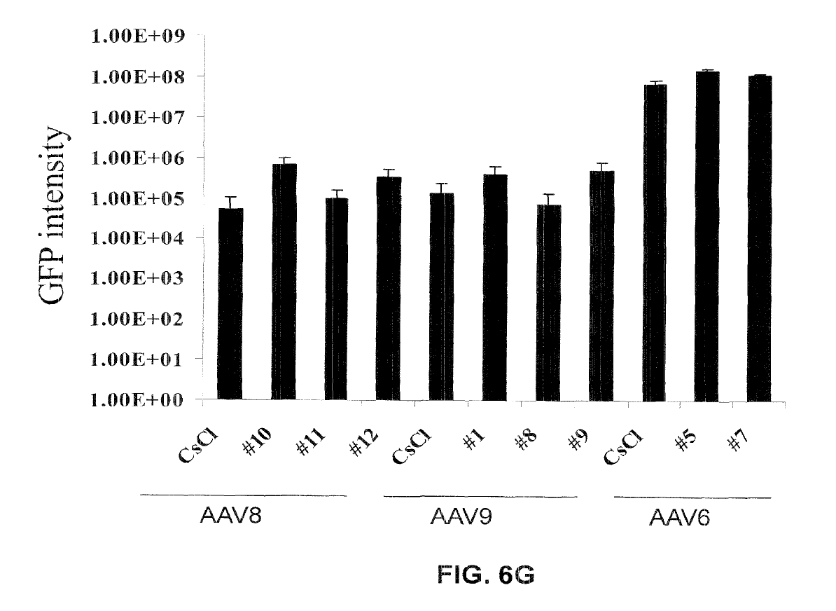

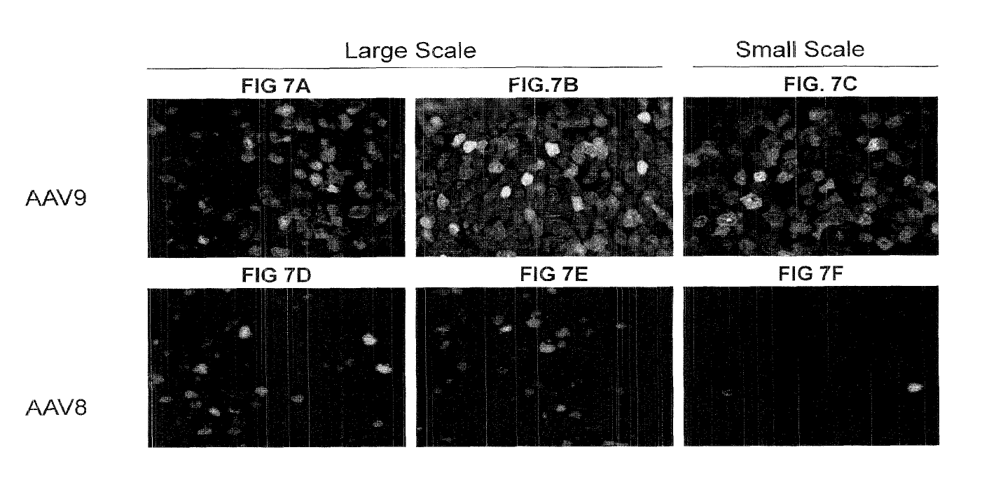

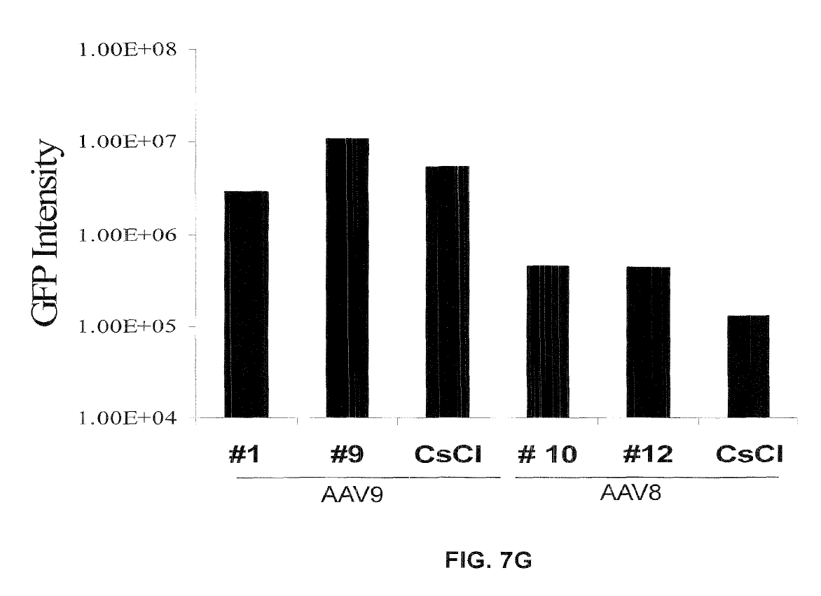

An essential quality of any rAAV production lot is the ability of the vector to deliver and express the gene of interest in cells. The potency of the rAAV8 and rAAV9 large scale production lots relative to vectors produced by a small scale process was assessed in vitro by eGFP expression and in C57BL16 mice livers of following IV injection (FIGS. 6A-G and FIGS. 7A-G, respectively). By both in vitro and in vivo analysis, all rAAV8 and rAAV9 vectors manufactured by the new production method exhibited equal or higher potency (up to 3.5-fold) when compared to identical vectors produced by the standard small scale approach. While rAAV6 vector yields were consistently low, the large scale production lots nonetheless exhibited a 2-fold transduction improvement compared to rAAV6-eGFP produced at small scale.

### 6.4. Discussion

The demand for rAAV vectors for clinical gene therapy continues to grow, and as the current progress in the field accelerates, large quantities of vector for use in late stage clinical trials may be needed. In parallel, a growing demand for vector to satisfy the complex requirements of pre-clinical studies is likely to rise as researchers rely increasingly on large animal data for improved prediction of clinical outcomes in humans. Several new processes for the production of rAAV vectors with yields sufficient to fuel late stage clinical trials are currently migrating from development labs to production suites both in industry and academic institutions (Clement et al. 2009; Virag et al. 2009; Zhang et al. 2009). However, these processes often involve time-consuming construction of intermediates such as hybrid viruses and packaging cell lines and are therefore ill-suited to the pre-clinical environment, where several combinations of transgenes and AAV serotypes must often be tested under strict timelines. Furthermore, the majority of pre-clinical work is performed in academic institutions where access to the high technology equipment used in many large scale production processes can be limited.

In order to support the vector requirements of a pre-clinical research group, a scaled production process was developed that would yield sufficient vector for large animal studies while retaining the flexibility and simplicity to rapidly generate any desired rAAV product in standard AAV laboratories. The production process described in this example is based upon PEI triple transfection, which allows retention of some unique properties of transfection-based production techniques, such as quick and easy substitutions of different AAV serotype/transgene combinations. A distinctive feature of the new process is that the majority of the vector can be harvested from the culture medium rather than from the production cells, and thus the bulk of cellular contaminants present in the cell lysate is avoided. The upstream process is extremely efficient and yields up to 2×10⁵ GC per cell, or 1×10¹⁵ GC per lot, of six cell stacks (FIG. 2; Table 3). The choice of iodixanol gradient centrifugation for the downstream process facilitates maintenance of a generic purification process for all serotypes. The isotonic, relatively inert nature of iodixanol has proven advantages with regard to maintaining vector potency (Zolotukhin et al. 1999) and overall product safety. By applying concentrated production culture medium to iodixanol step gradients, highly pure and potent rAAV vector was obtained with acceptable yield in a single one-hour centrifugation step. The whole process is rapid (7 days total, Table 4) and cost-effective. The average overall yields for AAV8 and AAV9 vectors were 2.2×10¹⁴ GC, with an overall process recovery of 26%.

In the current format, the production method is partially serum-free since the cells are grown in 10% fetal bovine serum prior to transfection. However, with animal product-free medium commercially available for 293 cells, the process can be adapted to be completely serum-free in compliance with safety regulations. Similarly, the process is cGMP compatible since all containers are sealed and manipulations are performed within the confines of a biosafety cabinet. Therefore, in addition for its utility for pre-clinical studies, the process is also adaptable for use in early stage clinical trials where vector demand is low, and for certain applications such as the treatment of inherited retinal diseases, where low vector doses are anticipated.

During development of the upstream process, rAAV of various serotypes was released to the supernatant in both calcium phosphate and PEI-transfected cultures (FIGS. 1A-D), and appears to occur in the absence of obvious cytopathology. The transfection technique used did not greatly influence the amount of vector released to the culture medium, but extending the incubation period post-transfection led to substantial increases in release. Moreover, when the medium was harvested and replaced on successive days post-transfection, the recovery of rAAV7 vector in the culture medium remained constant (data not shown). This observation suggests that the incorporation of perfusion culture techniques to the process may even further increase upstream yields. In the experiments reported in this example, adherent HEK 293 cell cultures were used for reasons of simplicity and convenience, but given the recently reported use of PEI transfection in the production of rAAV in suspension cultures (Durocher et al. 2007; Hildinger et al. 2007), this upstream process may also be adapted to bioreactors. An advantage of such an approach would be the ability to use the same upstream process for both pre-clinical and clinical vector manufacture, which is desirable from a regulatory standpoint.

The demonstration in this example that most AAV serotypes can be efficiently harvested from the production culture medium (FIG. 2; Vandenberghe et al, 2010) indicates that the new process will be widely applicable to most AAV vectors. However, for some AAV serotypes, modifications will be required. For example, rAAV2 is mostly retained in the cell (Vandenberghe et al. 2010), and release of this serotype to the culture medium would need to be optimized. rAAV6 vectors were not efficiently produced in either the cells or the culture medium following PE triple transfection (FIG. 2; Table 1), and was not reproducibly manufactured at high titer by standard calcium phosphate transfection and CsCl gradient purification.

Ion exchange, hydrophobic interaction or affinity column chromatography are the methods of choice for capture of AAV vector from large volumes of culture medium. These methods must often be developed specifically for each AAV serotype and, therefore, for pre-clinical vector production, a generic purification method to accommodate multiple serotypes is a better solution. The TFF concentration/iodixanol gradient method described in this example is a generic downstream approach to rAAV purification, and in the studies presented here produced a vector peak that was pure and relatively free of empty particles (FIG. 4 and FIGS. 5A-G). This example has formally demonstrated, for the first time, the ability of the iodixanol gradient purification method to separate empty from full rAAV particles.

The potency of the rAAV8 and rAAV9 vectors produced by the process described in this example was demonstrated herein in both in vitro and in vivo transduction assays to be at least equivalent, if not slightly better than, identical vectors produced by a routine small scale production method (FIGS. 6A-G and FIGS. 7A-G).

In conclusion, the large scale rAAV vector production process presented in this example is tailored toward the needs of AAV gene therapy laboratories involved in preclinical trials and is anticipated to satisfy most requirements of these studies, including the pre-clinical requirement for flexible vector manufacture. This AAV production process has the potential to be scaled up in order to supply rAAV vectors for clinical applications, while retaining the advantages of, e.g., reagent simplicity, process speed, and clearance of vector specific impurities.

### 6.5. References

Atkinson, M. A., Fung, V.P., Wilkins, P.C., Takeya, R., K, Reynolds, T.C., and Aranha, LL. 2005. Methods for generating high titer helper free preparations of release recombinant AAV vectors. US Published Patent Application No. 2005/0266567.
Auricchio, A., Hildinger, M., O'Connor, E., Gao, G.P., and Wilson, J. M. 2001, Isolation of highly infectious and pure adeno-associated virus type 2 vectors with a single step gravity-flow column. Hum Gene Ther 12(1): 71-76.
Brantly, M.L., Chulay, J. D., Wang, L., Mueller, C., Humphries, M., Spencer, L.T., Rouhani, F., Conlon, T. J., Calcedo, R., Betts, M. R. 2009. Sustained transgene expression despite T lymphocyte responses in a clinical trial of rAAV1-AAT gene therapy. Proc Natl Acad Sci USA 106(38): 16363-16368.
Brument, N., Morenweiser, R., Blouin, V., Toublanc, E., Raimbaud, I., Cherel, Y., Folliot, S., Gaden, F., Boulanger, P., Kroner-Lux, G. 2002. A versatile and scalable two-step ion-exchange chromatography process for the purification of recombinant adeno-associated virus serotypes-2 and -5. Mol Ther 6(5): 678-686.
Clement, N., Knop, D. R., and Byrne, B. J. 2009. Large-scale adeno-associated viral vector production using a herpesvirus-based system enables manufacturing for clinical studies. Hum Gene Ther 20(8): 796-806.
Davidoff, A. M., Ng, C. Y., Sleep, S., Gray, J., Azam, S., Zhao, Y., McIntosh, J. H., Karimipoor, M. and Nathwani, A.C. 2004. Purification of recombinant adeno-associated virus type 8 vectors by ion exchange chromatography generates clinical grade vector stock. J Virol Methods 121(2): 209-215.
Durocher, Y., Pham, P.L., St-Laurent, G., Jacob, D., Cass, B., Chahal, P., Lau, C. J., Nalbantoglu, J., and Kamen, A. 2007. Scalable serum-free production of recombinant adeno-associated virus type 2 by transfection of 293 suspension cells. J Virol Methods 144(1-2): 32-40.
Gao, G.P., Alvira, M. R., Wang, L., Calcedo, R., Johnston, J., and Wilson, J. M. 2002. Novel adeno-associated viruses from rhesus monkeys as vectors for human gene therapy. Proc Natl Acad Sci USA 99(18): 11854-11859.
Grimm, D., Kern, A., Rittner, K., and Kleinschmidt, I. A. 1998. Novel tools for production and purification of recombinant adeno-associated virus vectors. Hum Gene Ther 9(18): 2745-2760.
Hermens, W.T., Dijkhuizen, P. A., Sonnemans, M. A., Grimm, D., Kleinschmidt, J. A., and Verhaagen, J. 1999. Purification of recombinant adeno-associated virus by iodixanol gradient ultracentrifugation allows rapid and reproducible preparation of vector stocks for gene transfer in the nervous system. Hum Gene Ther 10(11): 1885-1891.
Hildinger, M., Baldi, L., Stettler, M., and Wurm, F. M. 2007. High-titer, serum free production of adeno-associated virus vectors by polyethyleneimine-mediated plasmid transfection in mammalian suspension cells. Biotechnol Lett 29(11): 1713-1721.
Kaludov, N., Handelman, B., and Chiorini, J. A. 2002. Scalable purification of adeno-associated virus type 2, 4, or 5 using ion-exchange chromatography. Hum Gene Ther 13(10): 1235-1243.
Maguire, A. M., Simonelli, F., Pierce, E. A., Pugh, E. N., Jr., Mingozzi, F., Bennicelli, J., Banfi, S., Marshall, K. A., Testa, F., Surace, E. M. 2008. Safety and efficacy of gene transfer for Leber's congenital amaurosis. N Engl J Med 358(21): 2240-2248.
Matsushita, T., Elliger, S., Elliger, C., Podsakoff, G., Villarreal, L., Kurtzman, G. J., Iwaki, Y., and Colosi, P. 1998. Adeno-associated virus vectors can be efficiently produced without helper virus. Gene Ther 5(7): 938-945.
Moss, RB., Rodman, D., Spencer, L.T., Aitken, M.L., Zeitlin, P.L., Waltz, D., MiHa, C., Brody, A.S., Clancy, J.P., Ramsey, B. 2004. Repeated adeno-associated virus serotype 2 aerosol-mediated cystic fibrosis transmembrane regulator gene transfer to the lungs of patients with cystic fibrosis: a multicenter, double-blind, placebo-controlled trial. Chest 125(2): 509-521.
Mueller, C. and Flotte, T. R 2008. Clinical gene therapy using recombinant adeno-associated virus vectors. Gene Ther 15(11): 858-863.
Neinhuis, A. 2009. Dose-Escalation Study Of A Self Complementary Adeno-Associated Viral Vector For Gene Transfer in Hemophilia B.
Okada, T., Nonaka-Sarukawa, M., Uchibori, R., Kinoshita, K., Hayashita-Kinoh, H., Nitahara-Kasahara, Y., Takeda, S., and Ozawa, K. 2009. Scalable purification of adeno-associated virus serotype 1 (AAV1) and AAV8 vectors, using dual ion-exchange adsorptive membranes. Hum Gene Ther 20(9): 1013-1021.
Qu, G., Bahr-Davidson, J., Prado, J., Tai, A., Cataniag, F., McDonnell, J., Zhou, J., Hauck, B., Luna, J., Sommer, J. M. 2007. Separation of adeno-associated virus type 2 empty particles from genome containing vectors by anion-exchange column chromatography. J Virol Methods 140(1-2): 183-192.
Salvetti, A., Oreve, S., Chadeuf, G., Favre, D., Cherel, Y., Champion-Arnaud, P., David-Ameline, J., and Moullier, P. 1998. Factors influencing recombinant adeno-associated virus production. Hum Gene Ther 9(5): 695-706.
Schroder, M., Schafer, R, and Friedl, P. 1997. Spectrophotometric determination of iodixanol in subcellular fractions of mammalian cells. Anal Biochem 244(1): 174-176.
Smith, R. H., Levy, J. R., and Kotin, R. M. 2009. A simplified baculovirus-AAV expression vector system coupled with one-step affinity purification yields high-titer rAAV stocks from insect cells. Mol Ther 17(11): 1888-1896,
Snyder, RO. and Flotte, T. R 2002. Production of clinical-grade recombinant adeno-associated virus vectors. Curr Opin Biotechnol 13(5): 418-423.
Sommer, J. M., Smith, P. H., Parthasarathy, S., Isaacs, J., Vijay, S., Kieran, J., Powell, S. K., McClelland, A., and Wright, J.F. 2003. Quantification of adeno-associated virus particles and empty capsids by optical density measurement. Mol Ther 7(1): 122-128.
Vandenberghe, L. H., Lock, M., Xiao, R., Lin, J., Korn, M., and Wilson, J. M. 2010. Heparin-dependent release of AAV into the supernatant simplifies manufacturing. Submitted, and now published as “Efficient serotype-dependent Release of Functional Vector into the Culture Medium During Adeno-Associated Virus Manufacturing”, Hu Gene Ther, 21:1251-1257 (October 2010).
Virag, T., Cecchini, S., and Kotin, R. M. 2009. Producing recombinant adeno-associated virus in foster cells: overcoming production limitations using a baculovirusinsect cell expression strategy. Hum Gene Ther 20(8): 807-817.
Wang, L., Wang, H., Bell, P., McCarter, R. J., He, I, Calcedo, R., Vandenberghe, L. H., Morizono, H., Batshaw, M.L., and Wilson, J. M. 2010. Systematic evaluation of AAV vectors for liver directed gene transfer in murine models. Mol Ther 18(1): 118-125.
Warrington, K. H., Jr. and Herzog, R. W. 2006. Treatment of human disease by adeno-associated viral gene transfer. Hum Genet 119(6): 571-603.
Wright, J.F. 2009. Transient transfection methods for clinical adeno-associated viral vector production. Hum Gene Ther 20(7): 698-706.
Wright, J.F., Le, T., Prado, J., Bahr-Davidson, J., Smith, P. H., Zhen, Z., Sommer, J. M., Pierce, G.F., and Qu, G. 2005. Identification of factors that contribute to recombinant AAV2 particle aggregation and methods to prevent its occurrence during vector purification and formulation. Mol Ther 12(1): 171-178.
Xiao, X., Li, J., and Samulski, R. J. 1998. Production of high-titer recombinant adeno-associated virus vectors in the absence of helper adenovirus. 3 Virol 72(3): 2224-2232.
Zhang, H., Xie, J., Xie, Q., Wilson, J. M., and Gao, G. 2009. Adenovirus-adeno-associated virus hybrid for large-scale recombinant adeno-associated virus production. Hum Gene Ther 20(9): 922-929.
Zolotukhin, S., Byrne, B. J. Mason, E., Zolotukhin, 1., Potter, M., Chesnut, K., Summerford, C., Samulski, R. J., and Muzyczka, N. 1999. Recombinant adeno-associated virus purification using novel methods improves infectious titer and yield. Gene Ther 6(6): 973-985.
Zolotukhin, S., Potter, M., Zolotukhin, I., Sakai, Y., Loiler, S., Fraites, T. J., Jr., Chiodo, V. A., Phillipsberg, T., Muzyczka, N., Hauswirth, W. W. 2002. Production and purification of serotype 1, 2, and 5 recombinant adeno-associated viral vectors. Methods 28(2): 158-167.

### 7. EXAMPLE 2

### Cesium Purification of AAV Vectors

This example describes a new procedure for cesium chloride (CsCl) purification of AAV vectors from transfected cell pellets.

### Day 1—Pellet Processing and CsCl Spin

1) Lysate Preparation
Thaw cells from −80° C. freezer for 15 minutes at 37° C.
Resuspend the cell pellet in ^{˜}20 mL of Resuspension Buffer 1(50 mM Tris, pH 8.0, 2 mM MgCl) for 40 plates of cells and for a final volume of 20 mL, and place on ice.
Freeze/thaw 3 times (dry ice and ethanol bath/37° C. water bath).
Add 100 μL of Benzonase (250 U/mL) per prep and invert gently, incubate the samples at 37° C. for 20 minutes, inverting the tube every 5 min.
Add 6 mL of 5M NaCl to bring the final salt concentration to 1 M. Mix.
Spin at 8,000 rpm for 15 min at 4° C. in Sorval centrifuge. Note: Ensure the Sorval is clean. After centrifugation, sterilize tube with 70% before proceeding further. Transfer supernatant to a new tube,
Spin again at 8,000 rpm for 15 min at 4° C. in Sorval. Note: Ensure the Sorval is clean. After centrifugation, sterilize tube with 70% before proceeding further.
Add 1.8 mL of 10% OGP for a final concentration of 0.5%, and mix gently by inversion.

2) Cesium Chloride Step Gradient Purification
For each preparation, prepare two 2-tier gradients consisting of 7.5 mL of 1.5 g/mL CsCl and 15 mL 1.3 g/mL CsCl in Beckman SW-28 tubes (do not use ultraclear tubes). Load the less dense CsCl first and then bottom load the heavier CsCl.
Add 15 mL of sample to the top of each gradient. Add sample slowly to the side of the tube so as not to disturb the gradient. Label the tubes with lot #.
Spin at 25,000 rpm at 15° C. for 20 hours minimum.
   Day 2—Collect AAV Band from 1st CsCl Spin and Set Up 2nd CsCl Spin

1) Collect Band from CsCl Spin
Carefully remove the centrifuge tubes (A & B) out of the bucket, taking care not to disturb the gradient. Secure the first tube (A) on a tube holder.
Take a pre-sterilized 2 ft length of tygon-silicone tubing (1.6 mm inner diameter; Fisher NC9422080) fitted with two 1/16 inch male luers (Fisher NC9507090) and insert 18 G 1″ needles into the luers.
Pierce the tube at a right angle as close to the bottom as possible with one of the 18 G 1″ needles (bevel facing up), and clamp the tubing into the easy load rollers of the masterflex pump. Gently increase the speed to ^{˜}1 mL/min. Collect the first 4.5 mL into a 15 mL falcon tube and then start to collect fractions (250 μL) into a 96 well plate (from tube A). Collect 48 fractions.
Run the rest of the gradient into a beaker containing a 20% bleach solution and discard the needle/tubing assembly.
Take another pre-sterilized 2 ft length of tygon-silicone tubing (1.6 mm inner diameter; Fisher NC9422080) fitted with two 1/16 inch male liters (Fisher NC9507090) and insert 18 G 1″ needles into the luers for collecting fractions from second tube (from tube B).
Repeat the entire harvest for the tube B. Discard the needle/tubing assembly after use.

2) Read Refraction Index (RJ)
Using a multichannel pipetter, transfer 10 μL of each fraction (of the 48 collected, first from 96-well plate A) to a fresh plate (label with 1 to 48) and leave the remainder of the fractions in the biosafety cabinet.
Take 5 μL of each fraction and read the RI using a refractometer. The fractions containing AAV should have a refractive index of 1.3740-1.3660. Read the RI down to 1.3650 and then pool the fractions in the biosafety cabinet with RI in the 1.3740 to 1.3660 range. (Measure the total volume after pooling both the 96-well plates belonging to tube 1 and 2. In case there is still some space for adding more, add from wells with RI of 1.375.)
Repeat this process for the second 96-well plate (from tube B).

3) Load the Second Gradient
The total pooled volume from each gradient (from tubes A and B) should be 5-6 mL. Pool the two gradient harvests in a 50 mL falcon tube and bring the volume to 13 mL with a 1.41 g/mL solution of CsCl. Mix well with a pipette.
Using a 10 mL syringe and 18 G needle, add the pooled first gradient harvest to a 13 mL sealable centrifuge tube. The solution should be added to the line on the neck of the tube with no bubbles.
Seal the tube using the portable sealer, metal tube caps and heat sink.
Squeeze the tube to test for leaks and then place in a Ti70.1 rotor with the appropriate balance. Insert the rotor caps and lid and then spin at 60,000 rpm, 15° C. for 20 hours.
   Day 3—Collect AAV Band from 2nd CsCl Spin and Desalt

1) Collect Band from CsCl Spin

Carefully remove the centrifuge tube out of the bucket, taking care not to disturb the gradient. Secure the tube on a tube holder. At this point a single band should be visible after bottom illumination about halfway up the tube.
Take a pre-sterilized 2 ft length of tygon-silicone tubing (1.6 mm inner diameter; Fisher NC9422080) fitted with two 1/16 inch male luers (Fisher NC9507090) and insert 18 G 1″ needles into the luers. Use 1 length of tubing per prep.
Pierce the tube at a right angle as close to the bottom as possible with one of the 18 G 1″ needles (bevel facing up) and clamp the tubing into the easy load rollers of the masterflex pump. Pierce the tube again at the top with a second 18 G needle. Gently increase the speed to ^{˜}1 mL/min and then start to collect fractions (250 μL) into a 96 well plate. Collect the whole gradient (−45 fractions).

2) Read Refractive Index (RI):
Using a multichannel pipetter, transfer 10 μl of each fraction to a fresh plate and leave the remainder of the fractions in the biosafety cabinet.
Take 5 μL of each fraction and read the RI using a refractometer. The fractions containing AAV should have a refractive index of 1.37504.3660. Read the RI down to 1.3650, and then pool fractions with RI in range of 1.3750 to 1.3660.

3) Desalting: Amicon Ultra-I 5 Centrifugal Concentrators

In this procedure the vector is diluted with PBS and spun at low speed through the 100 kDa MWCO filter device. Because of the large molecular weight of AAV Particles (˜5000 kDa), the vector is retained by the membrane and the salt passes through. Vector can build up on the membranes, so rinsing is required at the final stage.
Aliquot 50 mL PBS+35 mM NaCl into a 50 mL tube.
Dilute the pooled fractions from step 2 above with the PBS+35 mM NaCl to 15 mL total volume. Mix gently and add to Amicon filter device.
Spin in a bench top Sorvall centrifuge at 2,000 to 4,000 rpm for 2 minutes. Because it is important to keep the level of the liquid above the top of the filter surface (−1.8 mL) at all times so that the vector does not dry onto the membrane, it is recommended that the lower speed spin is attempted first to determine the flow rate of the sample. The goal is to reduce the volume of the retentate to ˜1.8 mL. An additional short spines) may be necessary to achieve this. If the volume does go below that desired, bring it back to 1.8 mL with PBS+35 mM NaCl.
Add a further 13.2 mL PBS+35 mM NaCl, mix by pipette with the retentate remaining in the device, and repeat the spinning process described above. Continue this process until all the 50 mL PBS+35 mM NaCl aliquoted previously is spun through the device.
Rinse the membrane with the final retentate (˜1.8 mL) by repeatedly pipetting against the entire surface. Recover the retentate into a suitably-sized sterile centrifuge tube using 1 mL and 200 μL Eppendorf tips (the 200 μL tip is for the final retentate at the bottom of the device that is inaccessible to a 1 mL tip). Rinse the membrane twice using a minimum of 100 μL of PBS+35 mM NaCl and pool it with your final retentate.
Determine the exact volume and add glycerol to 5%.

Aliquot into 5×25μ: aliquots, 1×100 μL for archive, and the rest into 105 μL aliquots.
Freeze immediately at −80° C.
   Reagents Used in rAAV Purification
Resuspension buffer 1 [50 mM Tris (pH 8.0), 2 mM MgCl]: 50 mL 1 M Tris (pH 8.0), 2 mL/M MgCh to 948 mL MQ water, filter sterilize.
1.5 g/mL CsCl solutions: dissolve 675 g of CsCl in 650 mL PBS and adjust final volume to 1000 mL. Weigh 1 mL of the solution to check the density. Filter sterilize the solution.
1.3 g/mL CsCl solutions: dissolve 405 g of CsCl in 906 mL PBS and adjust final volume to 1000 mL. Weigh 1 mL of the solution to check the density. Filter sterilize the solution.
10% (W/V) Octyl-PD-glucopyranoside (OGP) (Sigma, 08001-10G): Bring 10 grams to 100 mL with milliQ water. Filter sterilize the solution.
Final formulation buffer: PBS+35 mM NaCl. To 1 liter sterile PBS, add 7.05 mL sterile 5 M NaCl.
Sterile glycerol: Aliquot glycerol into 100 mL glass bottles. Autoclave for 20 minutes on liquid cycle.

### 8. EXAMPLE 3

### DNA Constructs for Preparation of PITA AAV Vectors

The invention is illustrated by Examples 3-5, which demonstrate the tight regulation of ablator expression using rapamycin, to dimerize transcription factor domains that induce expression of Cre recombinase; and the successful inducible ablation of a transgene containing Cre recognition sites (loxP) in cells. The tight regulation of expression of the ablator is demonstrated in animal models.

The following are examples of DNA constructs DNA constructs and their use to generate replication-defective AAV vectors for use in accordance with the PITA system of the invention is illustrated in the examples below.

### 8.1. Constructs Encoding a Dimerizable Transcription Factor Domain Unit and an Ablation Unit

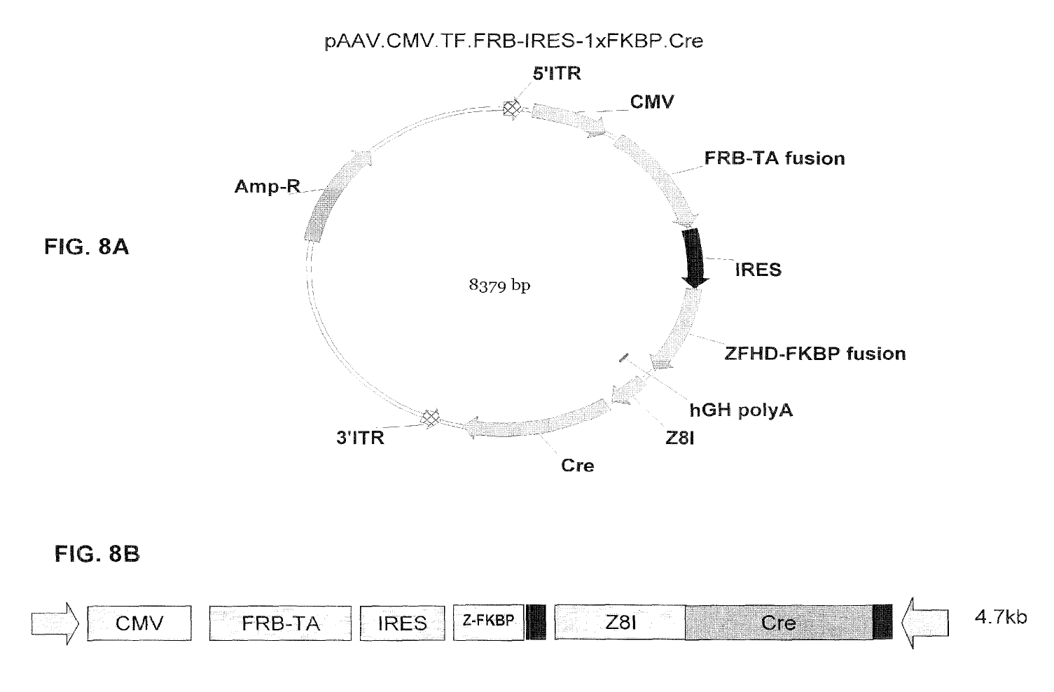

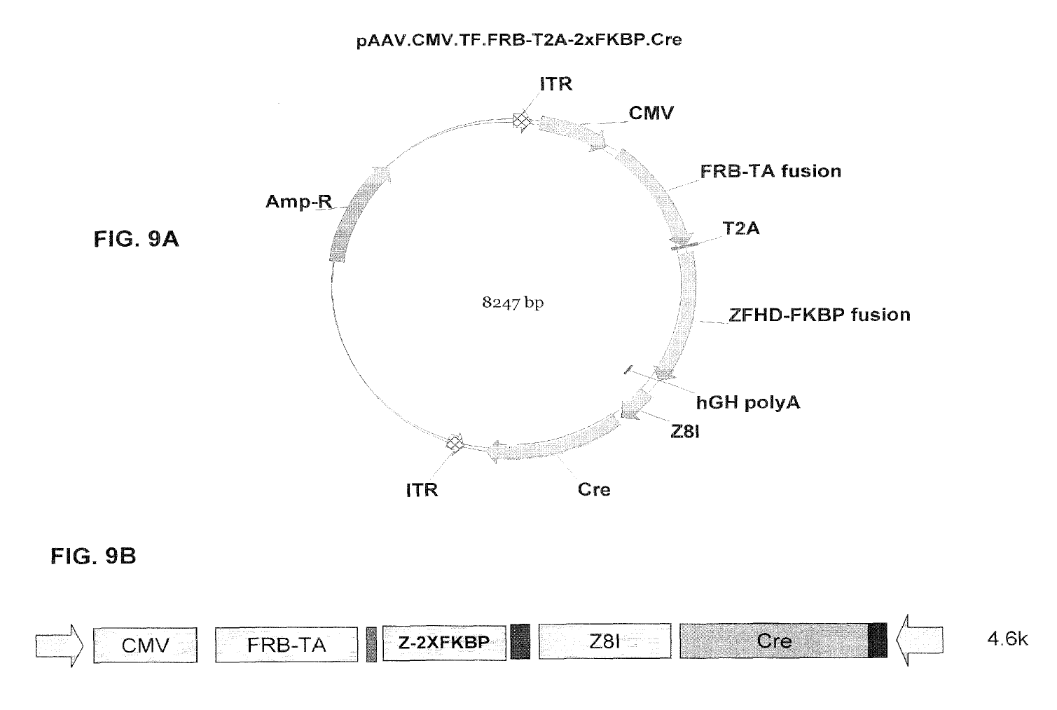

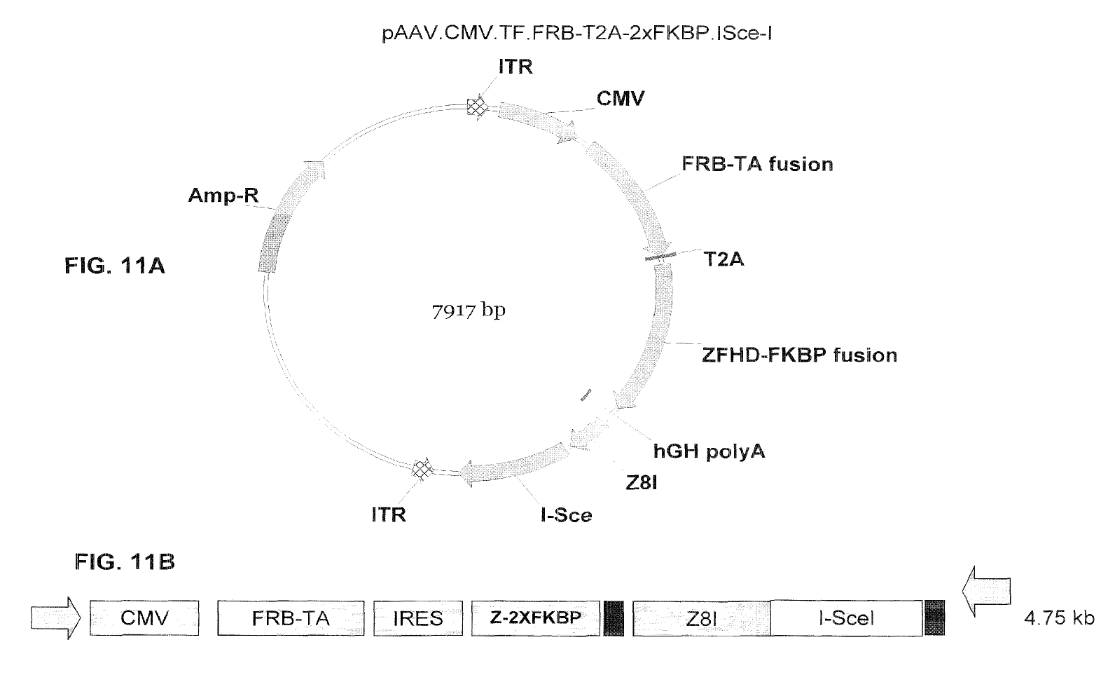

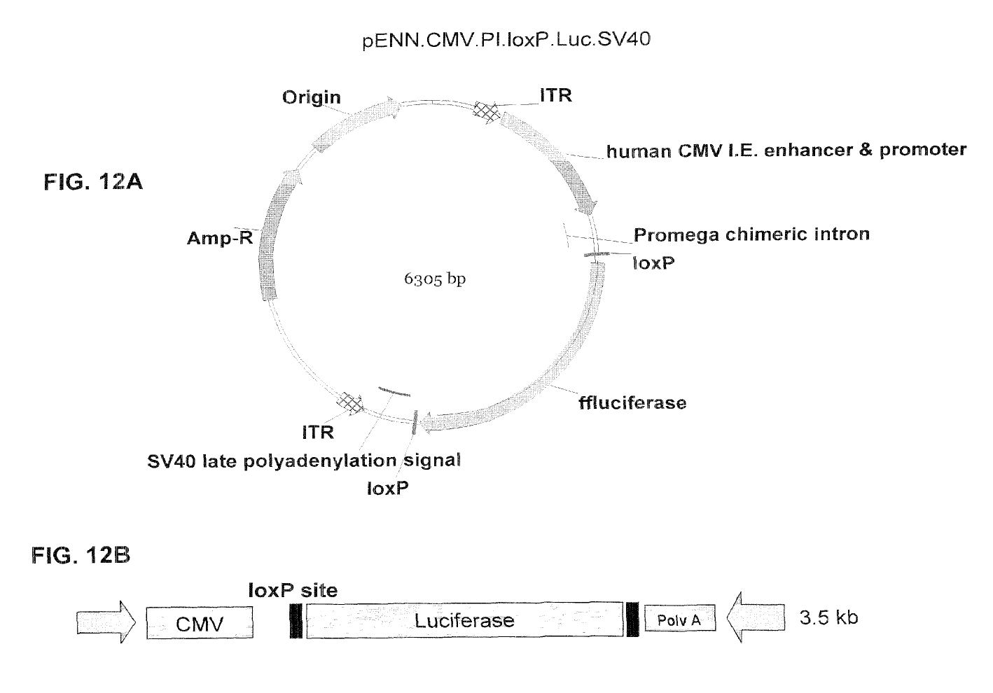

FIGS. 8A-B through FIG. 12B are diagrams of the following DNA constructs that can be used to generate AAV vectors that encode a dimerizable transcription factor domain unit and an ablation unit: (1) pAAV.CMV.TF.FRB-TIRES-1xFKBP.Cre (FIGS. 8A-B); (2) pAAV.CMV.TF.FRB-T2A-2xFKBP.Cre (FIGS. 9A-B); (3) pAAV.CMVI73.TF.FRB-T2A-3xFKBP.Cre (FIGS. 10A-B); and (4) pAAV.CMV.TF.FRB-T2A-2xFKBP.ISce-I (FIGS. 11A-B).

A description of the various domains contained in the DNA constructs follows:

ITR: inverted terminal repeats of AAV serotype 2 (168 bp). [SEQ ID NO: 26]
CMV: full cytomegalovirus (CMV) promoter; including enhancer. [SEQ ID NO 27]
CMV (173 bp): minimal CMV promoter, not including enhancer. [SEQ ID NO: 28]
FRB-TA fusion: fusion of dimerizer binding domain and an activation
domain of a transcription factor (900 bp, SEQ ID NO: 29). The protein is provided herein as SEQ ID NO: 30. The FRB fragment corresponds to amino acids 2021-2113 of FRAP (FKBP rapamycin-associated protein, also known as mTOR [mammalian target of rapamycin]), a phosphoinositide 3-kinase homolog that controls cell growth and division. The FRAP sequence incorporates the single point-mutation Thr2098Leu (FRAP_{L}) to allow use of certain non-immunosuppressive rapamycin analogs (rapalogs). FRAP binds to rapamycin (or its analogs) and FKBP and is fused to a portion of human NF-KB p65 (190 amino acids) as transcription activator.
ZFHD-FKBP fusion: fusion of a DNA binding domain and 1 copy of a Dimerizer binding domain (1xFKBP; 732 bp), 2 copies of drug binding domain (2xFKBP;
1059 bp), or 3 (3xFKBP; 1389 bp) copies of drug binding domain. Immunophilin FKBP (FK506-binding protein) is an abundant 12 kDa cytoplasmic protein that acts as the intracellular receptor for the immunosuppressive drugs FK506 and rapamycin. ZFHD is DNA binding domains composed of a zinc finger pair and a homeodomain. Both fusion proteins contain N-terminal nuclear localization sequence from human c-Myc at the 5′ end. See, SEQ ID NO: 45.
T2A: self cleavage peptide 2A (54 bp) (SEQ ID NO: 31).
Z8I: 8 copies of the binding site for ZFHD (Z8) followed by minimal promoter from the human interleukin-2 (IL-2) gene (SEQ ID NO 32). Variants of this promoter may be used, e.g., which contain from 1 to about 20 copies of the binding site for ZFHD followed by a promoter, e.g., the minimal promoter from IL-2.
Cre: Cre recombinase. Cre is a type I topoisomerase isolated from bacteriophage P1. Cre mediates site specific recombination in DNA between two loxP sites leading to deletion or gene conversion (1029 bp, SEQ ID NO: 33).
I-SceI: a member of intron endonuclease or homing endonuclease which is a large class of meganuclease (708 bp, SEQ ID NO: 34). They are encoded by mobile genetic elements such as introns found in bacteria and plants. I-SceI is a yeast endonuclease involved in an intron homing process. I-SceI recognizes a specific asymmetric 18 bp element, a rare sequence in mammalian genome, and creates double strand breaks. See, Jasin, M. (1996) Trends Genet., 12, 224-228.
hGH poly A: minimal poly adenylation signal from human GH (SEQ ID NO: 35).
IRES: internal ribosome entry site sequence from ECMV (encephalomyocarditis virus) (SEQ ID NO: 36).

### 8.2. Constructs Encoding Transgene Units

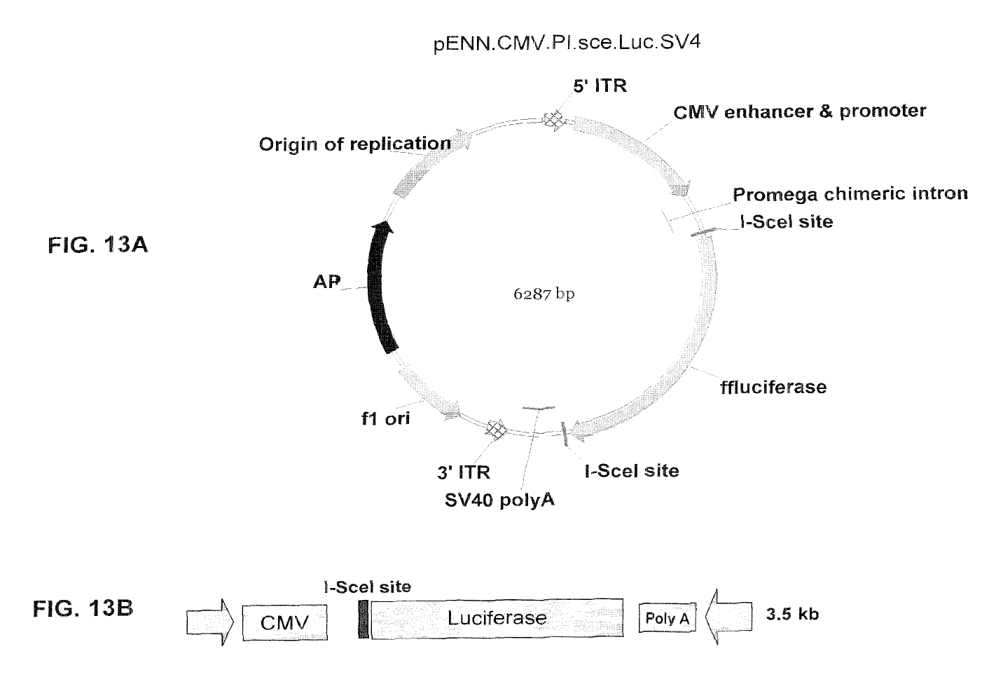

FIGS. 12A-B and FIGS. 13A-B are diagrams of the following DNA constructs for generating an AAV vector encoding a transgene flanked by loxP recognition sites for Cre recombinase:

(1) pENN.CMV.PI.loxP.Luc.SV40 (FIGS. 12A-B); and (2)

pENN.CMV.PI.sce.Luc.SV40 (FIGS. 13A-B). A description of the various domains of the constructs follows:

ITR: inverted terminal repeats of AAV serotype 2 (SEQ ID NO: 26).

CMV: cytomegalovirus (CMV) promoter and enhancer regulating immediate early genes expression (832 bp, SEQ ID NO: 27).

loxP: recognition sequences of Cre. It is a 34 bp element comprising of two 13 bp inverted repeat flanking an 8 bp region which confers orientation (34 bp, SEQ ID NO: 37).

Ffluciferase: fire fly luciferase (1656 bp, SEQ ED NO: 38).

SV 40: late polyadenylation signal (239 bp, SEQ ID NO: 39).

I-SceI site: SceI recognition site (18 bp, SEQ ID NO: 25).

### 8.3. Constructs Encoding a Transgene Unit and a Dimerizable Transcription Factor Domain Unit

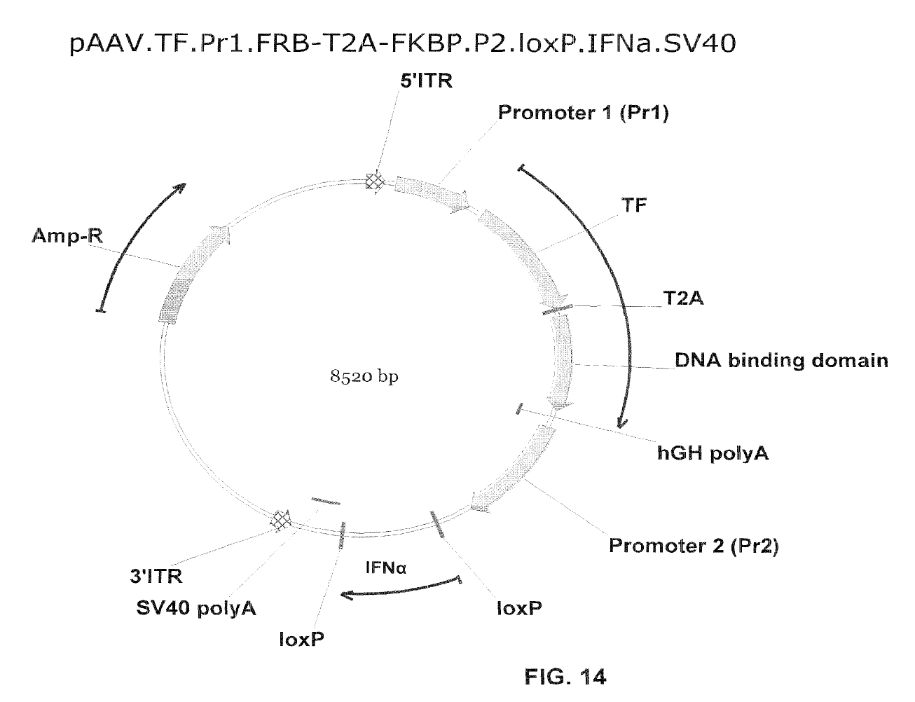

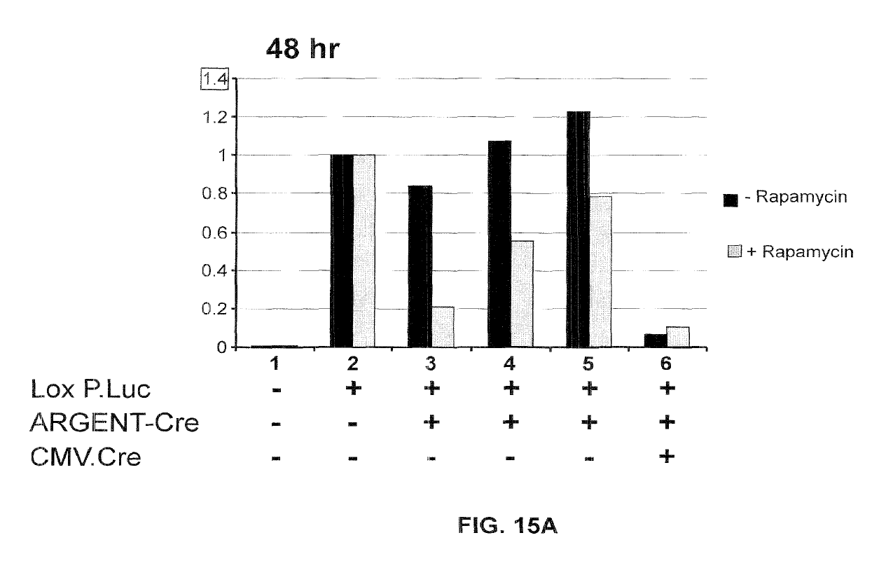

FIG. 14 is a diagram of DNA construct for generating an AAV vector that contains a transgene unit and a dimerizable transcription factor domain unit. This plasmid provides, on AAV plasmid backbone containing an ampicillin resistance gene, an AAV 5′ ITR, a transcription factor (TF) domain unit, a CMV promoter, an FRB (amino acids 2021-2113 of FRAP (FKBP rapamycin-associated protein, also known as mTOR [mammalian target of rapamycin]), a phosphoinositide 3-kinase homolog that controls cell growth and division), a T2A self-cleavage domain, an FKBP domain, and a human growth hormone polyA site, a CMV promoter, a loxP site, an interferon alpha coding sequence, and an SV40 polyA site. The ablation unit (cre expression cassette) can be located on a separate construct. This strategy could minimize any potential background level expression of cre derived from upstream CMV promoter.

### 9. EXAMPLE 4

### In Vitro Model for PITA

This example demonstrates that the DNA elements (units) engineered into the AAV vectors successfully achieve tightly controlled inducible ablation of the transgene in cells. In particular, this example shows that luciferase transgene expression can be ablated upon dimerizer (rapamycin) treatment of cells transfected with constructs containing a transgene unit (expressing luciferase and containing lox p sites), an ablation unit (expressing Cre), and a dimerizable transcription factor domain unit.

Human embryonic kidney fibroblast 293 cells were seeded onto 12 well plates. Transfection of the cells with various DNA constructs described in section 9.1 herein was carried out the next day when the cell density reached 90% confluency using lipofectamine 2000 purchased from Invitrogen. A vector encoding enhanced green fluorescent protein (EGFP) was added at 10% of total DNA in each well to serve as internal control for transfection. The DNA suspended in DMEM was mixed with lipofectamine 2000 to form DNA-lipid complex and added to 293 cells for transfection following instructions provided by Invitrogen Corporation. At 6 hours post transfection, half of the wells were treated with rapamycin at a final concentration of 50 nM. Culture medium (DMEM supplemented with 10% FBS) was replaced daily with fresh rapamycin. At 48 and 72 hour post transfection, cells were washed once with PBS and then scraped out of the well, resuspended in lysis buffer supplied in Luciferase assay kit purchased from Promega. The cell suspension was vortexed and the debri spun down. The luciferase activity was determined by mixing 10 μL of the lysate with 100 μL of the substrate and light emission per second read from a luminometer.

### 9.1. Constructs

The following constructs, most of which are described in Section 8, Example 3, were used to generate infectious, replication-defective AAV vectors:

1. pENN.AAV.CMV.RBG as a control, containing a CMV promoter and no transgene
2. pENN.CMV.PI.loxP.Luc.SV40 (FIGS. 12A-B)/pENN.AAV.CMV.RBG (CMV promoter and no transgene)
3. pENN.CMV.PI.loxP.Luc.SV40(FIGS. 12A-B/pAAV.TF.CMV.FRB-T2A-2xFKBP.Cre (FIGS. 9A-B)
4, pENN.CMV.PI.loxP.Luc.SV40(FIGS. 12A-B/pAAV.TF.CMV.FRB-IRES-FKBP.Cre (FIGS. 8A-B)
5. pENN.CMV.PI.loxP.Luc.SV40(FIGS. 12A-B)/pAAV.CMVI73.FRB-T2A-3xFKBP.Cre (FIGS. 10A-B)
6. pENN.CMV.PI.loxP.Luc.SV40(FIGS. 12A-B)/pENN.AAV.CMV.PI.Cre.RBG, which expresses the Cre gene from a constitutive promoter

### 9.2. Results

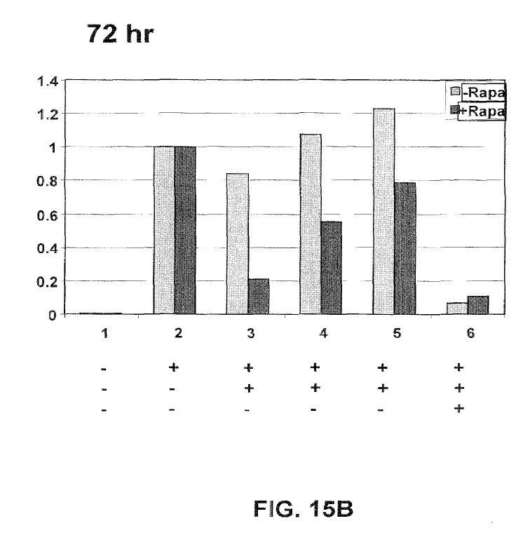

The results at 48 hours are shown in FIG. 15A and the results at 72 hours are shown in FIG. 15B. In the control (treatment 6), where Cre is constitutively expressed, luciferase expression was ablated independently of rapamycin compared to the control expression of luciferase without 10xP sites (treatment 2, cells transfected with luciferase construct). In contrast, in cells receiving the 10xP flanking luciferase construct plus one of the constructs carrying cre under the control of PITA system (treatment 3, 4 and 5), the level of the reporter gene expression is comparable to the control in the absence of dimerizer, rapamycin, indicating very little or no cre expression is induced. However, upon induction by treatment with rapamycin, the level of reporter gene expression in cells receiving PIT A controlled cre constructs were significantly reduced compared to the control (treatment 2), indicating cre expression was activated. The results confirm that the expression of the ablator is specifically regulated by the dimerizer, rapamycin.

### 10. EXAMPLE 5

### In Vivo Model for a Dimerizer-Inducible System

This example shows tight tissue-specific control of transgene expression using a liver-specific promoter that is regulated by the dimerizer-inducible system described herein. These data serves as a model for tight regulation of the ablator in the PITA system.

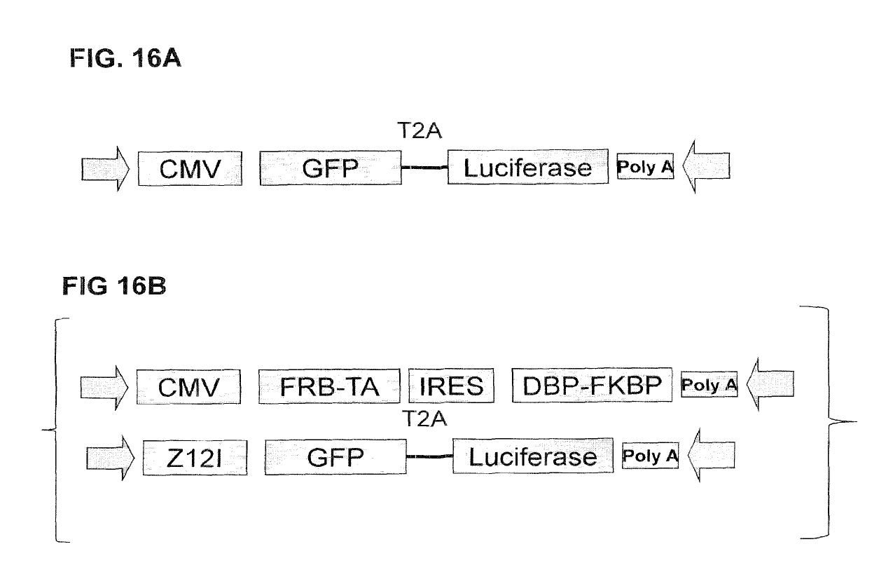

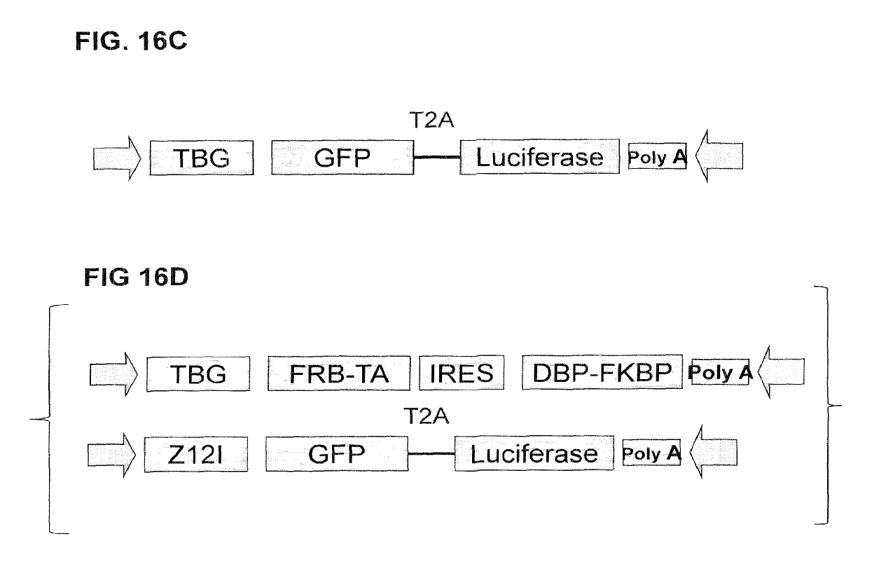

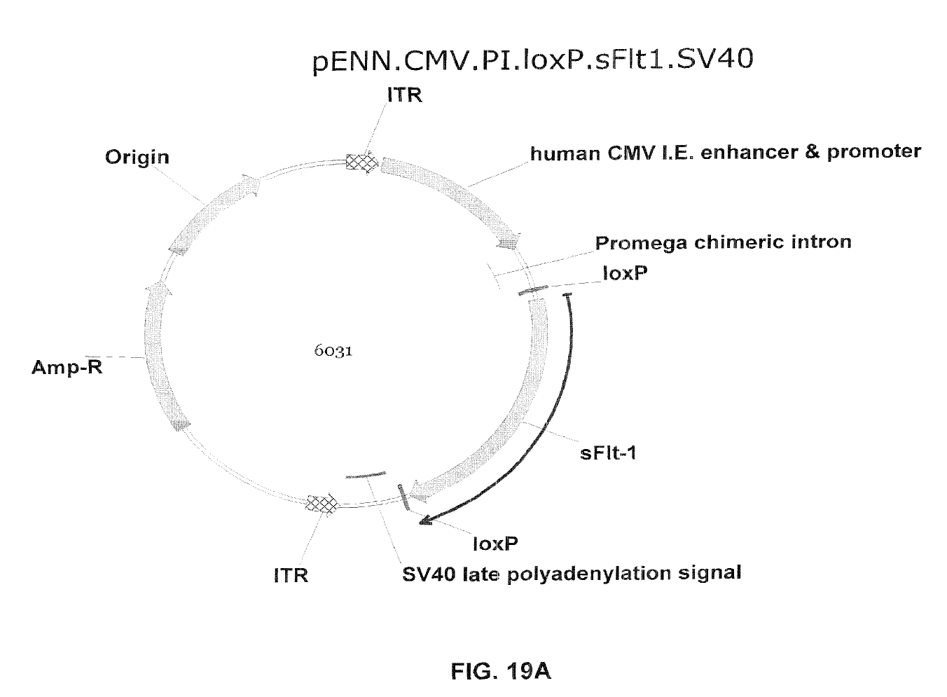

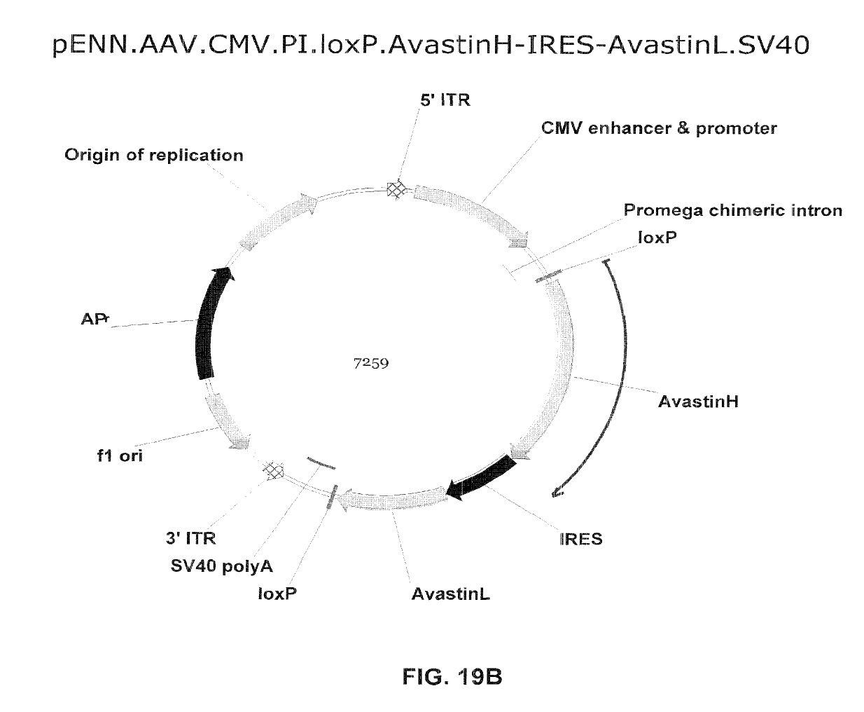

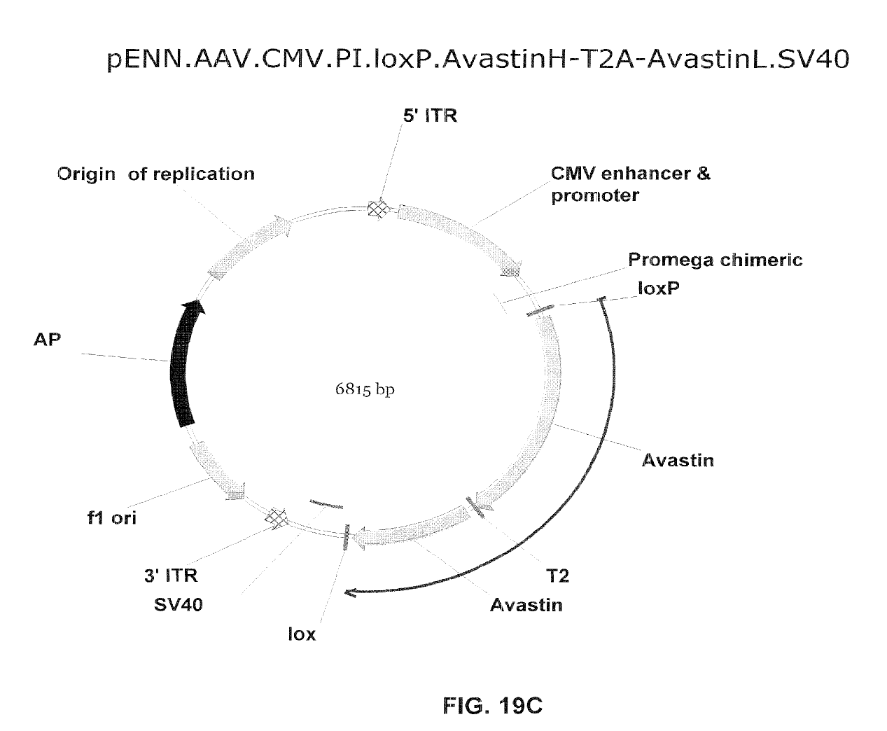

Four groups of three mice received IV injection of AAV vectors encoding bicistronic reporter genes (GFP-Luciferase) at doses of 3×10¹⁰, 1×10¹¹ and 3×10¹¹ particles of virus, respectively: Group 1 (G 1, G2, and G3) received AAV vectors expressing GFP Luciferase under the control of ubiquitous constitutive CMV promoter (see FIG. 16A for a diagram of the DNA construct). Group 2 (G4, G5, and G6) received co-injection of the following 2 AAV vectors: (1) AAV vector expressing a dimerizable transcription factor domain unit (FRB fused with p65 activation domain and DNA binding domain ZFHD fused with 3 copies of FKBP) driven by the CMV promoter (the DNA construct shown in FIG. 9B; and (2) AAV vector expressing GFP-Luciferase driven by a promoter induced by the dimerized TF (see FIG. 19C for a diagram of the DNA constructs). Group 3 (G7, G8, and G9) received AAV vector expressing GFP-Luciferase under the control of a liver constitutive promoter, TBG (see FIG. 16C for a diagram of the DNA construct). Group 4 (G10, G11, G12) received co-injection of the following 2 AAV vectors: (1) AAV vector expressing a dimerizable transcription factor domain unit (FRB fused with p65 activation domain and DNA binding domain ZFHD fused with 3 copies of FKBP) driven by the TBG promoter; and (2) AAV vector expressing GFP-Luciferase driven by a promoter induced by the dimerized TF (see FIG. 16D for a diagram of the DNA constructs).

About 2 weeks post virus administration, the mice were given IP injection of the dimerizer, rapamycin, at the dose of 2 mg/kg. Starting the next day the luciferase expression was monitored by Xenogen imaging analysis. Approximately 24 hours post rapamycin injection, the mice were IP injected with luciferin, the substrate for luciferase, then anesthetized for imaging.

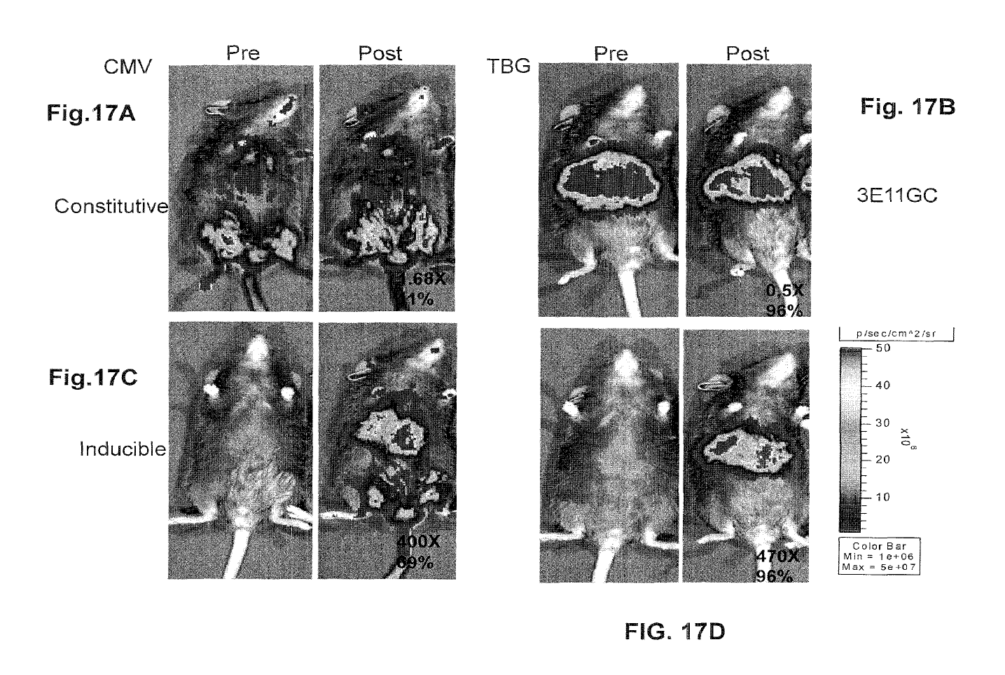

The mice that received 3×10¹¹ particles of virus had images taken 30 min post luciferin injection (FIGS. 17A-D). For Group 1 mice that received vectors carrying GFP-Luciferase, expression driven by CMV promoter, the luciferase expression was observed in various tissues and predominantly in lungs, liver and muscle (See FIG. 17A). In contrast, luciferase expression was restricted to liver in Group 3 mice, which received luciferase vector in which the expression was controlled by TBG promoter (see FIG. 17B). In Group 2 mice, the level of luciferase expression was elevated by more than 2 logs compared to level of pre-induction, and the expression is predominantly in liver and muscle (see FIG. 17C). In Group 4 mice, more than 100 fold of luciferase expression was induced and restricted in the liver, compared to pre-inducement (see FIG. 17D).

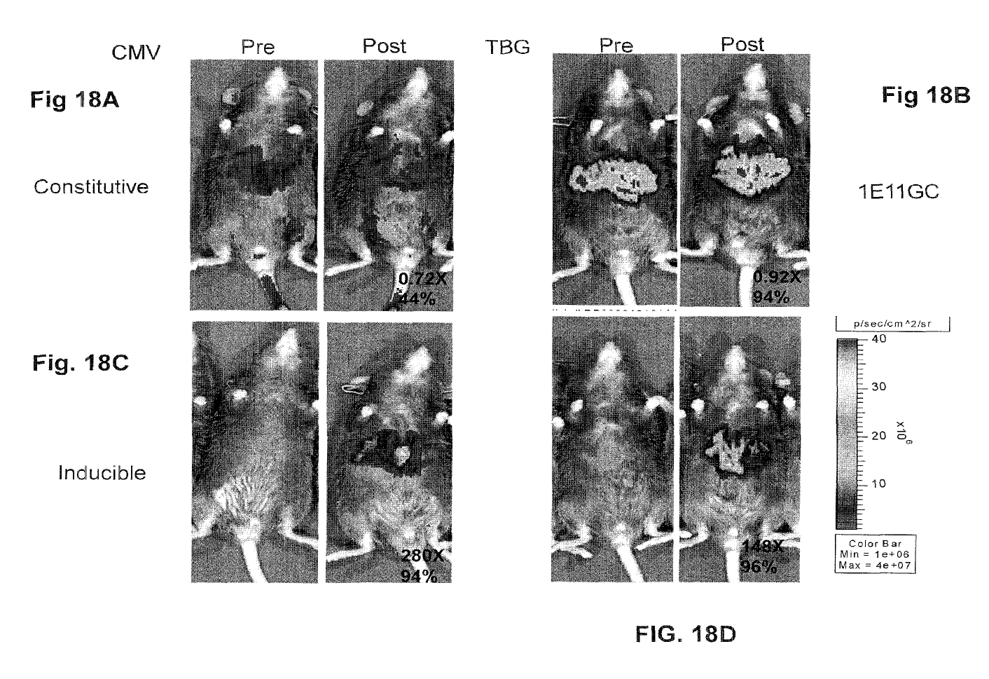

The mice that received 1×10¹¹ particles of viruses, show results similar to that of high dose groups but with lower level of expression upon induction, and predominantly in liver (see FIGS. 18A-D).

### Conclusions:

1. The dimerizer-inducible system is robust with peak level of luciferase expression more than 2 logs over baseline and back to close to baseline within a week (not shown).

2. Liver is the most efficient tissue to be infected when viruses were given IV.

3. Liver is also the most efficient tissue to be cotransduced with 2 viruses which is critical for the dimerizer-inducible system to work.

4. The luciferase expression regulated by that dimerizer-inducible system with transcription factor expression controlled by CMV promoter is significantly higher in mouse liver than expression coming from CMV promoter without regulation. This indicated that inducible promoter is a stronger promoter in liver once it is activated compared to the CMV promoter.

5. Luciferase expression was detected specifically in liver upon induction by rapamycin in mice receiving vectors carrying the inducible TBG promoter system. Luciferase expression mediated by the liver-specific regulatable vectors was completely dependent upon induction by rapamycin and the peak level of luciferase expression is comparable to that under the control of TBG promoter. This study confirmed that liver specific gene regulation can be achieved by AAV mediated gene delivery of liver specific dimerizer-inducible system.

### 11. EXAMPLE 6

### PITA for Age-Related Macular Degeneration (AMD) Therapy

Intravitreal administration of a monoclonal antibody has proven to be an effective therapy for AMD to slow down disease progression and improve visual acuity in a subpopulation of patients. A key limitation of this approach, however, is the requirement for repeated intravitreal injections. Gene therapy has the potential to provide long term correction and a single injection should be sufficient to achieve a therapeutic effect. FIGS. 19 A-C show PITA DNA constructs for treating AMD, containing transgene units comprising a VEGF antagonist, such as an anti-VEGF antibody (Avastin heavy chain (AvastinH) and Avastin light chain (AvastinL); FIGS. 19B and 19C) or a soluble VEGF receptor (sFlt-1; FIG. 19A). Vectors comprising these DNA constructs can be delivered via subretinal injection at the dose of 0.1-10 mg/kg. Ablation of transgene expression can be achieved by oral dimerizer administration if adverse effects of long term anti-VEGF therapy are observed.

### 12. EXAMPLE 7

### PITA for Liver Metabolic Disease Therapy

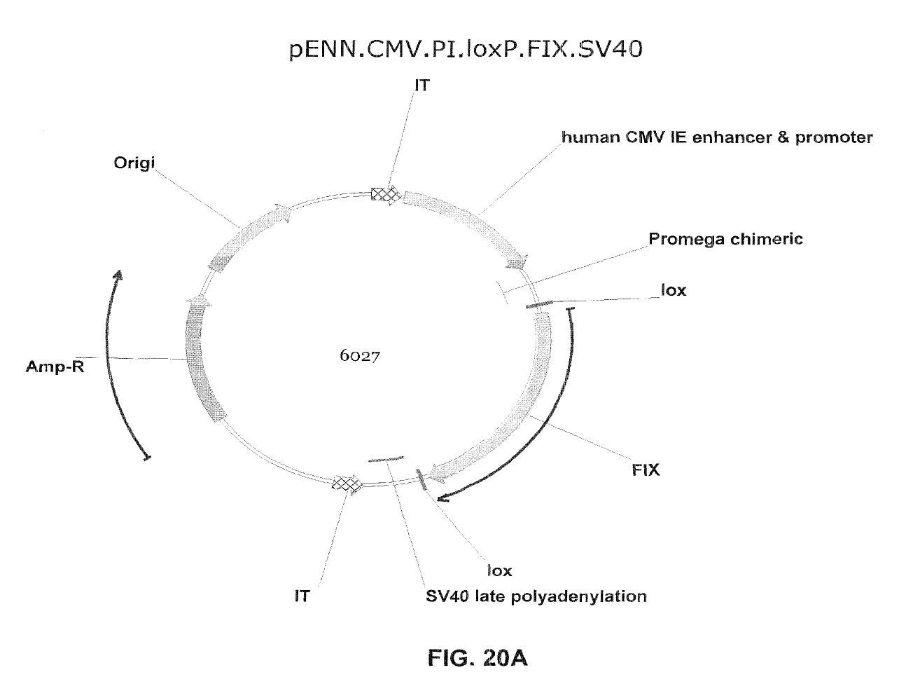

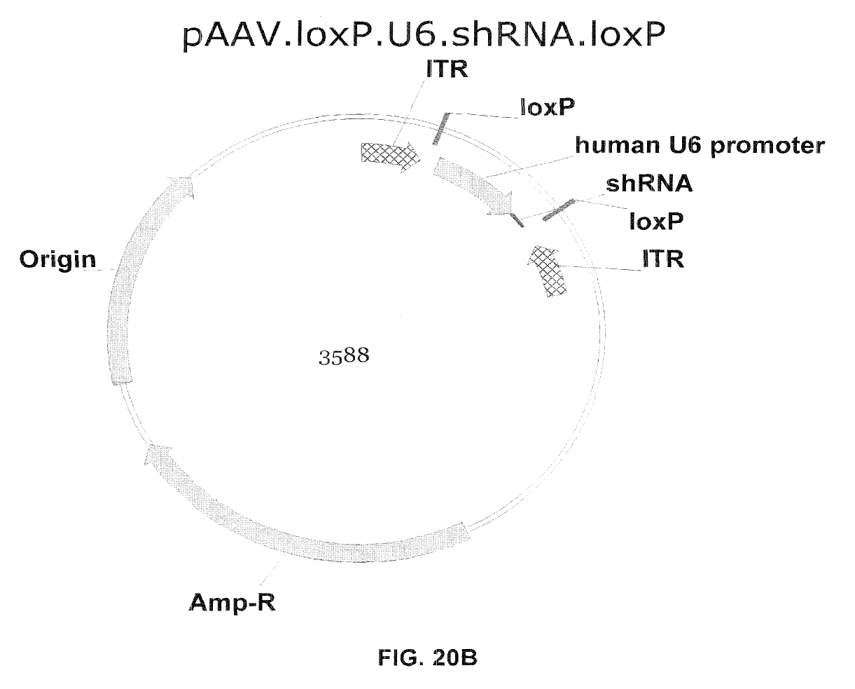

PITA is potentially useful for treating liver metabolic disease such as hepatitis C and hemophilia. FIG. 20A shows a PITA construct for treating hemophilia A and/or B, containing a transgene unit comprising Factor IX. Factor VIII can also be delivered for treatment of hemophilia A and B respectively (Factor VIII and IX for hemophilia A and B, respectively). The therapy could be ablated in patients if inhibitor formation occurs. FIG. 20B shows a PITA construct for delivery of shRNA targeting the IRES of HCV. A vector comprising this construct could be injected via a mesenteric tributary of portal vein at the dose of 3×10¹² GC/kg. The expression of shRNA can be ablated if nonspecific toxicity of RNA interference arises or the therapy is no longer needed.

### 13. EXAMPLE 8

### PITA for Heart Disease Therapy

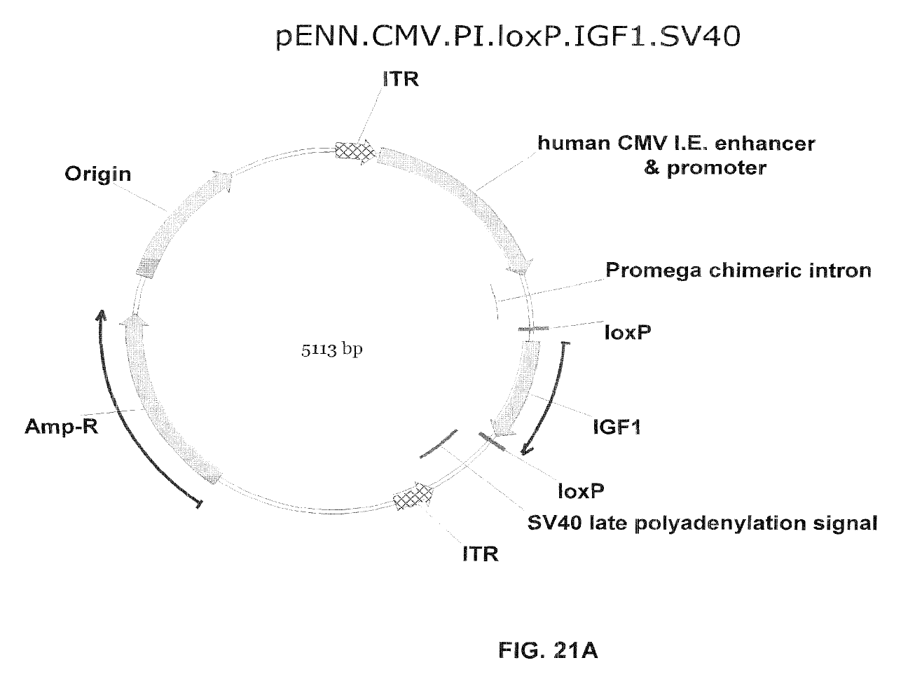

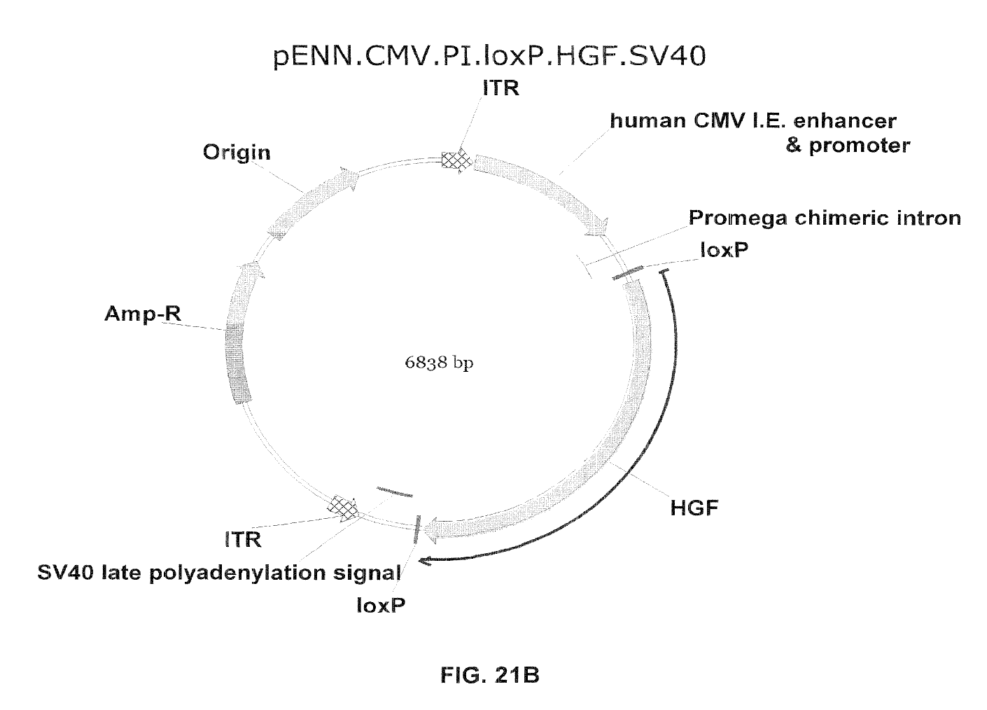

PITA could be utilized for heart disease applications including, but not limited to, congestive heart failure (CHF) and myocardial infarction (MI). The treatment of CHF could involve the delivery of insulin like growth factor (IGF) or hepatocyte growth factor (HGF) using the constructs shown in FIGS. 21A and 21B. For the treatment of myocardial infarction, delivery of genes in the early stages of MI could protect the heart from the deleterious effects of ischemia but allow ablation of the therapy when no longer required. Therapeutic genes for this approach include heme oxygenase-1 (HO-1) which can function to limit the extent of ischemic injury. Delivery methods for vector-mediated gene delivery to the heart include transcutaneous, intravascular, intramuscular and cardiopulmonary bypass techniques. For the human, the optimal vector-mediated gene delivery protocol would likely utilize retrograde or ante grade trans coronary delivery into the coronary artery or anterior cardiac vein.

### 14. EXAMPLE 9

### PITA for Central Nervous System (CNS) Disease Therapy

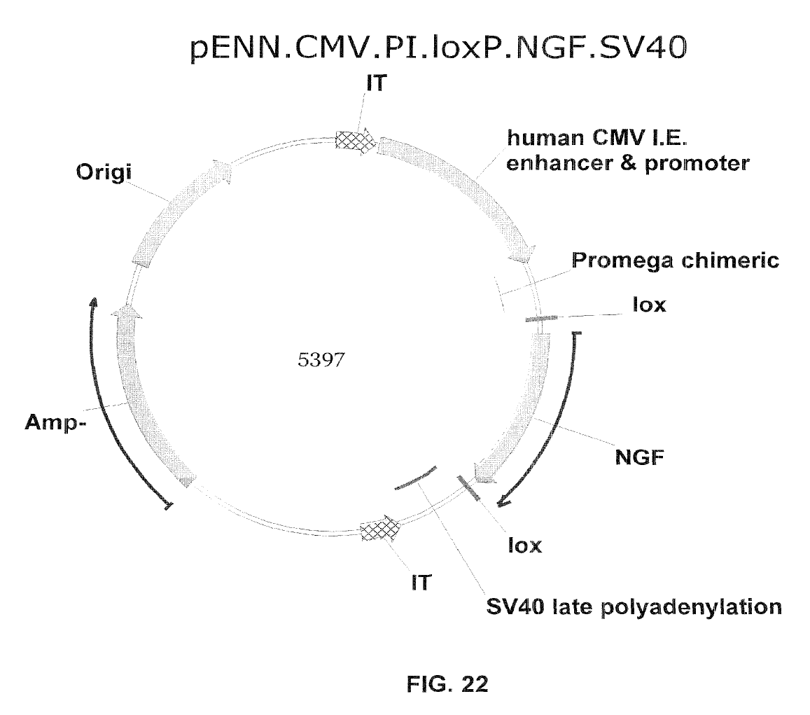

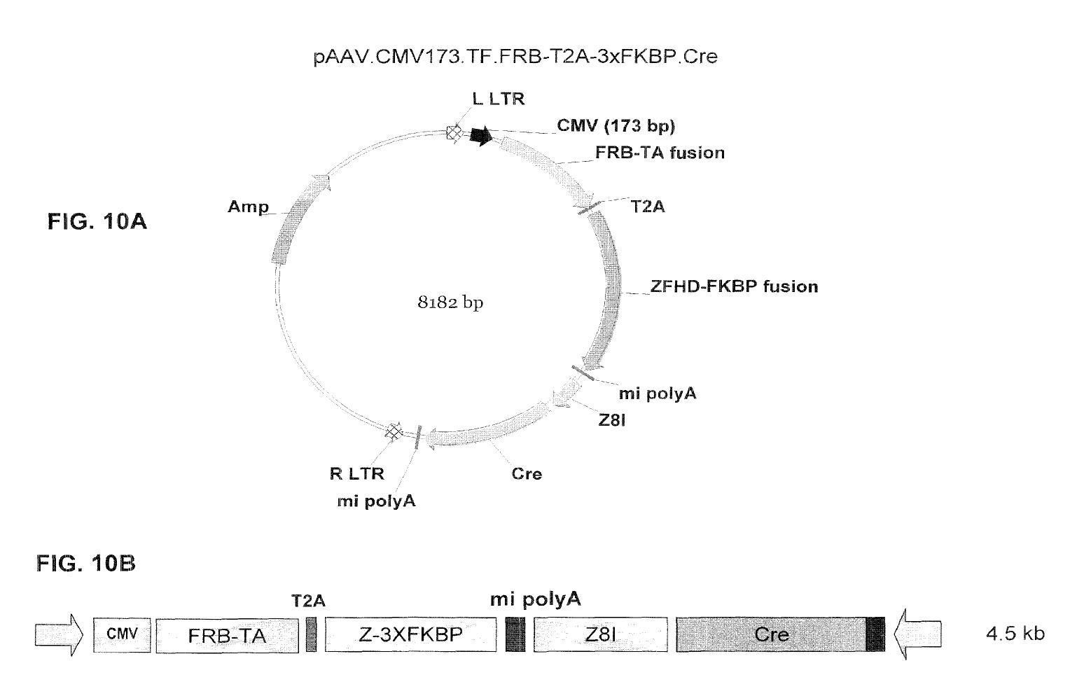

Attractive candidates for the application of PITA in the central nervous system include neurotrophic factors for the treatment of Alzheimer's disease, Parkinson's disease, amyotrophic lateral sclerosis (ALS), Huntington's disease and ocular diseases. FIG. 22 shows a PITA construct for treating Alzheimer's disease, containing a transgene unit comprising nerve growth factor (NGF). AAV vector-mediated gene delivery of NGF, is currently being studied in a Phase I clinical trial conducted by Ceregene for the treatment of Alzheimer's disease. NGF is a neurotrophic factor, which has been shown to be effective in reducing cholinergic cell loss in animal models of neurodegenerative disease and may be effective in preventing loss of memory and cognitive abilities in patients with AD. The delivery method for the approach consists of bilateral, stereotactic injection to target the basal forebrain region of the brain containing the nucleus basalis of Meynert (NBM). Due to the potential for side-effects resulting in the need to end treatment, further engineering the construct to include PITA is warranted.

The application of PITA in the central nervous system for the treatment of epilepsies could also be of value both due to the potential to ablate gene expression once the issue surrounding the seizures becomes resolved as well as due to the limited alternative approaches available for the treatment of epilepsies that are unresponsive to drug therapy and surgically difficult to treat. In these cases, in particular, delivery methods involving sterotactic injection of vectors expressing therapeutic genes, would be far less invasive than alternative surgical treatments. Candidates for gene expression could include galanin, neuropeptide Y (NPY) and glial cell line-derived neurotrophic factor, GDNF, which have been shown to have therapeutic effects in animal models of epilepsy. Other applications include to deliver nerve growth factor (NGF) for Alzheimer's and aromatic L-amino acid decarboxylase (ADCC) for Parkinson's Disease.

### 15. EXAMPLE 10

### PITA for HIV Therapy

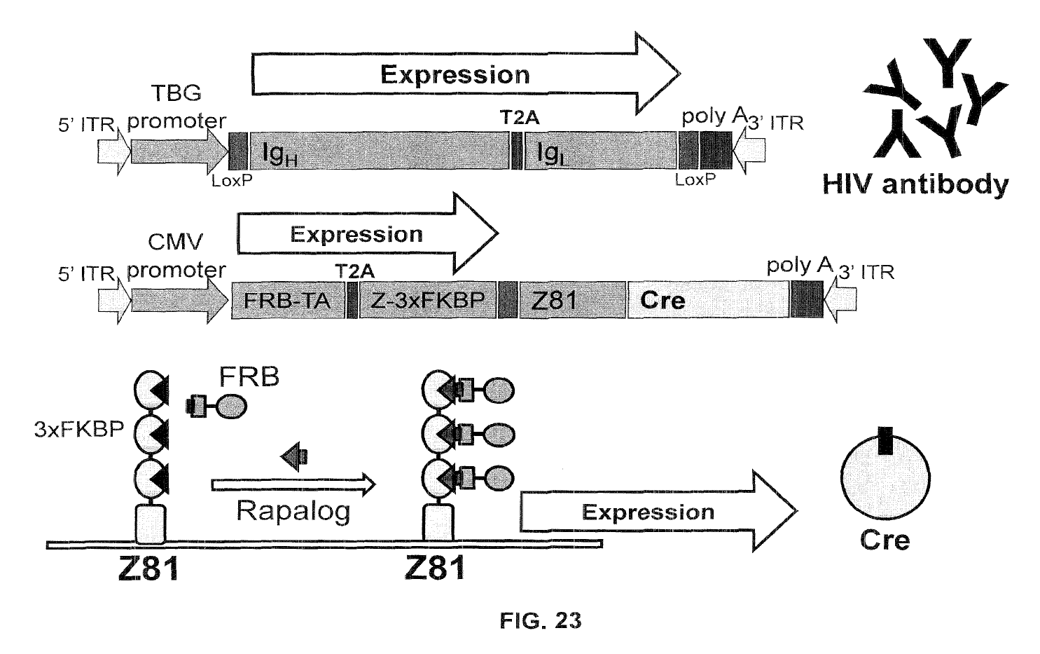

Naturally induced neutralizing antibody against HIV has been identified in the sera of long term infected patients. As an alternative to active vaccine approaches, which have resulted in inefficient induction but sufficient levels of neutralizing antibody delivered by AAV, PITA is a promising approach to deliver anti-HIV neutralizing antibody for passive immunity therapy. See FIG. 23. The construct design is similar to avastin gene delivery for AMD therapy (see FIGS. 19B and 19C). A vector comprising a construct encoding an antibody regulated by the liver specific promoter (TBG) could be injected into the liver at a dose of 3×10¹² GC/kg. Alternatively, a vector comprising a construct carrying a ubiquitous C137 promoter driving antibody expression could be delivered by intramuscular injection at a dose of 5×10¹² GC/mL for up to 20 injections into the quadriceps or biceps muscle. The therapy can be ablated if it is no longer needed or if toxicity develops due to induction of anti-drug antibody.

### 16. EXAMPLE 11

The DNA constructs described in the following example may be used to prepare replication-defective AAV viruses and virus compositions according to the invention.

Open reading frames encoding for various endonucleases were codon optimized and de novo synthesized by GeneArt. Ablator expression and target plasmids were produced using standard molecular biological cloning techniques. Transfections were performed in HEK293 cells using Lipofectamine™ 2000 transfection reagent (Life Technologies). All transfections were performed using optimal transfection conditions as defined in transfection reagent protocol. Briefly, 200-250 ng plasmid DNA (excluding transfection control plasmid) was complexed with lipofectamine and added to cells in 96 well plates. DNA quantities were consistent across all conditions by supplementation with an unrelated plasmid containing the same promoter as test plasmids. Transfection complexes were incubated with cells for 4-6 hours as transfection reagent protocol before the addition of FBS supplemented media. Transfected cells were incubated at 37° C. for 24-72 hours. Following incubation, cells were assayed for reporter gene expression using Promega Dual Luciferase detection kit according to the manufacturer's instructions on a BioTek Clarity platereader and renilla luciferase was used to control for transfection efficiency. All samples were performed in quadruplicate and standard errors of the mean were calculated.

A. Coexpression of Wild-Type FokI Ablates Expression of Transgene More Effectively than Delivery of FokI Protein

The amino acid sequence of the FokI enzyme is provided in SEQ ID NO: 12, wherein amino acids 1 to 387 are the DNA binding domain and amino acids 387 to 584 are the catalytic domain. The codon optimized FokI sequence is provided in SEQ ID NO:1.

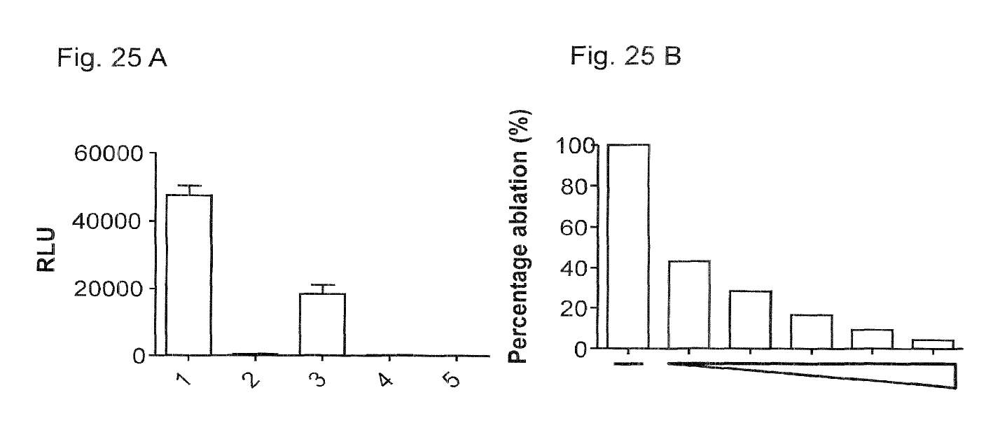

FIG. 25 illustrates that wild-type FokI effective ablated expression of the luciferase reporter gene following contrasfection into HEK295 cells (FIG. 25A bar 2), while only partial ablation was observed when FokI protein was delivered to the cells (FIG. 25A, bar 3).

In a dose-dependent experiment, the FokI expression vector contained the Fold catalytic domain fused to a zinc finger DNA binding domain (ZFHD). This construct, which is 963 bp, is provided in SEQ ID NO: 21 and is composed of base pairs 1 to 366 bp ZFHD, 367 to 372 bp linker, and 373 to 963 bp FokI catalytic domain. The resulting expression product comprises amino acids 1 to 122 (ZFHD), amino acids 123-124 are a linker and amino acids 125 to 321 are from the FokI catalytic domain. FIG. 25B illustrates that increasing the concentration of FokI resulted in dose dependent ablation of Luc reporter. No ablation sites were required to be engineered into the transcription unit containing the transgene in this illustration, as luciferase contains multiple native FokI sites.

This provides support for the use of the PITA system using a transfected FokI enzyme directed to specific ablation sites in a transcription unit containing a transgene for delivery to the cell.

B. Chimeric Engineered FokI Tethered to Non-Cognate Recognition Site on the DNA by the Zinc Finger—Homeodomain Effectively Ablates Expression of Luc Reporter Gene

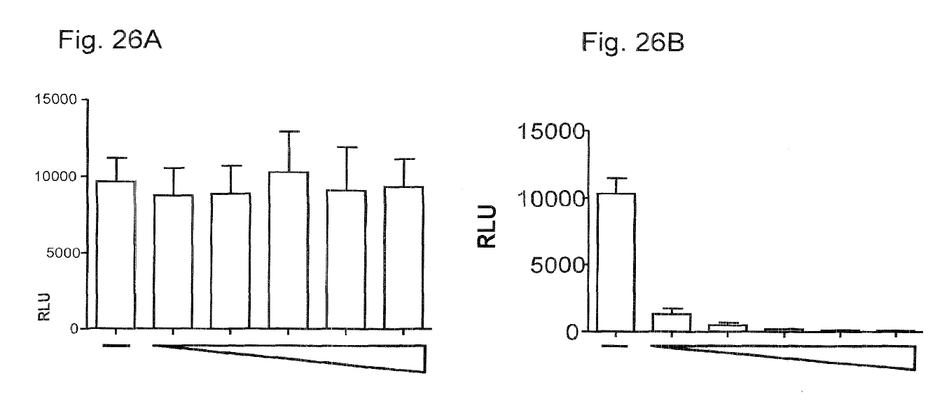

The plasmid constructs in this example contains either the Fold catalytic domain (198 amino acids (SEQ ID NO: 14), corresponding to amino acids 387 to 584 of the full-length protein) (untethered FokI) or a ZFHD-FokI catalytic domain of 963 bp as described in Part A above (tethered FokI). Even at the highest concentration, the catalytic domain of FokI which is un-tethered to DNA does have no effect on expression of Luc reporter gene (FIG. 26A). Chimeric engineered FokI tethered to DNA via fusion with ZFHD effectively ablated expression of luciferase reporter in a dose dependent manner when increasing concentrations of ZF-HD-FokI expression plasmid were cotransfected into HEK293 cells (FIG. 26B).

This supports the use of the PITA system and the additional safety element provided by a chimeric enzyme directed to specific ablation sites in a transcription unit containing a transgene for delivery to the cell.

C. DNA Binding Specificity of Chimeric FokI can be Reproducibly Changed by Fusion with Various Classes of Heterologous DNA Binding Domains and Ablation of Target Transgene can be Further Improved by Addition of Heterologous NLS

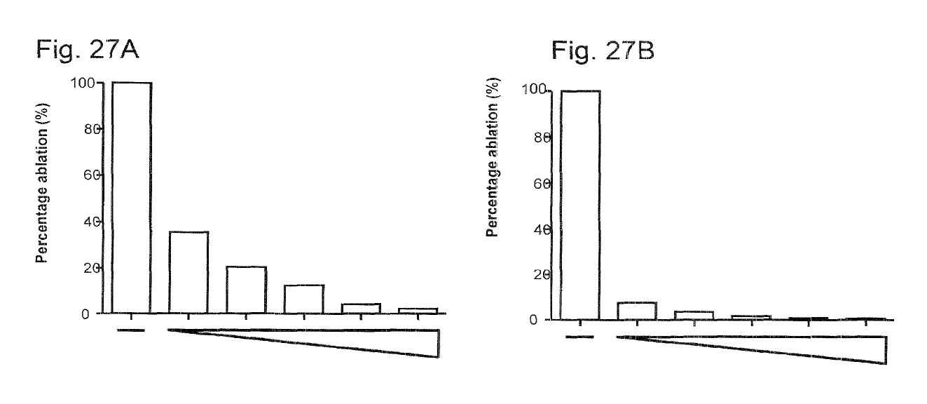

This example illustrates that the zinc finger homeodomain (ZFHD) is not the only domain suitable for altering the specificity of ablation mediated by a chimeric engineered enzyme. FokI effectively ablated expression of luciferase reporter in a dose dependent manner when HTH DNA binding domain was fused to FokI catalytic domain (FIG. 27A). In a separate experiment (FIG. 27B), the activity of HTH-FokI was further improved by adding heterologous NLS at the N-terminus of the HTH-Fold coding sequence.

The HTH-FokI Catalytic domain (SEQ ID NO:5), is composed of 1-171 bp HTH from Gin (a serine recombinase), a linker (bp 172-177), and a FokI catalytic domain (178-768 bp) derived from codon-optimized FokI. The resulting chimeric enzyme (SEQ ID NO: 6) contains aa 1-57 of HTH from Gin, a linker (aa 58-59), and a FokI catalytic domain (amino acids 60-256).

FIGS. 27A-27B are bar charts illustrating that the DNA binding specificity of chimeric FokI can be reproducible changed by fusion with another classes of heterologous DNA binding domains and ablation of target transgene can be further improved by the additional of a heterologous nuclear localization signal (NLS). FIG. 27A illustrates the results of co-transfection of pCMV.Luciferase with increasing concentrations of an expression plasmid encoding FokI tethered to DNA via an HTH fusion (6.25, 12.5, 25, 50, and 100 ng). The first bar is a control showing 50 ng pCMV.Luciferase alone. FIG. 27B pCMV.Luciferase with increasing concentrations of an expression plasmid encoding an HTH-FokI fusion, which further has a NLS at its N-terminus.

### 17. EXAMPLE 12

Although not illustrated here, other chimeric enzymes have been made using the techniques described herein:
An AAV plasmid containing SV40 T-Ag NLS-Helix-turn-helix (HTH) from Gin (192 bp, SEQ ID NO:7), which includes the nuclear localization signal (1-24 bp) of SV40 T-Ag and HTH from Gin, a serine recombinase (25-192 bp). In the resulting enzyme (SEQ ID NO:8), amino acids 1-8 are from the SV40 T-Ag NLS and amino acids 9-64 are the HTH from Gin;
An AAV plasmid containing SV40 T-Ag NLS-HTH-FokI Catalytic domain (789 bp, SEQ ID NO:9), which includes the SV40 T-Ag NLS (bp 1-24), the HTH from Gin (bp 25-192), a linker (bp 193-198), and the catalytic domain of the FokI (bp 199-789), In the resulting chimeric enzyme (SEQ ID NO:10), amino acids 1-8 are from the SV40 T-Ag NLS, amino acids 9-64 are HTH from Gin, amino acids 65-66 are linker residues, and amino acids 67-263 are the FokI catalytic domain.
An AAV plasmid containing a SV40 T-Ag NLS-ZFHD-FokI catalytic domain (984 bp) was prepared (SEQ ID NO: 23), which includes the SV40 T-Ag NLS (bp 1-24), the zinc finger homodomain (bp 25-387), a linker (bp 388-393), and the Fold catalytic domain (bp acids 394-984). In the resulting chimeric enzyme (SEQ ID NO: 21, 328 aa), amino acids 1-8 are the SV40 T-Ag NLS, amino acids 9-129 are the ZFHD, amino acids 130-131 are linker residues, and amino acids 132-138 are Fold catalytic domain.

These and other constructs can be used to prepare viruses according the method of the invention for use in a virus composition and the PITA system.

### 18. EXAMPLE 13

### Use of Replication-Defective AAV Virus Composition in Treatment of HIV

This composition could be potentially used as a safety mechanism in the treatment of HIV. Recently, broadly neutralizing antibodies from long-term non-progressors, individuals which maintain an HIV⁺ status for several decades without progression to AIDS, have been identified by several research groups.

All coding regions of the neutralizing antibody to HIV (HIV NAb) are placed between the inverted terminal repeats (ITRs) of the AAV. If the overall size of the constructs are below 4.7 kb (including the two ITRs), they are packaged into the AAV capsid. The AAV serotype capsid chosen will depend of the level of gene expression, the method of delivery and the extent of biodistribution from the injection site required. In addition, the constitutive promoters used for expression of the HIV NAb (and potentially the parts of the inducible system in the one small molecule situation) would depend on the tissue type targeted. In the following example of a potential clinical study the vector serotype chosen would be AAV8 administered by intravenous injection which would enable utilization of the liver specific promoter TBG.

In HIV⁺ patients, administration of AAV vectors expressing one or more of these HIV neutralizing antibodies would lead to long-term, high level expression of one or more broadly HIV NAb and would reduce viral load and potentially prevent acquisition of HIV. In this situation, individuals would receive intravenous injection of two AAV vectors at a dose of 5×10¹² genome copies/kilogram of each vector. Contained within the two AAV vectors would be the HIV neutralizing antibody under control of a constitutive promoter, allowing expression to occur rapidly following administration of the vector.

A. Heterodimer and Two Small Molecules

Following the first signs of potential toxicity to the HIV NAb, the first small molecule drug would be administered to induce expression of the components of the inducible system, in this case the DNA binding domain linked to FKBP and FRAP_{L} linked to the catalytic domain of a endonuclease enzyme. This would allow the system to be primed for action should further toxicity to the HIV NAb develop. If toxicity levels continue to rise then initiation of endonuclease activity would be induced by administration of a second small molecule drug which would lead to the formation of an active enzyme and ablation of HIV NAb gene expression.

B. Heterodimer and One Small Molecule

Also under the control of constitutive expression would be the elements of the rapamycin inducible system, FKBP and FRAP_{L}. Following administration of the AAV vectors, patients would be closely monitored at regular intervals for several years. If toxicity to the HIV NAb develops then delivery of rapamycin or a rapalog would be implemented. IV administration of 1 mg/kg rapamycin/rapalog in the first instance with the potential to increase to repeated dosing would be administered to ablate expression of the HIV antibody.

Toxicity and HIV antibody levels would be closely monitored until expression of the HIV NAb had reached undetectable levels. Therefore, the ablation of gene expression of the HIV NAb would provide a safety switch to ablate gene expression should insurmountable toxicity occur.

All publications, patents, and patent applications cited in this application, as well as priority application U.S. Patent Application No. 61/318,755 and the Sequence Listing, are hereby incorporated by reference in their entireties as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

1. A replication-defective adeno-associated virus composition suitable for use in human subjects comprising an AAV with a capsid having packaged therein:
(a) a first transcription unit that encodes a therapeutic product in operative association with a promoter that controls transcription, said first transcription unit containing an ablation recognition site; and
(b) a second transcription unit that encodes an ablator specific for the ablation recognition site in operative association with a promoter, wherein transcription and/or ablation activity is controlled by a pharmacological agent.

2. The replication-defective virus composition according to claim 1, wherein said first transcription unit contains more than one ablation recognition site.

3. The replication-defective virus composition according to claim 1, wherein the composition comprises more than one ablation recognition site, said more than one ablation recognition site comprising a first ablation recognition site and a second ablation recognition site which differs from said first ablation recognition site, said virus further comprising a first ablator specific for the first ablation recognition site and a second ablator specific for the second recognition site.

4. The replication-defective virus composition of claim 1 in which transcription of the ablator is controlled by a regulatable system.

5. The replication-defective virus composition of claim 4 in which the regulatable system is selected from a tet-on/off system, a tetR-KRAB system, a mifepristone (RU486) regulatable system, a tamoxifen-dependent regulatable system, a rapamycin-regulatable system, or an ecdysone-based regulatable system.

6. (canceled)

7. The replication-defective virus composition of claim 1 in which the ablator is Cre and the ablation recognition site is loxP, or the ablator is FLP and the ablation recognition site is FRT.

8. The replication-defective virus composition according to claim 1, wherein the ablator is a chimeric engineered endonuclease, wherein the virus composition comprises (i) a first sequence comprising the DNA binding domain of the endonuclease fused to a binding domain for a first pharmacological agent; and wherein the virus composition further comprises (ii) a second sequence encoding the nuclease cleavage domain of the endonuclease fused to a binding domain for the first pharmacological agent, wherein the first sequences (i) and the second sequence (ii) are each in operative association with at least one promoter which controls expression thereof.

9. The replication-defective virus composition according to claim 8, wherein the chimeric engineered endonuclease is contained within a single bicistronic open reading frame in the second transcription unit, said transcription unit further comprising a linker between (i) and (ii).

10. The replication-defective virus according to claim 8, wherein the sequence (i) and/or the sequence (ii) has an inducible promoter.

11. The replication-defective virus composition according to claim 8, wherein the chimeric engineered endonuclease is contained within separate open reading frames.

12. The replication-defective virus composition according to claim 8, wherein each of the first sequence and the second sequence are under the control of a constitutive promoter and the ablator is bioactivated by the first pharmacological agent.

13-15. (canceled)

16. The replication-defective virus composition according to claim 12, wherein said ablator is a chimeric FokI enzyme.

17. (canceled)

18. The virus composition of claim 49, wherein the first promoter of (c) and the second promoter of (d) are independently selected from a constitutive promoter and an inducible promoter.

19. The replication-defective virus composition of claim 18, wherein the first and second promoters are both constitutive promoters and the pharmacological agent is a dimerizer that dimerizes the domains of the transcription factor.

20. The replication-defective virus composition of claim 18, wherein one of the first promoter and the second promoters is an inducible promoter.

21-26. (canceled)

28. A recombinant DNA construct comprising a first and second transcription unit flanked by packaging signals of an AAV viral genome, in which:
(a) a first transcription unit that encodes a therapeutic product in operative association with a promoter that controls transcription, said first transcription unit containing at least one ablation recognition site; and
(b) a second transcription unit that encodes an ablator specific for the at least one ablation recognition site in operative association with a promoter that induces transcription in response to a pharmacological agent.

29. The DNA construct of claim 28 in which the packaging signals flanking the transcription units are an AAV 5′ inverted terminal repeats (ITR) and a AAV 3′ ITR.

30. The DNA construct of claim 29 in which the AAV ITRs are AAV1, AAV6, AAV7, AAV8, AAV9 or rh10 ITRs.

31. The DNA construct of claim 29 in which the first transcription unit is flanked by AAV ITRs, and the second, third and fourth transcription units are flanked by AAV ITRs.

32. The DNA construct of claim 29 in which the transcription units are contained in two or more DNA constructs.

33. (canceled)

34. The DNA construct of claim 28 in which the promoter that controls transcription of the therapeutic product is a constitutive promoter, a tissue-specific promoter, a cell-specific promoter, an inducible promoter, or a promoter responsive to physiologic cues.

35-44. (canceled)

45. A genetically engineered cell comprising a DNA construct according to claim 28, which cell is selected from a plant, bacterial or non-human mammalian cell.

46-48. (canceled)

49. An adeno-associated virus (AAV) composition suitable for use in human subjects in which the AAV comprises:
(a) a first transcription unit that encodes a therapeutic product in operative association with a promoter that controls transcription, said first transcription unit containing an ablation recognition site;
(b) a second transcription unit that encodes an ablator specific for the ablation recognition site in operative association with a promoter, wherein transcription and/or ablation activity is controlled by a pharmacological agent;
(c) a third transcription unit encoding a dimerizable domain of a transcription factor that regulates an inducible promoter for the ablator, in which the third transcription unit encodes the DNA binding domain of the transcription factor fused to a binding domain for the pharmacological agent in operative association with a first promoter; and
(d) a fourth transcription unit encoding a dimerizable domain of said transcription factor of (c) that said inducible promoter for the ablator, in which the fourth transcription unit encodes the activation domain of the transcription factor fused to a binding domain for the pharmacological agent in operative association with a second promoter.

50. The AAV composition according to claim 49, wherein the coding sequence for the ablator further comprises a nuclear localization signal located 5′ or 3′ to the ablator coding sequence.

51. The AAV composition according to claim 49, in which the third and fourth transcription units are a bicistronic unit containing an IRES or furin-2A.

52. The AAV composition according to claim 49, in which the pharmacological agent is rapamycin or a rapalog.

53. The AAV composition according to claim 49, in which the first transcription unit is on a first AAV and the second transcription unit is on a second AAV in the composition.

54. The AAV composition according to claim 53, in which the second AAV comprises a second ablator.

55. The AAV composition according to claim 49, wherein the DNA binding domain is selected from the group consisting of a zinc finger, helix-turn-helix, a HMG-Box, Stat proteins, B3, helix-loop-helix, winged helix-turn-helix, leucine zipper, a winged helix, POU domains, and a homeodomain.

56. The AAV composition according to claim 49, in which the ablator is selected from the group consisting of an endonuclease, a recombinase, a meganuclease, or a zinc finger endonuclease that binds to the ablation recognition site in the first transcription unit and excises or ablates DNA and an interfering RNA, a ribozyme, or an antisense that ablates the RNA transcript of the first transcription unit, or suppresses translation of the RNA transcript of the first transcription unit.


SEQUENCE LISTING (58 sequences)

SEQ ID NO: 1 - DNA, 579 bp, Artificial Sequence
  modified Gin enzyme
atgctgatcggctacgtgcgggtgtccaccaacgaccagaacaccgacctgcagcggaacgccctggtctgcgccggctgcgagcagatcttcgaggacaagctgagcggcacccggaccgacagacccggactgaagcgggccctgaagcggctgcagaaaggcgacaccctggtggtctggaagctggaccggctgggcagatccatgaagcacctgatcagcctggtcggagagctgagagagcggggcatcaacttcagaagcctgaccgacagcatcgacaccagcagccctatgggccggttcttcttctacgtgatgggcgccctggccgagatggaaagagagctgatcatcgagcggacaatggccggactggccgctgcccggaacaagggcagaatcggcggcagaccccctaggctgaccaaggccgagtgggaacaggctggcagactgctggcccagggaatcccccggaaacaggtggccctgatctacgacgtggccctgagcaccctgtataagaagcaccccgccaagagagcccacatcgagaacgacgaccggatcaac

SEQ ID NO: 2 - protein, 193 aa, Artificial Sequence
  Synthetic Construct
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELIIERTMAGLAAARNKGRIGGRPPRLTKAEWEQAGRLLAQGIPRKQVALIYDVALSTLYKKHPAKRAHIENDDRIN

SEQ ID NO: 3 - DNA, 171 bp, Artificial Sequence
  modified Gin enzyme
atgggcagaccccctaggctgaccaaggccgagtgggaacaggctggcagactgctggcccagggaatcccccggaaacaggtggccctgatctacgacgtggccctgagcaccctgtataagaagcaccccgccaagagagcccacatcgagaacgacgaccggatcaac

SEQ ID NO: 4 - protein, 57 aa, Artificial Sequence
  Synthetic Construct
MGRPPRLTKAEWEQAGRLLAQGIPRKQVALIYDVALSTLYKKHPAKRAHIENDDRIN

SEQ ID NO: 5 - DNA, 768 bp, Artificial Sequence
  modified enzyme from Gin and FokI
atgggcagaccccctaggctgaccaaggccgagtgggaacaggctggcagactgctggcccagggaatcccccggaaacaggtggccctgatctacgacgtggccctgagcaccctgtataagaagcaccccgccaagagagcccacatcgagaacgacgaccggatcaacggtaccaagcagctggtgaaaagcgagctggaagagaagaagtccgagctgcggcacaagctgaaatacgtgccccacgagtacatcgagctgatcgagatcgcccggaaccccacccaggacagaatcctggaaatgaaggtcatggaatttttcatgaaggtgtacggctaccggggcgagcacctgggcggcagcagaaaacccgacggcgccatctacaccgtgggcagccccatcgactacggcgtgatcgtggacaccaaggcctacagcggcggctacaacctgcccatcggacaggccgacgagatgcagagatacgtggaagagaaccagacccggaacaagcacatcaaccccaacgagtggtggaaggtgtaccccagcagcgtgaccgagttcaagttcctgttcgtgtccggccacttcaagggcaactacaaggcccagctgacccggctgaaccacatcaccaactgcaacggcgctgtgctgagcgtggaagaactgctgatcggcggcgagatgatcaaggccggcaccctgaccctggaagaagtgcggcggaagttcaacaacggcgagatcaacttc

SEQ ID NO: 6 - protein, 256 aa, Artificial Sequence
  Synthetic Construct
MGRPPRLTKAEWEQAGRLLAQGIPRKQVALIYDVALSTLYKKHPAKRAHIENDDRINGTKQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 7 - DNA, 192 bp, Artificial Sequence
  modified enzyme from SV40 and Gin
atgcccaagaagaagagaaaggtgggcagaccccctaggctgaccaaggccgagtgggaacaggctggcagactgctggcccagggaatcccccggaaacaggtggccctgatctacgacgtggccctgagcaccctgtataagaagcaccccgccaagagagcccacatcgagaacgacgaccggatcaac

SEQ ID NO: 8 - protein, 64 aa, Artificial Sequence
  Synthetic Construct
MPKKKRKVGRPPRLTKAEWEQAGRLLAQGIPRKQVALIYDVALSTLYKKHPAKRAHIENDDRIN

SEQ ID NO: 9 - DNA, 789 bp, Artificial Sequence
  modified enzyme from SV40, Gin and FokI
atgcccaagaagaagagaaaggtgggcagaccccctaggctgaccaaggccgagtgggaacaggctggcagactgctggcccagggaatcccccggaaacaggtggccctgatctacgacgtggccctgagcaccctgtataagaagcaccccgccaagagagcccacatcgagaacgacgaccggatcaacggtaccaagcagctggtgaaaagcgagctggaagagaagaagtccgagctgcggcacaagctgaaatacgtgccccacgagtacatcgagctgatcgagatcgcccggaaccccacccaggacagaatcctggaaatgaaggtcatggaatttttcatgaaggtgtacggctaccggggcgagcacctgggcggcagcagaaaacccgacggcgccatctacaccgtgggcagccccatcgactacggcgtgatcgtggacaccaaggcctacagcggcggctacaacctgcccatcggacaggccgacgagatgcagagatacgtggaagagaaccagacccggaacaagcacatcaaccccaacgagtggtggaaggtgtaccccagcagcgtgaccgagttcaagttcctgttcgtgtccggccacttcaagggcaactacaaggcccagctgacccggctgaaccacatcaccaactgcaacggcgctgtgctgagcgtggaagaactgctgatcggcggcgagatgatcaaggccggcaccctgaccctggaagaagtgcggcggaagttcaacaacggcgagatcaacttc

SEQ ID NO: 10 - protein, 263 aa, Artificial Sequence
  Synthetic Construct
MPKKKRKVGRPPRLTKAEWEQAGRLLAQGIPRKQVALIYDVALSTLYKKHPAKRAHIENDDRINGTKQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 11 - DNA, 1752 bp, Artificial Sequence
  modified FokI enzyme
atgtttctgagcatggtgtccaagatccggaccttcggctgggtgcagaaccccggcaagttcgagaacctgaagcgggtggtgcaggtgttcgaccggaacagcaaggtgcacaacgaagtgaagaacatcaagatccccacactggtgaaagagagcaagatccagaaagaactcgtcgccatcatgaaccagcacgacctgatctacacctacaaagaactggtcggaaccggcaccagcatcagaagcgaggccccctgcgacgccatcattcaggccacaatcgccgaccagggcaacaagaagggctacatcgacaactggtccagcgacggcttcctgagatgggcccacgccctgggcttcatcgagtacatcaacaagagcgacagcttcgtgatcaccgacgtgggcctggcctacagcaagagcgccgatggcagcgccattgagaaagagatcctgatcgaggccatcagcagctacccccctgccatcagaatcctgaccctgctggaagatggccagcacctgaccaagttcgacctgggcaagaacctgggcttctccggcgagagcggcttcaccagcctgcccgagggaatcctgctggacaccctggccaacgccatgcccaaggacaagggcgagatccggaacaactgggagggcagcagcgataagtacgccagaatgatcggcggctggctggacaagctgggcctggtcaaacaggggaagaaagagttcatcattcccaccctgggcaagcccgacaacaaagagtttatcagccacgccttcaagatcacaggcgagggcctgaaggtgctgcggagagccaagggcagcaccaagttcacacgggtgcccaagcgggtgtactgggagatgctggccaccaacctgaccgacaaagaatacgtgcggaccagacgggccctgatcctggaaatcctgattaaggccggcagcctgaagatcgagcagatccaggacaacctgaagaagctgggctttgacgaagtgatcgagacaatcgagaacgacatcaagggcctgatcaacaccggcatcttcatcgagatcaagggccggttctaccagctgaaggaccacattctgcagttcgtgatccccaaccggggagtgggtaccaagcagctggtgaaaagcgagctggaagagaagaagtccgagctgcggcacaagctgaaatacgtgccccacgagtacatcgagctgatcgagatcgcccggaaccccacccaggacagaatcctggaaatgaaggtcatggaatttttcatgaaggtgtacggctaccggggcgagcacctgggcggcagcagaaaacccgacggcgccatctacaccgtgggcagccccatcgactacggcgtgatcgtggacaccaaggcctacagcggcggctacaacctgcccatcggacaggccgacgagatgcagagatacgtggaagagaaccagacccggaacaagcacatcaaccccaacgagtggtggaaggtgtaccccagcagcgtgaccgagttcaagttcctgttcgtgtccggccacttcaagggcaactacaaggcccagctgacccggctgaaccacatcaccaactgcaacggcgctgtgctgagcgtggaagaactgctgatcggcggcgagatgatcaaggccggcaccctgaccctggaagaagtgcggcggaagttcaacaacggcgagatcaacttc

SEQ ID NO: 12 - protein, 584 aa, Artificial Sequence
  Synthetic Construct
MFLSMVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVGTKQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 13 - DNA, 594 bp, Artificial Sequence
  modified FokI enzyme
atgaagcagctggtgaaaagcgagctggaagagaagaagtccgagctgcggcacaagctgaaatacgtgccccacgagtacatcgagctgatcgagatcgcccggaaccccacccaggacagaatcctggaaatgaaggtcatggaatttttcatgaaggtgtacggctaccggggcgagcacctgggcggcagcagaaaacccgacggcgccatctacaccgtgggcagccccatcgactacggcgtgatcgtggacaccaaggcctacagcggcggctacaacctgcccatcggacaggccgacgagatgcagagatacgtggaagagaaccagacccggaacaagcacatcaaccccaacgagtggtggaaggtgtaccccagcagcgtgaccgagttcaagttcctgttcgtgtccggccacttcaagggcaactacaaggcccagctgacccggctgaaccacatcaccaactgcaacggcgctgtgctgagcgtggaagaactgctgatcggcggcgagatgatcaaggccggcaccctgaccctggaagaagtgcggcggaagttcaacaacggcgagatcaacttc

SEQ ID NO: 14 - protein, 198 aa, Artificial Sequence
  Synthetic Construct
MKQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 15 - DNA, 615 bp, Artificial Sequence
  modified enzyme from SV40 and FokI
atgcccaagaagaagagaaaggtgaagcagctggtgaaaagcgagctggaagagaagaagtccgagctgcggcacaagctgaaatacgtgccccacgagtacatcgagctgatcgagatcgcccggaaccccacccaggacagaatcctggaaatgaaggtcatggaatttttcatgaaggtgtacggctaccggggcgagcacctgggcggcagcagaaaacccgacggcgccatctacaccgtgggcagccccatcgactacggcgtgatcgtggacaccaaggcctacagcggcggctacaacctgcccatcggacaggccgacgagatgcagagatacgtggaagagaaccagacccggaacaagcacatcaaccccaacgagtggtggaaggtgtaccccagcagcgtgaccgagttcaagttcctgttcgtgtccggccacttcaagggcaactacaaggcccagctgacccggctgaaccacatcaccaactgcaacggcgctgtgctgagcgtggaagaactgctgatcggcggcgagatgatcaaggccggcaccctgaccctggaagaagtgcggcggaagttcaacaacggcgagatcaacttc

SEQ ID NO: 16 - protein, 205 aa, Artificial Sequence
  Synthetic Construct
MPKKKRKVKQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 17 - DNA, 366 bp, Artificial Sequence
  modified ZFHD enzyme
atggagagaccctacgcctgccccgtggagagctgcgacagaagattcagcagaagcgacgagctgaccagacacatcagaatccacaccggccagaagcccttccagtgcagaatctgcatgagaaacttcagcagaagcgaccacctgaccacccacatcagaacccacacaggcggcggcagaagaagaaagaagagaaccagcatcgagaccaacatcagagtggccctggagaaaagcttcctggagaaccagaagcccaccagcgaggagatcaccatgatcgccgaccagctgaacatggagaaggaggtgatcagagtgtggttctgcaacagaagacagaaggagaagagaatcaac

SEQ ID NO: 18 - protein, 122 aa, Artificial Sequence
  Synthetic Construct
MERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRIN

SEQ ID NO: 19 - DNA, 387 bp, Artificial Sequence
  modified enzyme from SV40 and ZFHD
atgcccaagaagaagagaaaggtggagagaccctacgcctgccccgtggagagctgcgacagaagattcagcagaagcgacgagctgaccagacacatcagaatccacaccggccagaagcccttccagtgcagaatctgcatgagaaacttcagcagaagcgaccacctgaccacccacatcagaacccacacaggcggcggcagaagaagaaagaagagaaccagcatcgagaccaacatcagagtggccctggagaaaagcttcctggagaaccagaagcccaccagcgaggagatcaccatgatcgccgaccagctgaacatggagaaggaggtgatcagagtgtggttctgcaacagaagacagaaggagaagagaatcaac

SEQ ID NO: 20 - protein, 129 aa, Artificial Sequence
  Synthetic Construct
MPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRIN

SEQ ID NO: 21 - DNA, 963 bp, Artificial Sequence
  modified enzyme from ZFHD and FokI
atggagagaccctacgcctgccccgtggagagctgcgacagaagattcagcagaagcgacgagctgaccagacacatcagaatccacaccggccagaagcccttccagtgcagaatctgcatgagaaacttcagcagaagcgaccacctgaccacccacatcagaacccacacaggcggcggcagaagaagaaagaagagaaccagcatcgagaccaacatcagagtggccctggagaaaagcttcctggagaaccagaagcccaccagcgaggagatcaccatgatcgccgaccagctgaacatggagaaggaggtgatcagagtgtggttctgcaacagaagacagaaggagaagagaatcaacggtaccaagcagctggtgaaaagcgagctggaagagaagaagtccgagctgcggcacaagctgaaatacgtgccccacgagtacatcgagctgatcgagatcgcccggaaccccacccaggacagaatcctggaaatgaaggtcatggaatttttcatgaaggtgtacggctaccggggcgagcacctgggcggcagcagaaaacccgacggcgccatctacaccgtgggcagccccatcgactacggcgtgatcgtggacaccaaggcctacagcggcggctacaacctgcccatcggacaggccgacgagatgcagagatacgtggaagagaaccagacccggaacaagcacatcaaccccaacgagtggtggaaggtgtaccccagcagcgtgaccgagttcaagttcctgttcgtgtccggccacttcaagggcaactacaaggcccagctgacccggctgaaccacatcaccaactgcaacggcgctgtgctgagcgtggaagaactgctgatcggcggcgagatgatcaaggccggcaccctgaccctggaagaagtgcggcggaagttcaacaacggcgagatcaacttc

SEQ ID NO: 22 - protein, 321 aa, Artificial Sequence
  Synthetic Construct
MERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINGTKQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 23 - DNA, 984 bp, Artificial Sequence
  modified enzyme from SV40, ZFHD and FokI
atgcccaagaagaagagaaaggtggagagaccctacgcctgccccgtggagagctgcgacagaagattcagcagaagcgacgagctgaccagacacatcagaatccacaccggccagaagcccttccagtgcagaatctgcatgagaaacttcagcagaagcgaccacctgaccacccacatcagaacccacacaggcggcggcagaagaagaaagaagagaaccagcatcgagaccaacatcagagtggccctggagaaaagcttcctggagaaccagaagcccaccagcgaggagatcaccatgatcgccgaccagctgaacatggagaaggaggtgatcagagtgtggttctgcaacagaagacagaaggagaagagaatcaacggtaccaagcagctggtgaaaagcgagctggaagagaagaagtccgagctgcggcacaagctgaaatacgtgccccacgagtacatcgagctgatcgagatcgcccggaaccccacccaggacagaatcctggaaatgaaggtcatggaatttttcatgaaggtgtacggctaccggggcgagcacctgggcggcagcagaaaacccgacggcgccatctacaccgtgggcagccccatcgactacggcgtgatcgtggacaccaaggcctacagcggcggctacaacctgcccatcggacaggccgacgagatgcagagatacgtggaagagaaccagacccggaacaagcacatcaaccccaacgagtggtggaaggtgtaccccagcagcgtgaccgagttcaagttcctgttcgtgtccggccacttcaagggcaactacaaggcccagctgacccggctgaaccacatcaccaactgcaacggcgctgtgctgagcgtggaagaactgctgatcggcggcgagatgatcaaggccggcaccctgaccctggaagaagtgcggcggaagttcaacaacggcgagatcaacttc

SEQ ID NO: 24 - protein, 328 aa, Artificial Sequence
  Synthetic Construct
MPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINGTKQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 25 - DNA, 18 bp, Homo sapiens
tagggataacagggtaat

SEQ ID NO: 26 - DNA, 168 bp, adeno-associated virus 2
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacg

SEQ ID NO: 27 - DNA, 832 bp, Unknown
  cytomegalovirus
taggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaact

SEQ ID NO: 28 - DNA, 173 bp, Unknown
  cytomegalovirus
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgt

SEQ ID NO: 29 - DNA, 900 bp, Artificial Sequence
  modified construct from c-Myc NLS, FRAP and Homo sapiens
atggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 30 - protein, 300 aa, Artificial Sequence
  Synthetic Construct
MDYPAAKRVKLDSRILWHEMWHEGLEEASRLYFGERNVKGMFEVLEPLHAMMERGPQTLKETSFNQAYGRDLMEAQEWCRKYMKSGNVKDLLQAWDLYYHVFRRISKTRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 31 - DNA, 54 bp, Artificial Sequence
  modified construct from insect virus
gagggccgcggaagcttactaacatgcggtgacgtcgaggagaacccgggccct

SEQ ID NO: 32 - DNA, 145 bp, Artificial Sequence
  modified construct from zinc finger homeodomain and human IL-2 minimal promoter
aatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaac

SEQ ID NO: 33 - DNA, 1029 bp, Bacteriophage P1
  CDS (1)..(1029)
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgat

SEQ ID NO: 34 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 35 - DNA, 705 bp, Unknown
  yeast endonuclease
atgaagaacattaagaaaaatcaggtcatcaacctggggcccatttccaagctgctgaaagagtacaagtctcagctgatcgaactgaatattgagcagtttgaagcagggatcggcctgattctgggcgacgcctacatcaggagcagagatgagggaaagacctattgcatgcagttcgaatggaagaacaaagcttacatggaccacgtgtgcctgctgtatgatcagtgggtcctgtctccccctcacaagaaagagcgggtgaatcatctgggaaacctggtcattacttggggggcacagaccttcaagcatcaggccttcaacaagctggctaacctgtttattgtgaacaataagaagctgatccctaacaatctggtcgaaaactacctgacaccaatgagtctggcctattggttcatggacgatggcggaaaatgggactacaacaagaacagcctgaacaagagcatcgtgctgaacacccagtccttcacatttgaggaagtcgagtatctgctgaagggactgaggaacaagttccagctgaactgctacgtgaagattaacaagaacaagcccatcatctacatcgattctatgagttacctgatcttttataatctgattaagccatacctgatcccccagatgatgtataaactgcctaacacaatcagctccgagactttcctgaag

SEQ ID NO: 36 - protein, 235 aa, Unknown
  Synthetic Construct
MKNIKKNQVINLGPISKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKLIPNNLVENYLTPMSLAYWFMDDGGKWDYNKNSLNKSIVLNTQSFTFEEVEYLLKGLRNKFQLNCYVKINKNKPIIYIDSMSYLIFYNLIKPYLIPQMMYKLPNTISSETFLK

SEQ ID NO: 37 - DNA, 221 bp, Homo sapiens
cgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattcgggaaccaagctgg

SEQ ID NO: 38 - DNA, 491 bp, Unknown
  encephalomyocarditis virus
attttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatc

SEQ ID NO: 39 - DNA, 34 bp, Bacteriophage P1
ataacttcgtatagcatacattatacgaagttat

SEQ ID NO: 40 - DNA, 1650 bp, Unknown
  wild-type native firefly luciferase
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgtccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtg

SEQ ID NO: 41 - protein, 550 aa, Unknown
  Synthetic Construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELSAAVVVLEHGKTMTEKEIVDYVASQVTTAKKLRGGVVFVDEVPKGLTGKLDARKIREILIKAKKGGKIAV

SEQ ID NO: 42 - DNA, 239 bp, Simian virus 40
ggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatc

SEQ ID NO: 43 - DNA, 1650 bp, Artificial Sequence
  firefly luciferase
atggaggacgccaagaacatcaagaagggccccgcccccttctaccccctggaggacggcaccgccggagagcagctgcacaaggccatgaagagatacgccctggtgcccggcaccatcgccttcaccgacgcccacatcgaggtggacatcacctacgccgagtacttcgagatgagcgtgagactggccgaggccatgaagagatacggcctgaacaccaaccacagaatcgtggtgtgcagcgagaacagcctgcagttcttcatgcccgtgctgggagccctgttcatcggcgtggccgtggcccccgccaacgacatctacaacgagagagagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtataccttcgtgaccagccacctgccccccggcttcaacgagtacgacttcgtgcccgagagcttcgacagagacaagaccatcgccctgatcatgaacagcagcggcagcaccggcctgcccaagggcgtggccctgccccacagaaccgcctgcgtgagattcagccacgccagagaccccatcttcggcaaccagatcatccccgacaccgccatcctgagcgtggtgcccttccaccacggcttcggcatgttcaccaccctgggctacctgatctgcggcttcagagtggtgctgatgtatagattcgaggaggagctgttcctgagaagcctgcaggactacaagatccagagcgccctgctggtgcccaccctgttcagcttcttcgccaagagcaccctgatcgacaagtacgacctgagcaacctgcacgagatcgccagcggcggagcccccctgagcaaggaggtgggcgaggccgtggccaagagattccacctgcccggcatcagacagggctacggcctgaccgagaccaccagcgccatcctgatcacccccgagggcgacgacaagcccggagccgtgggcaaggtggtgcccttcttcgaggccaaggtggtggacctggacaccggcaagaccctgggcgtgaaccagagaggcgagctgtgcgtgagaggccccatgattatgtccggctacgtgaacaaccccgaggccaccaacgccctgatcgacaaggacggctggctgcacagcggcgacatcgcctactgggacgaggacgagcacttcttcatcgtggacagactgaagagcctgatcaagtacaagggctaccaggtggcccccgccgagctggagagcatcctgctgcagcaccccaacatcttcgacgccggagtggccggactgcccgacgacgacgccggagagctgcccgccgccgtggtggtgctggagcacggcaagaccatgaccgagaaggagatcgtggactacgtggccagccaggtgacaaccgccaagaagctgagaggcggcgtggtgttcgtggacgaggtgcccaagggcctgaccggcaagctggacgccagaaagatcagagagatcctgatcaaggccaagaagggcggcaagatcgccgtg

SEQ ID NO: 44 - protein, 550 aa, Artificial Sequence
  Synthetic Construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 45 - DNA, 411 bp, Artificial Sequence
  modified construct from c-Myc NLS, zinc finger homeodomain and FKBP
atggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactaga

SEQ ID NO: 46 - protein, 137 aa, Artificial Sequence
  Synthetic Construct
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTR

SEQ ID NO: 47 - DNA, 897 bp, Artificial Sequence
  modified construct from SV40 and FRAP
atgcccaagaagaagagaaaggtgatcctgtggcacgagatgtggcacgagggcctggaggaggccagcagactgtacttcggcgagagaaacgtgaagggcatgttcgaggtgctggagcccctgcacgccatgatggagagaggcccccagaccctgaaggagaccagcttcaaccaggcttacggcagagacctgatggaggcccaggagtggtgcagaaagtacatgaagtccggcaacgtgaaggacctgctgcaggcttgggacctgtactaccacgtgttcagaagaatcagcaagcagctgccccagctgactagtgacgagttccccaccatggtgttccccagcggccagatcagccaggccagcgccctggcccccgcccccccccaggtgctgccccaggcccccgcccccgcccccgcccccgccatggtgagcgccctggcccaggcccccgcccccgtgcccgtgctggcccccggccccccccaggccgtggccccccccgcccccaagcccacccaggccggagagggcaccctgagcgaggccctgctgcagctgcagttcgacgacgaggacctgggagccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcaccagactggtcacaggagcccagagaccccccgaccccgcccccgcccccctgggagcccccggcctgcccaacggcctgctcagcggcgacgaggacttcagcagcatcgccgacatggacttcagcgccctgctgagccagatcagcagc

SEQ ID NO: 48 - protein, 299 aa, Artificial Sequence
  Synthetic Construct
MPKKKRKVILWHEMWHEGLEEASRLYFGERNVKGMFEVLEPLHAMMERGPQTLKETSFNQAYGRDLMEAQEWCRKYMKSGNVKDLLQAWDLYYHVFRRISKQLPQLTSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 49 - DNA, 366 bp, Artificial Sequence; identical to SEQ ID NO: 17
  modified construct from ZFHD

SEQ ID NO: 50 - protein, 122 aa, Artificial Sequence; identical to SEQ ID NO: 18
  Synthetic Construct

SEQ ID NO: 51 - DNA, 387 bp, Artificial Sequence; identical to SEQ ID NO: 19
  modified construct from SV40 and ZFHD

SEQ ID NO: 52 - protein, 129 aa, Artificial Sequence; identical to SEQ ID NO: 20
  Synthetic Construct

SEQ ID NO: 53 - DNA, 324 bp, Unknown
  wild type FKBP
atgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 54 - protein, 108 aa, Unknown
  Synthetic Construct
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 55 - DNA, 321 bp, Artificial Sequence
  modified construct from FKBP
ggcgtgcaggtggagaccatcagccccggcgacggcagaaccttccccaagagaggccagacctgcgtggtgcactacaccggaatgctggaggacggcaagaagttcgacagcagcagagacagaaacaagcccttcaagttcatgctgggcaagcaggaggtgatcagaggctgggaggagggcgtggcccagatgagcgtgggccagagagccaagctgaccatcagccccgactacgcctacggagccaccggccaccccggcatcatccccccccacgccaccctggtgttcgacgtggagctgctgaagctggag

SEQ ID NO: 56 - DNA, 324 bp, Artificial Sequence
  modified construct from FKBP
ggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtga

SEQ ID NO: 57 - DNA, 321 bp, Artificial Sequence
  modified construct from FKBP
ggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagag

SEQ ID NO: 58 - DNA, 321 bp, Artificial Sequence
  modified construct from FKBP
ggcgtacaagtagagactataagtcctggtgatggaaggacttttccaaaaagaggacaaacatgtgtagttcattatacgggtatgttggaggacggcaaaaagttcgacagtagtagagatcgtaataaaccattcaaattcatgttgggtaaacaagaagtcattaggggatgggaggagggagtcgctcaaatgtcggttggacaacgtgctaagttaacaatcagccctgactacgcatacggagctacaggacatcctggtattatacctccccacgctaccttggtgtttgacgtcgaactgctgaagttagag